(19) United States
(12) Patent Application Publication
(10) Pub. No.: US 2024/0033366 A1
(43) Pub. Date: Feb. 1, 2024

(54) FUNCTIONALIZED SHIGA TOXIN B-SUBUNIT (STxB) PROTEINS AND CONJUGATES THEREOF

(71) Applicants: INSTITUT CURIE, Paris (FR); INSERM (INSTITUT NATIONAL DE LA SANTÉ ET DE LA RECHERCHE MÉDICALE), Paris (FR); CENTRE NATIONAL DE LA RECHERCHE SCIENTIFIQUE, Paris (FR); COMMISSARIAT À L’ÉNERGIE ATOMIQUE ET AUX ÉNERGIES ALTERNATIVES (CEA), Paris (FR); UNIVERSITE PARIS CITE, Paris (FR); APHP (ASSISTANCE PUBLIQUE - HÔPITAUX DE PARIS), Paris (FR)

(72) Inventors: Ludger JOHANNES, Courbevoie (FR); Anne BILLET, Houilles (FR); Jonathan ULMER, Bures-sur-Yvette (FR); Denis SERVENT, Versailles (FR); Gilles MOURIER, Villebon-sur-Yvette (FR); Pascal KESSLER, Orphin (FR); Eric TARTOUR, Paris (FR)

(73) Assignees: INSTITUT CURIE, Paris (FR); INSERM (INSTITUT NATIONAL DE LA SANTÉ ET DE LA RECHERCHE MÉDICALE), Paris (FR); CENTRE NATIONAL DE LA RECHERCHE SCIENTIFIQUE, Paris (FR); COMMISSARIAT À L’ÉNERGIE ATOMIQUE ET AUX ÉNERGIES ALTERNATIVES (CEA), Paris (FR); UNIVERSITE PARIS CITE, Paris (FR); APHP (ASSISTANCE PUBLIQUE - HÔPITAUX DE PARIS), Paris (FR)

(21) Appl. No.: 18/255,462

(22) PCT Filed: Dec. 2, 2021
(86) PCT No.: PCT/EP2021/084054

§ 371 Date: Jun. 1, 2023

(30) Foreign Application Priority Data
Dec. 2, 2020 (EP) 20306480.3

(51) Int. Cl. A61K 47/64; A61K 38/16

(52) U.S. Cl. CPC A61K 47/6415; A61K 38/164; A61K 2123/00

(57) ABSTRACT

Modified monomers of a Shiga toxin B-subunit (STxB) protein including at least one of: an addition of a reactive unnatural amino acid residue at the C-terminal extremity, and/or a substitution with a reactive unnatural amino acid residue at an amino acid position among Asp 3, Lys 8, Glu 10, Tyr 11, Lys 23, Lys 27, Thr 49, Lys 53, His 58, Asn 59, and Arg 69, reference made to the numbering of STxB from Shigella dysenteriae. Also relates to STxB conjugates, and oligomers, in particular pentamers, of these modified STxB proteins and STxB conjugates; as well as to compositions including the same and their use in treatment, vaccination and diagnosis methods.

### FIELD OF INVENTION

The present invention relates to modified monomers of a Shiga toxin B-subunit (STxB) protein comprising substitutions with, or additions of, reactive unnatural amino acid residues at one or several amino acid positions among Asp 3, Lys 8, Glu 10, Tyr 11, Lys 23, Lys 27, Thr 49, Lys 53, His 58, Asn 59, Arg 69 and the C-terminal extremity, reference made to the numbering of STxB from Shigella dysenteriae.

It also relates to STxB conjugates, and oligomers, in particular pentamers, of these modified STxB proteins and STxB conjugates; as well as to compositions comprising the same and their use in treatment, vaccination and diagnosis methods.

### BACKGROUND OF INVENTION

The capacity of antigen presenting cells, such as dendritic cells or macrophages, to process and present antigens on MHC class I and II molecules to T cells determines the successful stimulation of T cell adaptive immune responses. These adaptive immune responses are crucial to fight against infection or cancer cells.

Exogenous antigens internalized by endocytosis are processed within the endosomal system of antigen presenting cells into peptides that are loaded on MHC class II molecules, and transported to the cell surface where they can be recognized by antigen-specific CD4⁺ T cells.

Antigens which are present or have gained access to the host cell cytosol are processed mostly by proteasome into peptides and transported to the endoplasmic reticulum where they are loaded on MHC class I molecules in a process that has been termed cross-presentation. On the cell surface, MHC class I-peptide complexes are recognized by CD8⁺ cytotoxic T cells which play a crucial role in the elimination of viruses and intracellular bacteria as well as in the eradication of tumors.

However, many pathogens have evolved sophisticated strategies to elude antigen processing and the presentation machinery of antigen presenting cells, thereby ensuring survival within the host cells (Bhaysar et al., 2007. Nature. 449(7164):827-34). Similarly, tumor cells display characteristics which suppress their recognition and elimination from the organism (Whiteside, 2010. J Allergy Clin Immunol. 125(2 Suppl 2):S272-83).

There is thereof a need for vectors that are capable of delivering specific antigens into antigen presenting cells, as well as for immunological adjuvants which would boost inefficient host immune responses.

Several bacterial toxins have been intensively studied over the past decades in order to harness their abilities to enter host cells for the stimulation of adaptive T cell responses, direct elimination of cancer cells or to boost immunity as adjuvants.

Shiga toxin (STx) produced by Shigella dysenteriae and Shiga-like toxins produced by certain serotypes of Escherichia coli and some other bacteria (called STx1 or verotoxin 1; or STx2 or verotoxin 2) are responsible for serious medical conditions like dysentery, hemorrhagic colitis or hemolytic uremic syndrome (for a review, see Johannes & Römer, 2010. Nat Rev Microbiol. 8(2):105-16).

All these toxins belong to the AB family of protein toxins. The A-subunit (STxA) is a toxic moiety. After proteolytic activation by the host cell protease furin, it is then translocated into the cytosol of the host cell where it inhibits protein synthesis by modifying a conserved residue of 28S rRNA, thereby causing the cell death (Sandvig & van Deurs, 1996. Physiol Rev. 76(4):949-66; Tesh, 2010. Future Microbiol. 5(3):431-53). The B-subunit (STxB) is homopentameric and is responsible for STx binding to, and internalization into, target cells by interacting with globotriose-ceramide receptors (Gb3, also known as CD77) expressed on the surface of these cells (Sandvig & van Deurs, 1996. Physiol Rev. 76(4):949-66). The toxin is transported in a retrograde fashion from the plasma membrane via endosomes into Golgi apparatus and endoplasmic reticulum (Sandvig et al., 1992. Nature. 358(6386):510-2; Johannes et al., 1997. J Biol Chem. 272(31):19554-61).

Shiga toxins stimulate production of cytokines, such as IL-1, IL-6, IL-8, TNF-α or GM-CSF, in different cell types via activation of various mitogen-activated protein kinases (Thorpe et al., 1999. Infect Immun. 67(11):5985-93; Thorpe et al., 2001. Infect Immun. 69(10):6140-7; Smith et al., 2003. Infect Immun. 71(3):1497-504; Lee et al., 1998. Eur J Immunol. 28(9):2726-37).

Lee et al. (1998. Eur J Immunol. 28(9):2726-37) first reported that the non-toxic STxB subunit, carrying an epitope from a model tumor antigen (Mage 1), could be presented by human peripheral blood mononuclear cells in an MHC class-I restricted manner to Mage 1-specific cytotoxic T cells. No additional adjuvant was needed for the induction of specific anti-tumor cytotoxic T cells in mice after immunization with STxB carrying a mouse tumor epitope (Haicheur et al., 2000. J Immunol. 165(6):3301-8). It was later shown that vaccination with STxB carrying chemically coupled ovalbumin primed specific anti-OVA cytotoxic T cells and Tₕ1-polarized responses, and induced IgG2a antibodies (Haicheur et al., 2003. Int Immunol. 15(10):1161-71). Similarly, it was observed that the oral immunization with a fragment of the immunogenic rotavirus nonstructural protein 4 fragment linked to STxB induced protective humoral and cellular responses in mice (Choi et al., 2005. Vaccine. 23(44):5168-76).

Shiga toxin receptor Gb3 was shown to be expressed on malignant, even metastasizing cells (Kavbasnjuk et al., 2005. Proc Natl Acad Sci USA. 102(52):19087-92; Distler et al., 2009. PLoS One. 4(8):e6813). This can be exploited for the diagnosis of cancer as it was shown that STxB could reach Gb3-expressing digestive tumors in animal models as well as human colorectal tumors and their metastasis (Janssen et al., 2006. Cancer Res. 66(14):7230-6; Falguières et al., 2008. Mol Cancer Ther. 7(8):2498-508). A prodrug composition using topoisomerase I inhibitor SN38 coupled to STxB was also designed in order to specifically target cancer cells (El Alaoui et al., 2007. Angew Chem Int Ed Engl. 46(34):6469-72).

The binding of STxB to the Gb3 receptor make it a powerful tool for targeting various agents to Gb3-expressing cells. A major drawback is however the provision of STxB proteins conjugates.

Cysteine residues and their thiol functional groups have long been attractive targets for the selective modification of peptides and proteins (Means & Feeney, 1990. Bioconjug Chem. 1(1):2-12). From a bioconjugation standpoint, the most enticing trait of cysteines is their ability to undergo highly selective ligations via Michael additions and alkylations (Patterson et al., 2014. Bioconjug Chem. 25(8):1402-7; Toda et al., 2013. Angew Chem Int Ed Engl. 52(48):12592-6; Badescu et al., 2014. Bioconjug Chem. 25(3):460-9).

However, this strategy is accompanied by the loss of intramolecular disulfide bonds. In the case of STxB, only two cysteines are present in the amino acid sequence, forming a disulfide bond, which is crucial for protein folding and stability.

An alternative to the use of native cysteine residues lies in the genetic incorporation of engineered cysteines as bespoke conjugation sites. However, this approach shows both advantages and drawbacks. Although it allows to keep intact the native cysteine residues forming disulfide bonds, it has been shown that free, unpaired cysteine residues can spontaneously oxidize to form undesired disulfide bridges, leading to aggregation and structural modifications (Woo et al., 1991. J Biol Chem. 266(28):18419-22; Wootton & Yoo, 2003. J Virol. 77(8):4546-57). Moreover, the location of the incorporation site must be chosen very carefully in order to eliminate the risk of interference with, e.g., the Gb3 binding domain.

Tobola et al. (2019. Interface Focus. 9(2):20180072) has described a method to produce, in E. coli, a recombinant STxB protein, modified with an azidolysine (Azk) in lieu of a lysine residue at position 8, using the stop-codon suppression (SCS) technique. However, the production of this modified recombinant STxB protein was highly contaminated with unmodified (wild-type) STxB protein and yielded a composition comprising only around 37% of modified monomer of STxB.

There remains therefore a need for the provision of homogeneous compositions of STxB proteins which can be readily conjugated, while remaining conformationally stable.

Here, the Inventors provide a solution to this remaining need.

### SUMMARY

The present invention relates to a composition comprising at least 50% of isolated, modified monomer of a Shiga toxin B-subunit (STxB) protein or of a variant thereof, wherein said monomer of the STxB protein or of the variant thereof comprises a least one of:

an addition of a reactive unnatural amino acid residue at the C-terminal extremity, and/or
a substitution with a reactive unnatural amino acid residue at one or several amino acid positions selected from the group consisting of Asp 3, Lys 8, Glu 10, Tyr 11, Lys 23, Lys 27, Thr 49, Lys 53, His 58, Asn 59, and Arg 69, reference made to SEQ ID NO: 2 numbering, or at an equivalent position in a variant thereof.

In one embodiment, the monomer of the STxB protein or of the variant thereof does not comprise a substitution with, or an addition of, a reactive unnatural amino acid residue at amino acid positions Thr 1, Thr 6 and Asp 26, according to SEQ ID NO: 2 numbering, or at equivalent positions in a variant thereof.

In one embodiment, the reactive unnatural amino acid residue comprises a functional group selected from the group consisting of azide, alkyne, aldehyde, keto, beta-diketo, alkoxyamine, acyl hydrazide, dehydroalanine, thioester, ester, boronate, halide, acetylenic, olefinic, vicinal thiol amine, and norbornene moieties.

In one embodiment, the reactive unnatural amino acid residue is selected from the group consisting of 6-azido-L-lysine, 3-azido-L-alanine and 4-azidomethyl-L-phenylalanine.

In one embodiment, the monomer of the STxB protein or of the variant thereof is selected from the group consisting of:
a STxB protein comprising an addition in C-terminal of a 3-azido-L-alanine,
a STxB protein comprising an addition in C-terminal of a 6-azido-L-lysine,
a STxB protein comprising a substitution of Asp 3 into 6-azido-L-lysine,
a STxB protein comprising a substitution of Lys 8 into 6-azido-L-lysine,
a STxB protein comprising a substitution of Glu 10 into 6-azido-L-lysine,
a STxB protein comprising a substitution of Tyr 11 into 6-azido-L-lysine,
a STxB protein comprising a substitution of Tyr 11 into 4-azidomethyl-L-phenylalanine,
a STxB protein comprising a substitution of Lys 23 into 6-azido-L-lysine,
a STxB protein comprising a substitution of Lys 27 into 6-azido-L-lysine,
a STxB protein comprising a substitution of Thr 49 into 6-azido-L-lysine,
a STxB protein comprising a substitution of Lys 53 into 6-azido-L-lysine,
a STxB protein comprising a substitution of His 58 into 3-azido-L-alanine,
a STxB protein comprising a substitution of Asn 59 into 6-azido-L-lysine, and
a STxB protein comprising a substitution of Arg 69 into 6-azido-L-lysine; reference made to SEQ ID NO: 2 numbering, or at an equivalent position in a variant thereof.

In one embodiment, the monomer of the STxB protein or of the variant thereof has an amino acid sequence with at least 75% global sequence identity to an amino acid sequence selected from the group comprising SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 and SEQ ID NO: 20; preferably to the amino acid sequence of SEQ ID NO: 2.

In one embodiment, the monomer of the STxB protein or of the variant thereof further comprises a substitution of Met 48 with L-norleucine, reference made to SEQ ID NO: 2 numbering, or at an equivalent position in a variant thereof.

In one embodiment, the said monomer of the STxB protein or of the variant thereof is not a recombinant protein.

In one embodiment, the modified monomer of the STxB protein or of the variant thereof is a conjugate, comprising a payload bound thereto through the reactive unnatural amino acid residue, optionally through via a linker.

The present invention also relates to a composition comprising at least 50% of STxB monomer conjugate, comprising a monomer of a STxB protein or of a variant thereof, bound to a payload, optionally through a linker, at an amino acid position selected from the group consisting of the C-terminal extremity, Asp 3, Lys 8, Glu 10, Tyr 11, Lys 23, Lys 27, Thr 49, Lys 53, His 58, Asn 59, and Arg 69, reference made to SEQ ID NO: 2 numbering, or at an equivalent position in a variant thereof.

In one embodiment, the payload is selected from the group consisting of chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, antigens, hormones, coding or non-coding oligonucleotides, photodetectable labels, contrast agents, and radiolabels.

The present invention also relates to a composition comprising at least 50% of modified pentamers of a Shiga toxin B-subunit (STxB) protein or of a variant thereof, said modified pentamers of the STxB protein or of the variant thereof comprising at least one modified monomer according to the present invention; preferably said modified pentamers of the STxB protein or of the variant thereof comprise five modified monomers according to the present invention.

In one embodiment, the modified pentamers of the STxB protein or of the variant thereof are conjugates comprising at least one STxB monomer conjugate according to the invention.

The present invention also relates to a composition comprising at least 50% of Shiga toxin B-subunit (STxB) pentamer conjugates, said STxB pentamer conjugates comprising at least one STxB monomer conjugate according to the present invention.

In one embodiment, the modified pentamers of the STxB protein or of the variant thereof, and the STxB pentamer conjugates, retain their ability to bind to the glycosphingolipid Gb3/CD77.

The present invention also relates to the compositions according to the present invention, for use in treating a disease in a subject in need thereof, optionally wherein the disease is selected from cancer, infectious diseases, immune disorders and inflammatory disorders; or for use in vaccinating in a subject in need thereof;

preferably wherein the STxB monomer or oligomer conjugate comprises a payload selected from the group consisting of chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, antigens, hormones, and coding or non-coding oligonucleotides.

The present invention also relates to the compositions according to the present invention, for use as a contrast agent in a method of medical imaging of a subject in need thereof; or for use in an in vivo method of diagnosing a disease in a subject in need thereof, optionally wherein the disease is selected from cancer, infectious diseases, immune disorders and inflammatory disorders;

preferably wherein the STxB monomer or oligomer conjugate comprises a payload selected from the group consisting of photodetectable labels, contrast agents and radiolabels.

### DETAILED DESCRIPTION

The present invention relates to a modified monomer of a Shiga toxin B-subunit (STxB) protein or of a variant thereof.

In one embodiment, the STxB protein is STxB from Shigella dysenteriae, with Uniprot accession number Q7BQ98-1 (SEQ ID NO: 1).

In one embodiment, the STxB protein is STxB from Shigella dysenteriae, with Uniprot accession number Q7BQ98-1, devoid of its signal peptide. The signal peptide of STxB from Shigella dysenteriae corresponds to amino acid residues 1 to 20 of Uniprot accession number Q7BQ98-1 (SEQ ID NO: 1).

In one embodiment, the STxB protein is STxB from Shigella dysenteriae devoid of its signal peptide (SEQ ID NO: 2).

SEQ ID NO: 1
MKKTLLIAASLSFFSASALATPDCVTGKVEYTKYNDDDTFTVKVGDKEL
FTNRWNLQSLLLSAQITGMTVTIKTNACHNGGGFSEVIFR
SEQ ID NO: 2
TPDCVTGKVEYTKYNDDDTFTVKVGDKELFTNRWNLQSLLLSAQITGMT
VTIKTNACHNGGGFSEVIFR

Unless mentioned otherwise, the term “STxB” is used herein to refer to a STxB protein or a variant thereof, which is devoid of its signal peptide.

In one embodiment, the STxB protein or the variant thereof has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 2.

In one embodiment, the STxB protein or the variant thereof has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% global sequence identity or more to the amino acid sequence set forth in SEQ ID NO: 2.

As used herein, the term “sequence identity” refers to the number of identical or similar amino acids in a comparison between a test and a reference polypeptide. Sequence identity can be determined by sequence alignment of protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps, as follows:

$$sequence identity = \frac{number of identical positions}{lengths of the total aligned sequence} \times 100$$

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences, each of 100 nucleotides in length, 50% of the residues are the same. It is understood that global alignment can also be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman & Wunsch, 1970. J Mol Biol. 48(3):443-53). Exemplary programs and software for performing global alignment are publicly available and include the Global Sequence Alignment Tool available at the National Center for Biotechnology Information (NCBI) website (http://ncbi.nlm.nih.gov), and the program available at http://deepc2.psi.iastate.edu/aat/align/align.html. A “global alignment” determines a “global sequence identity”.

As used herein, a “local alignment” is an alignment that aligns two sequence, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4):482-9). For example, 50% sequence identity based on “local alignment” means that in an alignment of the full sequence of two compared sequences of any length, a region of similarity or identity of 100 nucleotides in length has 50% of the residues that are the same in the region of similarity or identity. A “local alignment” determines a “local sequence identity”.

For purposes herein, sequence identity can be determined by standard alignment algorithm programs used with default gap penalties established by each supplier. Default parameters for the GAP program can include:
(1) a unary comparison matrix (containing a value of 1 for identities and 0 for non-identities) and the weighted comparison matrix of Gribskov & Burgess (1986. Nucleic Acids Res. 14(10:6745-63), as described by Schwartz & Dayhoff (1979. Matrices for detecting distant relationships. In Dayhoff (Ed.), Atlas of protein sequences. 5:353-358. Washington, DC: National Biomedical Research Foundation);
(2) a penalty of 3.0 for each gap and an additional 0.10 penalty for each symbol in each gap; and
(3) no penalty for end gaps.

Whether any two polypeptides have amino acid sequences that are at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, %, 91%, 92%, 93%, 94%, 95%, 96%, 97%, 98%, 99% or more “identical”, or other similar variations reciting a percent identity, can be determined using known computer algorithms based on local or global alignment (see, e.g., wikipedia.org/wiki/Sequence_alignment_software, providing links to dozens of known and publicly available alignment databases and programs).

Generally, for purposes herein, sequence identity is determined using computer algorithms based on global alignment, such as the Needleman-Wunsch Global Sequence Alignment tool available from NCBI/BLAST (http://blast.ncbi.nlm.nih.gov/Blast.cgi?Web&Page_BlastHome); LAlign (William Pearson implementing the Huang and Miller algorithm [Huang & Miller, 1991. Adv Appl Math. 12(3):337-57); and program from Xiaoqui Huang available at http://deepc2.psi.iastate.edu/aat/align/align.html.

Typically, the full-length sequence of each of the compared polypeptides is aligned across the full-length of each sequence in a global alignment. Local alignment also can be used when the sequences being compared are substantially the same length.

Therefore, as used herein, the term “identity” represents a comparison or alignment between a test and a reference polypeptide.

In one exemplary embodiment, “at least 60% of sequence identity” refers to percent identities from 60 to 100% relative to the reference polypeptide. Identity at a level of 60% or more is indicative of the fact that, assuming for exemplification purposes a test and reference polypeptide length of 100 amino acids are compared, no more than % (i.e., 40 out of 100) of amino acids in the test polypeptide differ from those of the reference polypeptide. Such differences can be represented as point mutations randomly distributed over the entire length of an amino acid sequence or they can be clustered in one or more locations of varying length up to the maximum allowable, e.g., 40/100 amino acid difference (approximately 60% identity). Differences can also be due to deletions or truncations of amino acid residues. Differences are defined as amino acid substitutions, insertions or deletions. Depending on the length of the compared sequences, at the level of homologies or identities above about 85-90%, the result can be independent of the program and gap parameters set; such high levels of identity can be assessed readily, often without relying on software.

According to the invention, the modified monomer of the STxB protein or of the variant thereof comprises a substitution with, or an addition of, a reactive unnatural amino acid residue at one or several, such as 1, 2, 3, 4, 5, 6, 7, 8, 9, 10, 11 or 12, amino acid positions selected from the group comprising or consisting of the C-terminal extremity (i.e., after Arg 69), Asp 3, Lys 8, Glu 10, Tyr 11, Lys 23, Lys 27, Thr 49, Lys 53, His 58, Asn 59, and Arg 69, reference made to SEQ ID NO: 2 numbering, or at one or several equivalent positions in a variant thereof.

By “unnatural amino acid residue”, it is meant an amino acid residue that is not are not found in natural polypeptide chains; in other words, any amino acid residue other than any of the 22 proteinogenic amino acid residues (i.e., L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, L-valine, L-selenocysteine and L-pyrrolysine).

In one embodiment, the modified monomer of the STxB protein or of the variant thereof comprises a substitution with, or an addition of, a reactive unnatural amino acid residue at one or several, such as 1, 2, 3, 4, 5, 6, 7, 8, 9, 10 or 11, amino acid positions selected from the group comprising or consisting of the C-terminal extremity (i.e., after Arg 69), Asp 3, Glu 10, Tyr 11, Lys 23, Lys 27, Thr 49, Lys 53, His 58, Asn 59, and Arg 69, reference made to SEQ ID NO: 2 numbering, or at one or several equivalent positions in a variant thereof.

In one embodiment, the modified monomer of the STxB protein or of the variant thereof comprises one addition of a reactive unnatural amino acid residue at the C-terminal extremity (i.e., after Arg 69), reference made to SEQ ID NO: 2 numbering, or at one equivalent position in a variant thereof.

In one embodiment, the modified monomer of the STxB protein or of the variant thereof comprises a substitution with a reactive unnatural amino acid residue at one or several, such as 1, 2, 3, 4, 5, 6, 7, 8, 9, 10 or 11, amino acid positions selected from the group comprising or consisting of Asp 3, Lys 8, Glu 10, Tyr 11, Lys 23, Lys 27, Thr 49, Lys 53, His 58, Asn 59 and Arg 69, reference made to SEQ ID NO: 2 numbering, or at one or several equivalent positions in a variant thereof.

In one embodiment, the modified monomer of the STxB protein or of the variant thereof comprises a substitution with a reactive unnatural amino acid residue at one or several, such as 1, 2, 3, 4, 5, 6, 7, 8, 9, 10 or 11, amino acid positions selected from the group comprising or consisting of Asp 3, Glu 10, Tyr 11, Lys 23, Lys 27, Thr 49, Lys 53, His 58, Asn 59 and Arg 69, reference made to SEQ ID NO: 2 numbering, or at one or several equivalent positions in a variant thereof.

In one embodiment, the modified monomer of the STxB protein or of the variant thereof comprises a substitution with, or an addition of, a reactive unnatural amino acid residue at one amino acid position selected from the group comprising or consisting of Asp 3, Lys 8, Glu 10, Tyr 11, Lys 23, Lys 27, Thr 49, Lys 53, His 58, Asn 59, Arg 69 and the C-terminal extremity (i.e., after Arg 69), reference made to SEQ ID NO: 2 numbering, or at one equivalent position in a variant thereof.

In one embodiment, the modified monomer of the STxB protein or of the variant thereof comprises a substitution with, or an addition of, a reactive unnatural amino acid residue at one amino acid position selected from the group comprising or consisting of Asp 3, Glu 10, Tyr 11, Lys 23, Lys 27, Thr 49, Lys 53, His 58, Asn 59, Arg 69 and the C-terminal extremity (i.e., after Arg 69), reference made to SEQ ID NO: 2 numbering, or at one equivalent position in a variant thereof.

In one embodiment, the modified monomer of the STxB protein or of the variant thereof comprises a substitution with a reactive unnatural amino acid residue at one amino acid position selected from the group comprising or consisting of Asp 3, Lys 8, Glu 10, Tyr 11, Lys 23, Lys 27, Thr 49, Lys 53, His 58, Asn 59 and Arg 69, reference made to SEQ ID NO: 2 numbering, or at one equivalent position in a variant thereof.

In one embodiment, the modified monomer of the STxB protein or of the variant thereof comprises a substitution with a reactive unnatural amino acid residue at one amino acid position selected from the group comprising or consisting of Asp 3, Glu 10, Tyr 11, Lys 23, Lys 27, Thr 49, Lys 53, His 58, Asn 59 and Arg 69, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Asp 3, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Lys 8, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Glu 10, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Tyr 11, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Lys 23, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Lys 27, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Thr 49, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Lys 53, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position His 58, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Asn 59, reference made to SEQ ID NO: 2 numbering, or at one equivalent position in a variant thereof.

In one embodiment, the modified monomer of the STxB protein or of the variant thereof comprises one substitution with a reactive unnatural amino acid residue at amino acid position Arg 69, reference made to SEQ ID NO: 2 numbering, or at one equivalent position in a variant thereof.

In one embodiment, the modified monomer of the STxB protein or of the variant thereof does not comprise a substitution with a reactive unnatural amino acid residue at one or several amino acid positions selected from the group comprising or consisting of Thr 1, Thr 6 and Asp 26, according to SEQ ID NO: 2 numbering, or at one or several equivalent positions in a variant thereof.

In one embodiment, the modified monomer of the STxB protein or of the variant thereof does not comprise one substitution with a reactive unnatural amino acid residue at amino acid position Thr 1, according to SEQ ID NO: 2 numbering, or at one equivalent position in a variant thereof.

In one embodiment, the modified monomer of the STxB protein or of the variant thereof does not comprise one substitution with a reactive unnatural amino acid residue at amino acid position Thr 6, according to SEQ ID NO: 2 numbering, or at one equivalent position in a variant thereof.

In one embodiment, the modified monomer of the STxB protein or of the variant thereof does not comprise one substitution with a reactive unnatural amino acid residue at amino acid position Asp 26, according to SEQ ID NO: 2 numbering, or at one equivalent position in a variant thereof.

In one embodiment, the modified monomer of the STxB protein or of the variant thereof does not comprise a substitution with a reactive unnatural amino acid residue at amino acid positions Thr 1, Thr 6 and Asp 26, according to SEQ ID NO: 2 numbering, or at equivalent positions in a variant thereof.

By “reactive unnatural amino acid residue”, it is meant an amino acid residue bearing a functional group, i.e., a group that can be reacted in suitable conditions with a chemically reactive moiety of, e.g., another molecule, to form a conjugate. Examples of such functional groups include, but are not limited to, azide, alkyne, aldehyde, keto, beta-diketo, alkoxyamine, acyl hydrazide, dehydroalanine, thioester, ester, boronate, halide, acetylenic, olefinic, vicinal thiol amine, and norbornene moieties.

In one embodiment, the reactive unnatural amino acid residue is selected from azide-functionalized amino acid residues. Such azide-functionalized amino acid residues include, but are not limited to, 3-azido-D-alanine, 3-azido-L-alanine, 4-azido-D-homoalanine, 4-azido-L-phenylalanine, 4-azidomethyl-D-phenylalanine, 4-azido-L-homoalanine, 4-azido-D-phenylalanine, 4-azidomethyl-L-phenylalanine, 5-azido-D-ornithine, 5-azido-L-ornithine, 6-azido-D-lysine, and 6-azido-L-lysine.

In one embodiment, the reactive unnatural amino acid residue is selected from the group comprising or consisting of 6-azido-L-lysine, 3-azido-L-alanine and 4-azidomethyl-L-phenylalanine.

In one embodiment, the reactive unnatural amino acid residue is not N⁶-[(2-azidoethoxy)carbonyl]-L-lysine.

In one embodiment, the modified monomer of the STxB protein or of the variant thereof is selected from the group comprising or consisting of:
a STxB protein comprising an addition at the C-terminal extremity of a 3-azido-L-alanine,
a STxB protein comprising an addition at the C-terminal extremity of a 6-azido-L-lysine,
a STxB protein comprising a substitution of Asp 3 into 6-azido-L-lysine,
a STxB protein comprising a substitution of Lys 8 into 6-azido-L-lysine,
a STxB protein comprising a substitution of Glu 10 into 6-azido-L-lysine,
a STxB protein comprising a substitution of Tyr 11 into 6-azido-L-lysine,
a STxB protein comprising a substitution of Tyr 11 into 4-azidomethyl-L-phenylalanine,
a STxB protein comprising a substitution of Lys 23 into 6-azido-L-lysine,
a STxB protein comprising a substitution of Lys 27 into 6-azido-L-lysine,
a STxB protein comprising a substitution of Thr 49 into 6-azido-L-lysine,
a STxB protein comprising a substitution of Lys 53 into 6-azido-L-lysine,
a STxB protein comprising a substitution of His 58 into 3-azido-L-alanine,
a STxB protein comprising a substitution of Asn 59 into 6-azido-L-lysine, and
a STxB protein comprising a substitution of Arg 69 into 6-azido-L-lysine;
   reference made to SEQ ID NO: 2 numbering, or at an equivalent position in a variant thereof.

In one embodiment, the modified monomer of the STxB protein or of the variant thereof comprises a substitution at amino acid position Met 48 with L-norleucine, reference made to SEQ ID NO: 2 numbering, or at an equivalent position in a variant thereof.

In one embodiment, the modified monomer of the STxB protein or of the variant thereof comprises a substitution with, or an addition of, a reactive unnatural amino acid residue as described above; and further comprises a substitution at amino acid position Met 48 with L-norleucine, reference made to SEQ ID NO: 2 numbering, or at an equivalent position in a variant thereof.

Also encompassed in the present invention modified monomers of variants of the STxB protein. As used herein, the term “variant” is meant to encompass homologs, fragments and mutants of the STxB protein, including combinations thereof.

As used herein, the term “homolog” with reference to the STxB protein from Shigella dysenteriae described above, refers to a distinct protein from another family or species which is determined by functional, structural or genomic analyses to correspond to the original STxB protein from Shigella dysenteriae. Most often, homologs will have functional, structural, or genomic similarities. Techniques are known by which homologs of a protein can readily be cloned using genetic probes and PCR. The identity of cloned sequences as homologous can be confirmed using functional assays and/or by genomic mapping of the genes.

In one embodiment, a homolog of the STxB protein from Shigella dysenteriae described above is a STxB protein from bacteria of the genus Escherichia. These Escherichia bacteria are commonly named “Shiga toxin-producing Escherichia coli” or “STEC”, although STxB proteins have been identified in at least one other species of the genus Escherichia (Brandal et al., 2015. J Clin Microbiol. 53(4):1454-5).

STxB proteins from Escherichia may be classified in two distinct types: STx1B and STx2B, and further divided into several subtypes: STx1B, STx1cB, STx1 dB, STx2B, STx2cB, STx2 dB, STx2eB, STx2fB and STx2gB.

In one embodiment, a homolog of the STxB protein from Shigella dysenteriae is a STxB protein from bacteria of the genus Escherichia, selected from the group comprising or consisting of STx1B, STx1cB, STx1 dB, STx2B, STx2cB, STx2 dB, STx2eB, STx2fB and STx2gB.

In one embodiment, a homolog of the STxB protein from Shigella dysenteriae described above is STx1B from Escherichia coli, with Uniprot accession number Q8X4M7-1 (SEQ ID NO: 1). In one embodiment, a homolog of the STxB protein from Shigella dysenteriae described above is STx1B from Escherichia coli, with Uniprot accession number Q8X4M7-1, devoid of its signal peptide. The signal peptide of STx1B from Escherichia coli corresponds to amino acid residues 1 to 20 of Uniprot accession number Q8X4M7-1 (SEQ ID NO: 1). In one embodiment, a homolog of the STxB protein from Shigella dysenteriae described above is STx1B from Escherichia coli devoid of its signal peptide (SEQ ID NO: 2). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, %, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 2.

In one embodiment, a homolog of the STxB protein from Shigella dysenteriae described above is STx1cB from Escherichia coli, with Uniprot accession number Q47641-1 (SEQ ID NO: 3). In one embodiment, a homolog of the STxB protein from Shigella dysenteriae described above is STx1cB from Escherichia coli, with Uniprot accession number Q47641-1, devoid of its signal peptide. The signal peptide of STx1cB from Escherichia coli corresponds to amino acid residues 1 to 20 of Uniprot accession number Q47641-1 (SEQ ID NO: 3). In one embodiment, a homolog of the STxB protein from Shigella dysenteriae described above is STx1cB from Escherichia coli devoid of its signal peptide (SEQ ID NO: 4). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 4.

SEQ ID NO: 3
MKKILLIAASLSFFSASVLAAPDCVTGKVEYTKYNDDDTFTVKVGDKEL
FTNRWNLQSLLLSAQITGMTVTIKTNACHNGGGFSEVIFR
SEQ ID NO: 4
APDCVTGKVEYTKYNDDDTFTVKVGDKELFTNRWNLQSLLLSAQITGMT
VTIKTNACHNGGGFSEVIFR

In one embodiment, a homolog of the STxB protein from Shigella dysenteriae described above is STx1 dB from Escherichia coli, with Uniprot accession number Q83XK2-1 (SEQ ID NO: 5). In one embodiment, a homolog of the STxB protein from Shigella dysenteriae described above is STx1 dB from Escherichia coli, with Uniprot accession number Q83XK2-1, devoid of its signal peptide. The signal peptide of STx1 dB from Escherichia coli corresponds to amino acid residues 1 to 20 of Uniprot accession number Q83XK2-1 (SEQ ID NO: 5). In one embodiment, a homolog of the STxB protein from Shigella dysenteriae described above is STx1 dB from Escherichia coli devoid of its signal peptide (SEQ ID NO: 6). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 6.

SEQ ID NO: 5
MKKVLLIAVSLSFLSASVLAAPDCVTGKVEYTKYNDDDTFTVKVADKEL
FTNRWNLQSLLLSAQITGMTVTIKTTACHNGGGFSEVIFR
SEQ ID NO: 6
APDCVTGKVEYTKYNDDDTFTVKVADKELFTNRWNLQSLLLSAQITGMT
VTIKTTACHNGGGFSEVIFR

In one embodiment, a homolog of the STxB protein from Shigella dysenteriae described above is STx2B from Escherichia coli, with Uniprot accession number Q8X531-1 (SEQ ID NO: 7). In one embodiment, a homolog of the STxB protein from Shigella dysenteriae described above is STx2B from Escherichia coli, with Uniprot accession number Q8X531-1, devoid of its signal peptide. The signal peptide of STx2B from Escherichia coli corresponds to amino acid residues 1 to 19 of Uniprot accession number Q8X531-1 (SEQ ID NO: 7). In one embodiment, a homolog of the STxB protein from Shigella dysenteriae described above is STx2B from Escherichia coli devoid of its signal peptide (SEQ ID NO: 8). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 8.

SEQ ID NO: 7
MKKMFMAVLFALASVNAMAADCAKGKIEFSKYNEDDTFTVKVDGKEYWT
SRWNLQPLLQSAQLTGMTVTIKSSTCESGSGFAEVQFNND
SEQ ID NO: 8
ADCAKGKIEFSKYNEDDTFTVKVDGKEYWTSRWNLQPLLQSAQLTGMTV
TIKSSTCESGSGFAEVQFNND

In one embodiment, a homolog of the STxB protein from Shigella dysenteriae described above is STx2cB from Escherichia coli, with Uniprot accession number Q07871-1 (SEQ ID NO: 9). In one embodiment, a homolog of the STxB protein from Shigella dysenteriae described above is STx2cB from Escherichia coli, with Uniprot accession number Q07871-1, devoid of its signal peptide. The signal peptide of STx2cB from Escherichia coli corresponds to amino acid residues 1 to 19 of Uniprot accession number Q07871-1 (SEQ ID NO: 9). In one embodiment, a homolog of the STxB protein from Shigella dysenteriae described above is STx2cB from Escherichia coli devoid of its signal peptide (SEQ ID NO: 10). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 10.

SEQ ID NO: 9
MKKMFMAVLFALVSVNAMAADCAKGKIEFSKYNENDTFTVKVAGKEYWT
SRWNLQPLLQSAQLTGMTVTIKSSTCESGSGFAEVQFNND
SEQ ID NO: 10
ADCAKGKIEFSKYNENDTFTVKVAGKEYWTSRWNLQPLLQSAQLTGMTV
TIKSSTCESGSGFAEVQFNND

In one embodiment, a homolog of the STxB protein from Shigella dysenteriae described above is STx2 dB from Escherichia coli, with Uniprot accession number Q8GGL0-1 (SEQ ID NO: 11). In one embodiment, a homolog of the STxB protein from Shigella dysenteriae described above is STx2 dB from Escherichia coli, with Uniprot accession number Q8GGL0-1, devoid of its signal peptide. The signal peptide of STx2 dB from Escherichia coli corresponds to amino acid residues 1 to 19 of Uniprot accession number Q8GGL0-1 (SEQ ID NO: 11). In one embodiment, a homolog of the STxB protein from Shigella dysenteriae described above is STx2 dB from Escherichia coli devoid of its signal peptide (SEQ ID NO: 12). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 12.

SEQ ID NO: 11
MKKMFMAVLFALVSVNAMAADCAKGKIEFSKYNENDTFTVKVDGKEYWT
SRWNLQPLLQSAQLTGMTVTIKSSTCASGSGFAEVQFNND
SEQ ID NO: 12
ADCAKGKIEFSKYNENDTFTVKVDGKEYWTSRWNLQPLLQSAQLTGMTV
TIKSSTCASGSGFAEVQFNND

In one embodiment, a homolog of the STxB protein from Shigella dysenteriae described above is STx2eB from Escherichia coli, with Uniprot accession number Q47644-1 (SEQ ID NO: 13). In one embodiment, a homolog of the STxB protein from Shigella dysenteriae described above is STx2eB from Escherichia coli, with Uniprot accession number Q47644-1, devoid of its signal peptide. The signal peptide of STx2eB from Escherichia coli corresponds to amino acid residues 1 to 19 of Uniprot accession number Q47644-1 (SEQ ID NO: 13). In one embodiment, a homolog of the STxB protein from Shigella dysenteriae described above is STx2eB from Escherichia coli devoid of its signal peptide (SEQ ID NO: 14). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 14.

SEQ ID NO: 13
MKKMFIAVLFALVSVNAMAADCAKGKIEFSKYNEDNTFTVKVSGREYWT
NRWNLQPLLQSAQLTGMTVTIISNTCSSGSGFAQVKFN
SEQ ID NO: 14
ADCAKGKIEFSKYNEDNTFTVKVSGREYWTNRWNLQPLLQSAQLTGMTV
TIISNTCSSGSGFAQVKFN

In one embodiment, a homolog of the STxB protein from Shigella dysenteriae described above is STx2fB from Escherichia coli, with Uniprot accession number Q47646-1 (SEQ ID NO: 15). In one embodiment, a homolog of the STxB protein from Shigella dysenteriae described above is STx2fB from Escherichia coli, with Uniprot accession number Q47646-1, devoid of its signal peptide. The signal peptide of STx2fB from Escherichia coli corresponds to amino acid residues 1 to 19 of Uniprot accession number Q47646-1 (SEQ ID NO: 15). In one embodiment, a homolog of the STxB protein from Shigella dysenteriae described above is STx2fB from Escherichia coli devoid of its signal peptide (SEQ ID NO: 16). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 16.

SEQ ID NO: 15
MKKMIIAVLFGLFSANSMAADCAVGKIEFSKYNEDDTFTVKVSGREYWT
NRWNLQPLLQSAQLTGMTVTIISNTCSSGSGFAQVKFN
SEQ ID NO: 16
ADCAVGKIEFSKYNEDDTFTVKVSGREYWTNRWNLQPLLQSAQLTGMTV
TIISNTCSSGSGFAQVKFN

In one embodiment, a homolog of the STxB protein from Shigella dysenteriae described above is STx2fB from Escherichia albertii, with Uniprot accession number C6L1N1-1 (SEQ ID NO: 17). In one embodiment, a homolog of the STxB protein from Shigella dysenteriae described above is STx2fB from Escherichia albertii, with Uniprot accession number C6L1N1-1, devoid of its signal peptide. The signal peptide of STx2fB from Escherichia albertii corresponds to amino acid residues 1 to 19 of Uniprot accession number C6L1N1-1 (SEQ ID NO: 17). In one embodiment, a homolog of the STxB protein from Shigella dysenteriae described above is STx2fB from Escherichia albertii devoid of its signal peptide (SEQ ID NO: 18). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 18.

SEQ ID NO: 17
MKKMIIAVLFGLFSANSMAADCAVGKIEFSKYNEDNTFTVRVSGREYWT
NRWNLQPLLQSAQLTGMTVTIISNTCSSGSGFAQVKEN
SEQ ID NO: 18
ADCAVGKIEFSKYNEDNTFTVRVSGREYWTNRWNLQPLLQSAQLTGMTV
TIISNTCSSGSGFAQVKFN

In one embodiment, a homolog of the STxB protein from Shigella dysenteriae described above is STx2gB from Escherichia coli, with Uniprot accession number Q8VLE0-1 (SEQ ID NO: 19). In one embodiment, a homolog of the STxB protein from Shigella dysenteriae described above is STx2gB from Escherichia coli, with Uniprot accession number Q8VLE0-1, devoid of its signal peptide. The signal peptide of STx2gB from Escherichia coli corresponds to amino acid residues 1 to 19 of Uniprot accession number Q8VLE0-1 (SEQ ID NO: 19). In one embodiment, a homolog of the STxB protein from Shigella dysenteriae described above is STx2gB from Escherichia coli devoid of its signal peptide (SEQ ID NO: 20). In one embodiment, a homolog of the STxB protein from Shigella dysenteriae described above has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence set forth in SEQ ID NO: 20.

SEQ ID NO: 19
MKKMFMAVLFALVSVNAMAADCAKGKIEFSKYNGDNTFTVKVDGKEYWT
NRWNLQPLLQSAQLTGMTVTIKSNTCESGSGFAEVQFNND
SEQ ID NO: 20
ADCAKGKIEFSKYNGDNTFTVKVDGKEYWTNRWNLQPLLQSAQLTGMTV
TIKSNTCESGSGFAEVQFNND

As used herein, the term “fragment” with reference to the STxB protein or to a variant thereof refers to a portion of the STxB protein or of the variant thereof retaining the same or substantially the same biological function, activity and/or local structure, with respect to the specific biological function, activity and/or local structure identified for the full length STxB protein. A skilled person will understand that the term encompasses peptides of any origin which have a sequence corresponding to the portion of the STxB protein or of the variant thereof.

In one embodiment, a fragment of the STxB protein or of the variant thereof comprises more than 10, preferably more than 15, 20, 25, 30, 35, 40, 45, 50, 55, 60 or 65 amino acid residues, preferably consecutive, of the full length STxB protein or variant thereof.

In one embodiment, a fragment of the STxB protein or of the variant thereof comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, or 69 amino acid residues, preferably consecutive, of the full length STxB protein or variant thereof.

In one embodiment, a fragment of the STxB protein or of the variant thereof comprises more than 10, preferably more than 15, 20, 25, 30, 35, 40, 45, 50, 55, 60 or 65 amino acid residues, preferably consecutive, of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

In one embodiment, a fragment of the STxB protein or of the variant thereof comprises 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, or 69 amino acid residues, preferably consecutive, of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

In one embodiment, a fragment of the STxB protein or of the variant thereof comprises more than 10, preferably more than 15, 20, 25, 30, 35, 40, 45, 50, 55, 60 or 65 amino acid residues, preferably consecutive, of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 or SEQ ID NO: 20.

In one embodiment, a fragment of the STxB protein or of the variant thereof comprises 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, or 69 amino acid residues, preferably consecutive, of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 or SEQ ID NO: 20.

As used herein, the term “mutant” with reference to the STxB protein or to a variant thereof refers to a STxB protein or a variant thereof in which one or more amino acids have been altered (besides substitutions with, or addition of, reactive unnatural amino acid residues and/or L-norleucine described above). Such alterations include addition and/or substitution and/or deletion and/or insertion of one or several amino acid residues at the N-terminal extremity, and/or the C-terminal extremity, and/or within the amino acid sequence of the STxB protein or the variant thereof.

In one embodiment, a mutant of the STxB protein or of the variant thereof comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues which have been added, substituted, deleted or inserted, at the N-terminal extremity, and/or the C-terminal extremity, and/or within the amino acid sequence of the STxB protein or the variant thereof.

In one embodiment, a mutant of the STxB protein or of the variant thereof comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues which have been added, substituted, deleted or inserted, at the N-terminal extremity, and/or the C-terminal extremity, and/or within the amino acid sequence set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

In one embodiment, a mutant of the STxB protein or of the variant thereof comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues which have been added, substituted, deleted or inserted, at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 66, 67, 68, 69, 70, 71, 72, 73, 74, 75, 76, 77, 78, 79, 80, 81, 82, 83, 84, 85, 86, 87, 88 and/or 89 of the amino acid sequence set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19. In one embodiment, a mutant of the STxB protein or of the variant thereof comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues which have been added, substituted, deleted or inserted, at amino acid position 1, 2, 3, 4, 5, 6, 7, 8, 9, 10, 11, 12, 13, 14, 15, 16, 17, 18, 19, 20, 22, 24, 25, 27, 29, 32, 33, 34, 35, 36, 37, 38, 39, 40, 41, 42, 44, 45, 48, 49, 50, 51, 52, 53, 54, 56, 57, 58, 59, 60, 61, 62, 63, 64, 65, 66, 67, 68, 70, 71, 72, 74, 75, 76, 77, 80, 81, 82, 83, 84, 85, 86, 87 and/or 88 of the amino acid sequence set forth in SEQ ID NO: 1, or at equivalent amino acid positions in the acid sequences set forth in SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

In one embodiment, a mutant of the STxB protein or of the variant thereof comprises 1, 2, 3, 4, 5, 6, 7, 8, 9, 10, 11, 12, 13, 14, 15, 16, 17, 18, 19, 20, 21, 22, 23, 24, 25, 26 or 27 amino acid residues which have been added, substituted, deleted or inserted, at the N-terminal extremity, and/or the C-terminal extremity, and/or within the amino acid sequence set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 or SEQ ID NO: 20.

In one embodiment, a mutant of the STxB protein or of the variant thereof comprises 1, 2, 3, 4, 5, 6, 7, 8, 9, 10, 11, 12, 13, 14, 15, 16, 17, 18, 19, 20, 21, 22, 23, 24, 25, 26 or 27 amino acid residues which have been added, substituted, deleted or inserted, at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, and/or 69 of the amino acid sequence set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 or SEQ ID NO: 20. In one embodiment, a mutant of the STxB protein or of the variant thereof comprises 1, 2, 3, 4, 5, 6, 7, 8, 9, 10, 11, 12, 13, 14, 15, 16, 17, 18, 19, 20, 21, 22, 23, 24, 25, 26 or 27 amino acid residues which have been added, substituted, deleted or inserted, at amino acid position 2, 4, 5, 7, 9, 12, 13, 14, 15, 16, 17, 18, 19, 20, 21, 22, 24, 25, 28, 29, 30, 31, 32, 33, 34, 35, 36, 37, 38, 39, 40, 41, 42, 43, 44, 46, 47, 48, 50, 51, 52, 54, 56, 57, 60, 61, 62, 63, 64, 65, 66, 67, and/or 68 of the amino acid sequence set forth in SEQ ID NO: 2, or at equivalent amino acid positions in the acid sequences set forth in SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 or SEQ ID NO: 20.

In one embodiment, a mutant of the STxB protein or of the variant thereof comprises a cysteine residue which has been added or inserted at the C-terminal extremity of the STxB protein or the variant thereof.

In one embodiment, a mutant of the STxB protein or of the variant thereof comprises a cysteine residue which has been added or inserted at the C-terminal extremity of the amino acid sequence set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19.

In one embodiment, a mutant of the STxB protein or of the variant thereof comprises a cysteine residue which has been added or inserted at the C-terminal extremity of the amino acid sequence set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 or SEQ ID NO: 20.

In one embodiment, the modified monomer of the STxB protein or of the variant thereof is not a recombinant protein. In one embodiment, the modified monomer of the STxB protein or of the variant thereof is not obtained by recombinant protein production, in particular, is not obtained by a cell-based protein production system. Common cell-based protein production systems include, but are not limited to, those derived from bacteria, yeast, filamentous fungi, insect cells, and mammalian cells.

In one embodiment, the modified monomer of the STxB protein or of the variant thereof is a synthetic protein. In one embodiment, the modified monomer of the STxB protein or of the variant thereof is obtained by chemical protein synthesis.

In one embodiment, the modified monomer of the STxB protein or of the variant thereof is isolated or purified.

By “isolated” or “purified”, it is meant partially or completely extracted from its in vivo environment, whether this in vivo environment is natural (such as, e.g., the cytoplasm of a bacterium) or not (such as, e.g., a recombinant host cell culture). In particular, “isolated” or “purified” means that the modified monomer of the STxB protein or of the variant thereof is separated from, and is essentially free from association with, other molecules found in its in vivo environment (such as, e.g., other proteins, nucleic acids, and the like).

Means and methods for producing the modified STxB monomer described above are known in the art. Reference is made in particular to International patent publication WO2020245321, teaching a method of producing a monomer of a Shiga toxin B-subunit (STxB) protein or of a variant thereof by peptide chemical synthesis.

The present invention also relates to a modified oligomer of a STxB protein or of a variant thereof, comprising at least one modified monomer of the STxB protein or of the variant thereof described above.

As used herein, the term “oligomer”, when used in the context of a protein and/or polypeptide, is intended to include, but is not limited to, a protein or polypeptide structure having at least two subunits. More particularly in the context of the present invention, the term “oligomer” is intended to include a protein or polypeptide structure having at least two subunits, at least one of these subunits being a modified monomer of the STxB protein or of the variant thereof described above. In one embodiment, the at least two subunits forming the oligomer are non-covalently associated (such as, e.g., by electrostatic interactions, 7r-effects, van der Waals forces, and/or hydrophobic effects). In one embodiment, the at least two subunits forming the oligomer are covalently associated (such as, e.g., by disulfide bonds between cysteine residues of two subunits). Oligomers include, but are not limited to, dimers, trimers, tetramers, pentamers, hexamers, heptamers, octamers, nonamers, decamers and dodecamers. Greek prefixes are often used to designate the number of monomer units in the oligomer, e.g., a pentamer being composed of five units, a hexamer of six units, etc. An oligomer can further be defined as “homomer” or “heteromer”.

As used herein, the term “homomer” refers to an oligomer comprising or consisting of at least two subunits, where these at least two subunits are identical (i.e., with identical amino acid sequences and if applicable, bearing identical mutations, and if applicable, bearing identical payloads—as will be described below), and where these at least two subunits correspond to the modified monomer of the STxB protein or of the variant thereof described above.

As used herein, the term “heteromer” refers to an oligomer comprising or consisting of at least two subunits, where at least two of these at least two subunits are different (i.e., with different amino acid sequences and if applicable, bearing identical or different mutations and/or identical or different payloads—as will be described below; or with identical amino acid sequences but bearing different mutations and/or different payloads—as will be described below; or with identical amino acid sequences and bearing identical mutations but different payloads—as will be described below; or with identical amino acid sequences but bearing different mutations and identical payloads—as will be described below), and where at least one of these at least two subunits corresponds to the modified monomer of the STxB protein or of the variant thereof described above. The term “heteromer” is also intended to include an oligomer comprising or consisting of at least two subunits, where at least two of these at least two subunits are different (i.e., with different amino acid sequences, or with identical amino acid sequences but bearing different mutations), and where these at least two subunits correspond to the modified monomer of the STxB protein or of the variant thereof described above.

In one embodiment, the modified oligomer is a pentamer comprising or consisting of at least 1, preferably 2, 3, 4 or 5 modified monomers of the STxB protein or of the variant thereof described above.

In one embodiment, the modified oligomer is a homopentamer comprising or consisting of 5 modified monomers of the STxB protein or of the variant thereof described above with identical amino acid sequences and, if applicable, bearing identical mutations (in particular, identical reactive unnatural amino acid residues), and, if applicable, bearing identical payloads—as will be described below.

In one embodiment, the modified oligomer is a heteropentamer comprising or consisting of 1 modified monomer of the STxB protein or of the variant thereof described above.

In one embodiment, the modified oligomer is a heteropentamer comprising or consisting of 2 modified monomers of the STxB protein or of the variant thereof described above.

In one embodiment, the modified oligomer is a heteropentamer comprising or consisting of 3 modified monomers of the STxB protein or of the variant thereof described above.

In one embodiment, the modified oligomer is a heteropentamer comprising or consisting of 4 modified monomers of the STxB protein or of the variant thereof described above.

In one embodiment, the modified oligomer is a heteropentamer comprising or consisting of 5 modified monomers of the STxB protein or of the variant thereof described above, wherein at least 2, 3, 4 or 5 of the 5 modified monomers have different amino acid sequences and if applicable, bear identical or different mutations (in particular, identical or different reactive unnatural amino acid residues) and/or, if applicable, identical or different payloads—as will be described below.

In one embodiment, the modified oligomer is a heteropentamer comprising or consisting of 5 modified monomers of the STxB protein or of the variant thereof described above, wherein at least 2, 3, 4 or 5 of the 5 modified monomers have identical amino acid sequences but bear different mutations (in particular, different reactive unnatural amino acid residues).

In one embodiment, the modified oligomer is a heteropentamer comprising or consisting of 5 modified monomers of the STxB protein or of the variant thereof described above, wherein at least 2, 3, 4 or 5 of the 5 modified monomers have identical amino acid sequences but bear different payloads—as will be described below.

In one embodiment, the modified oligomer is a heteropentamer comprising or consisting of 5 modified monomers of the STxB protein or of the variant thereof described above, wherein at least 2, 3, 4 or 5 of the 5 modified monomers have identical amino acid sequences and bear identical mutations (in particular, identical reactive unnatural amino acid residues), but bear different payloads—as will be described below.

In one embodiment, the modified oligomer is a heteropentamer comprising or consisting of 5 modified monomers of the STxB protein or of the variant thereof described above, wherein at least 2, 3, 4 or 5 of the 5 modified monomers have identical amino acid sequences but bear different mutations (in particular, different reactive unnatural amino acid residues) and identical payloads—as will be described below.

In one embodiment, the modified oligomer retains its ability to bind to the glycosphingolipid Gb3/CD77.

As used herein, the terms “glycosphingolipid Gb3/CD77”, “Gb3”, “CD77”, “globotriaosylceramide” or “ceramide trihexoside” are used interchangeably to refer to a globoside (i.e., a type of glycosphingolipid) formed by the α-linkage of galactose to a lactosylceramide, catalyzed by lactosylceramide 4-alpha-galactosyltransferase, an enzyme encoded by the A4GALT gene (with Uniprot accession number Q9NPC4 for human A4GALT). The lactosylceramide moiety of Gb3 bears a sphingosine alkyl chain which remains, in most cases, homogenous (mainly C18:1); and a fatty acid chain which exhibits a high degree of heterogeneity among Gb3 isoforms (with different fatty acid chain length and degree of saturation from C12 to C24).

Although not fully characterized to date, Gb3 has been shown to be expressed in normal human tissues, on the cell surface of various cells, including antigen-presenting cells (APC) such as monocytes, monocyte-derived macrophages, dendritic cells and B cells (Murray et al., 1985. Int J Cancer. 36(5):561-5; Gregory et al., 1988. Int J Cancer. 42(2):213-20; Mangeney et al., 1991. Eur J Immunol. 21(5):1131-40; van Setten et al., 1996. Blood. 88(1):174-83; Falguières et al., 2001. Mol Biol Cell. 12(8):2453-68). Studies have also shown expression of Gb3 in kidney epithelium and endothelium (Lingwood, 1994. Nephron. 66(1):21-8; Khan et al., 2009. Kidney Int. 75(11):1209-1216), in microvascular endothelial cells in intestinal lamina propria (Miyamoto et al., 2006. Cell Microbiol. 8(5):869-79; Schüller et al., 2007. Microbes Infect. 9(1):35-9), in platelets (Cooling et al., 1998. Infect Immun. 66(9):4355-66), in intestinal pericryptal myofibroblasts (Schüller et al., 2007. Microbes Infect. 9(1):35-9), in neurons (Obata et al., 2008. J Infect Dis. 198(9):1398-406), and in endothelial cells in the central nervous system (Johansson et al., 2006. Cancer Biol Ther. 5(9):1211-7; Obata et al., 2008. J Infect Dis. 198(9):1398-406).

Gb3 has been further shown to be highly expressed on the surface of cancer cells in various types of cancer. To cite but a few, without limitation: fibrosarcoma (Ito et al., 1984. Int J Cancer. 34(5):689-97), Burkitt's lymphoma (Nudelman et al., 1983. Science. 220(4596):509-11), primary Burkitt-like B cell lymphomas (Nudelman et al., 1983. Science. 220(4596):509-11; Wiels et al., 1981. Proc Natl Acad Sci USA. 78(10):6485-8), other types of B cell lymphomas (Murray et al., 1985. Int J Cancer. 36(5):561-5; LaCasse et al., 1996. Blood. 88(5):1561-7; LaCasse et al., 1999. Blood. 94(8):2901-10), testicular tumor (Ohyama et al., 1990. Int J Cancer. 45(6):1040-4; Ohyama et al., 1992. J Urol. 148(1):72-5), colorectal carcinoma (Kovbasnjuk et al., 2005. Proc Natl Acad Sci USA. 102(52):19087-92; Falguières et al., 2008. Mol Cancer Ther. 7(8):2498-508; Distler et al., 2009. PLoS One. 4(8):e6813), ovary cancer (Farkas-Himsley et al., 1995. Proc Natl Acad Sci USA. 92(15):6996-7000; Arab et al., 1997. Oncol Res. 9(10):553-63), breast cancer (LaCasse et al., 1999. Blood. 94(8):2901-10; Johansson et al., 2009. BMC Cancer. 9:67), pancreatic cancer (Distler et al., 2009. PLoS One. 4(8):e6813), glioma (Arab et al., 1999. Oncol Res. 11(1):33-9; Johansson et al., 2006. Cancer Biol Ther. 5(9):1211-7), malignant meningiomas (Salhia et al., 2002. Neoplasia. 4(4):304-11), acute non-lymphocytic leukaemia (Cooling et al., 2003. Blood. 101(2):711-21).

In one embodiment, the modified oligomer retains its ability to bind to the glycosphingolipid Gb3/CD77 with a dissociation constant (K_{D}) ranging from about 10⁻⁶ M to about 10⁻¹² M, preferably from about 10⁻⁷ M to about 10⁻¹² M, from about 10⁻⁸ M to about 10⁻¹² M, from about 10⁻⁹ M to about 10⁻¹² M, from about 10⁻¹⁰ M to about 10⁻¹² M, from about 10⁻¹¹ M to about 10⁻¹² M.

In one embodiment, the modified oligomer retains its ability to bind to the glycosphingolipid Gb3/CD77 with a dissociation constant (K_{D}) ranging from about 10⁻⁶ M to about 10⁻¹¹ M, preferably from about 10⁻⁷ M to about 10⁻¹¹ M, from about 10⁻⁸ M to about 10⁻¹¹ M, from about 10⁻⁹ M to about 10⁻¹¹ M, from about 10⁻¹⁰ M to about 10⁻¹¹ M.

In one embodiment, the modified oligomer retains its ability to bind to the glycosphingolipid Gb3/CD77 with a dissociation constant (K_{D}) ranging from about 10⁻⁶ M to about 10⁻¹⁰ M, preferably from about 10⁻⁷ M to about 10⁻¹⁰ M, from about 10⁻⁸ M to about 10⁻¹⁰ M, from about 10⁻⁹ M to about 10⁻¹⁰ M.

In one embodiment, the modified oligomer retains its ability to bind to the glycosphingolipid Gb3/CD77 with a dissociation constant (K_{D}) ranging from about 10⁻⁶ M to about 10⁻⁹ M, preferably from about 10⁻⁷ M to about 10⁻⁹ M, from about 10⁻⁸ M to about 10⁻⁹ M.

In one embodiment, the modified oligomer retains its ability to bind to the glycosphingolipid Gb3/CD77 with a dissociation constant (K_{D}) of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about nM, about 75 nM, about 80 nM, about 85 nM, about 90 nM, about 95 nM, about 100 nM, about 125 nM, about 150 nM, about 175 nM, about 200 nM, about 225 nM, about 250 nM, about 275 nM, about 300 nM, about 325 nM, about 350 nM, about 375 nM, about 400 nM, about 425 nM, about 450 nM, about 475 nM, about 500 nM, about 525 nM, about 550 nM, about 575 nM, about 600 nM, about 625 nM, about 650 nM, about 675 nM, about 700 nM, about 725 nM, about 750 nM, about 775 nM, about 800 nM, about 825 nM, about 850 nM, about 875 nM, about 900 nM, about 925 nM, about 950 nM, about 975 nM, or about 1 μM.

Techniques for determining the dissociation constant (K_{D}) of the oligomer according to the present invention binding to Gb3 are well known by the skilled artisan, and include, without limitation, enzyme linked immunosorbent assays (ELISA), surface plasmon resonance (SPR), isothermal titration calorimetry (ITC), biolayer interferometry (BLI), affinity capillary electrophoresis (ACE), electrophoretic mobility shift assay (EMSA), gel-shift assays, pull-down assays, equilibrium dialysis, analytical ultracentrifugation, spectroscopic assays, and the like.

Means and methods for producing the modified STxB oligomer described above are known in the art. Reference is made in particular to International patent publication WO2020245321, teaching a method of producing a pentamer of a Shiga toxin B-subunit (STxB) protein or of a variant thereof by peptide chemical synthesis.

The present invention also relates to STxB monomer conjugates, comprising a monomer of the STxB protein or of the variant thereof, and a payload.

As used herein, the term “conjugate” refers to a chimeric STxB protein or variant thereof which is bound to a payload, optionally through a linker, thereby forming a single molecule.

In one embodiment, the STxB monomer conjugate comprises a monomer of the STxB protein or of the variant thereof, bound to a payload, optionally through a linker, at one or several, such as 1, 2, 3, 4, 5, 6, 7, 8, 9, 10, 11 or 12, amino acid positions selected from the group comprising or consisting of the C-terminal extremity (i.e., after Arg 69), Asp 3, Lys 8, Glu 10, Tyr 11, Lys 23, Lys 27, Thr 49, Lys 53, His 58, Asn 59, and Arg 69, reference made to SEQ ID NO: 2 numbering, or at one or several equivalent positions in a variant thereof.

In one embodiment, the STxB monomer conjugate comprises a monomer of the STxB protein or of the variant thereof, bound to a payload, optionally through a linker, at one or several, such as 1, 2, 3, 4, 5, 6, 7, 8, 9, 10 or 11, amino acid positions selected from the group comprising or consisting of the C-terminal extremity (i.e., after Arg 69), Asp 3, Glu 10, Tyr 11, Lys 23, Lys 27, Thr 49, Lys 53, His 58, Asn 59, and Arg 69, reference made to SEQ ID NO: 2 numbering, or at one or several equivalent positions in a variant thereof.

In one embodiment, the STxB monomer conjugate comprises a monomer of the STxB protein or of the variant thereof, bound to a payload, optionally through a linker, at the C-terminal extremity (i.e., after Arg 69), reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at one or several, such as 1, 2, 3, 4, 5, 6, 7, 8, 9, 10 or 11, amino acid positions selected from the group comprising or consisting of Asp 3, Lys 8, Glu 10, Tyr 11, Lys 23, Lys 27, Thr 49, Lys 53, His 58, Asn 59 and Arg 69, reference made to SEQ ID NO: 2 numbering, or at one or several equivalent positions in a variant thereof.

In one embodiment, the STxB monomer conjugate comprises a monomer of the STxB protein or of the variant thereof, bound to a payload, optionally through a linker, at one or several, such as 1, 2, 3, 4, 5, 6, 7, 8, 9, 10 or 11, amino acid positions selected from the group comprising or consisting of Asp 3, Glu 10, Tyr 11, Lys 23, Lys 27, Thr 49, Lys 53, His 58, Asn 59 and Arg 69, reference made to SEQ ID NO: 2 numbering, or at one or several equivalent positions in a variant thereof.

In one embodiment, the STxB monomer conjugate comprises a monomer of the STxB protein or of the variant thereof, bound to a payload, optionally through a linker, at one amino acid position selected from the group comprising or consisting of Asp 3, Lys 8, Glu 10, Tyr 11, Lys 23, Lys 27, Thr 49, Lys 53, His 58, Asn 59, Arg 69 and the C-terminal extremity (i.e., after Arg 69), reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at one amino acid position selected from the group comprising or consisting of Asp 3, Glu 10, Tyr 11, Lys 23, Lys 27, Thr 49, Lys 53, His 58, Asn 59, Arg 69 and the C-terminal extremity (i.e., after Arg 69), reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at one amino acid position selected from the group comprising or consisting of Asp 3, Lys 8, Glu 10, Tyr 11, Lys 23, Lys 27, Thr 49, Lys 53, His 58, Asn 59 and Arg 69, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at one amino acid position selected from the group comprising or consisting of Asp 3, Glu 10, Tyr 11, Lys 23, Lys 27, Thr 49, Lys 53, His 58, Asn 59 and Arg 69, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Asp 3, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Lys 8, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Glu 10, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Tyr 11, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Lys 23, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Lys 27, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Thr 49, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Lys 53, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position His 58, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Asn 59, reference made to SEQ ID NO: 2 numbering, or at one equivalent position in a variant thereof.

In one embodiment, the STxB monomer conjugate comprises a monomer of the STxB protein or of the variant thereof, bound to a payload, optionally through a linker, at amino acid position Arg 69, reference made to SEQ ID NO: 2 numbering, or at one equivalent position in a variant thereof.

In one embodiment, the STxB monomer conjugate does not comprise a monomer of the STxB protein or of the variant thereof, bound to a payload, optionally through a linker, at one or several amino acid positions selected from the group comprising or consisting of Thr 1, Thr 6 and Asp 26, according to SEQ ID NO: 2 numbering, or at one or several equivalent positions in a variant thereof.

In one embodiment, the STxB monomer conjugate does not comprise a monomer of the STxB protein or of the variant thereof, bound to a payload, optionally through a linker, at amino acid position Thr 1, according to SEQ ID NO: 2 numbering, or at one equivalent position in a variant thereof.

In one embodiment, the STxB monomer conjugate does not comprise a monomer of the STxB protein or of the variant thereof, bound to a payload, optionally through a linker, at amino acid position Thr 6, according to SEQ ID NO: 2 numbering, or at one equivalent position in a variant thereof.

In one embodiment, the STxB monomer conjugate does not comprise a monomer of the STxB protein or of the variant thereof, bound to a payload, optionally through a linker, at amino acid position Asp 26, according to SEQ ID NO: 2 numbering, or at one equivalent position in a variant thereof.

In one embodiment, the STxB monomer conjugate does not comprise a monomer of the STxB protein or of the variant thereof, bound to a payload, optionally through a linker, at amino acid positions Thr 1, Thr 6 and Asp 26, according to SEQ ID NO: 2 numbering, or at equivalent positions in a variant thereof.

Examples of suitable payloads include, but are not limited to, peptides, polypeptides, proteins, polymers, nucleic acid molecules, small molecules, mimetic agents, synthetic drugs, inorganic molecules, organic molecules and radioisotopes.

Alternatively or additionally, examples of suitable payloads include, but are not limited to, chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, antigens, hormones, coding or non-coding oligonucleotides, photodetectable labels, contrast agents, radiolabels, and the like.

It will be apparent that some payloads may fall into more than one category.

In one embodiment, the payload is a chemotherapeutic agent.

As used herein, the term “chemotherapeutic agent” refers to any molecule that is effective in inhibiting tumor growth.

Suitable examples of chemotherapeutic agents include those described under subgroup L01 of the Anatomical Therapeutic Chemical Classification System.

Suitable examples of chemotherapeutic agents include, but are not limited to:
alkylating agents, such as, e.g.;
   nitrogen mustards, including chlormethine, cyclophosphamide, ifosfamide, trofosfamide, chlorambucil, melphalan, prednimustine, bendamustine, uramustine, chlornaphazine, cholophosphamide, estrarnustine, mechlorethamine, mechlorethamine oxide hydrochloride, novembichin, phenesterine, uracil mustard and the like;
   nitrosoureas, including carmustine, lomustine, semustine, fotemustine, nimustine, ranimustine, streptozocin, chlorozotocin, and the like;
   alkyl sulfonates, including busulfan, mannosulfan, treosulfan, and the like;
   aziridines, including carboquone, thiotepa, triaziquone, triethylenemelamine, benzodopa, meturedopa, uredopa, and the like; hydrazines, including procarbazine, and the like;
   triazenes, including dacarbazine, temozolomide, and the like; ethylenimines and methylamelamines, including altretamine, triethylenemelamine, trietylenephosphoramide, triethylenethiophosphaoramide, trimethylolomelamine and the like;
   and others, including mitobronitol, pipobroman, actinomycin, bleomycin, mitomycins (including mitomycin C, and the like), plicamycin, and the like;
acetogenins, such as, e.g., bullatacin, bullatacinone, and the like;
benzodiazepines, such as, e.g., 2-oxoquazepam, 3-hydroxyphenazepam, bromazepam, camazepam, carburazepam, chlordiazepoxide, cinazepam, cinolazepam, clonazepam, cloniprazepam, clorazepate, cyprazepam, delorazepam, demoxepam, desmethylflunitrazepam, devazepide, diazepam, diclazepam, difludiazepam, doxefazepam, elfazepam, ethyl carfluzepate, ethyl dirazepate, ethyl loflazepate, flubromazepam, fletazepam, fludiazepan, flunitrazepam, flurazepam, flutemazepam, flutoprazepam, fosazepam, gidazepam, halazepam, iclazepam, irazepine, kenazepine, ketazolam, lorazepam, lormetazepam, lufuradom, meclonazepam, medazepam, menitrazepam, metaclazepam, motrazepam, N-desalkylflurazepam, nifoxipam, nimetazepam, nitemazepam, nitrazepam, nitrazepate, nordazepam, nortetrazepam, oxazepam, phenazepam, pinazepam, pivoxazepam, prazepam, proflazepam, quazepam, QH-II-66, reclazepam, RO4491533, Ro5-4864, SH-I-048A, sulazepam, temazepam, tetrazepam, tifluadom, tolufazepam, triflunordazepam, tuclazepam, uldazepam, arfendazam, clobazam, CP-1414S, lofendazam, triflubazam, girisopam, GYKI-52466, GYKI-52895, nerisopam, talampanel, tofisopam, adinazolam, alprazolam, bromazolam, clonazolam, estazolam, flualprazolam, flubromazolam, flunitrazolam, nitrazolam, pyrazolam, triazolam, bretazenil, climazolam, EVT-201, FG-8205, flumazenil, GL-II-73, imidazenil, ¹²³I-iomazenil, L-655,708, loprazolam, midazolam, PWZ-029, remimazolam, Rol5-4513, Ro48-6791, Ro48-8684, Ro4938581, sarmazenil, SH-053-R-CH3.2′F, cloxazolam, flutazolam, haloxazolam, mexazolam, oxazolam, bentazepam, clotiazepam, brotizolam, ciclotizolam, deschloroetizolam, etizolam, fluclotizolam, israpafant, JQI, metizolam, olanzapine, telenzepine, lopirazepam, zapizolam, razobazam, ripazepam, zolazepam, zomebazam, zometapine, premazepam, clazolam, anthramycin, avizafone, rilmazafone, and the like;
antimetabolites, such as, e.g.;
   antifolates, including aminopterin, methotrexate, pemetrexed, pralatrexate, pteropterin, raltitrexed, denopterin, trimetrexate, pemetrexed, and the like;
   purine analogues, including pentostatin, cladribine, clofarabine, fludarabine, nelarabine, tioguanine, mercaptopurine, and the like;
   pyrimidine analogues, including fluorouracil, capecitabine, doxifluridine, tegafur, tegafur/gimeracil/oteracil, carmofur, floxuridine, cytarabine, gemcitabine, azacytidine, decitabine, and the like; and
   hydroxycarbamide;
androgens, such as, e.g., calusterone, dromostanolone propionate, epitiostanol, mepitiostane, testolactone, and the like;
anti-adrenals, such as, e.g., aminoglutethimide, mitotane, trilostane, and the like;
folic acid replenishers, such as, e.g., frolinic acid, and the like;
maytansinoids, such as, e.g., maytansine, ansamitocins, and the like;
platinum analogs, such as, e.g., platinum, carboplatin, cisplatin, dicycloplatin, nedaplatin, oxaliplatin, satraplatin, and the like;
antihormonal agents, such as, e.g.;
   anti-estrogens, including tamoxifen, raloxifene, aromatase inhibiting 4(5)-imidazoles, 4-hydroxytamoxifen, trioxifene, keoxifene, LY117018, onapristone, toremifene, and the like;
   anti-androgens, including flutamide, nilutamide, bicalutamide, leuprolide, goserelin, and the like;
trichothecenes, such as, e.g., T-2 toxin, verracurin A, roridinA, anguidine and the like;
toxoids, such as, e.g., cabazitaxel, docetaxel, larotaxel, ortataxel, paclitaxel, tesetaxel, and the like;
others, such as, e.g., camptothecin (including the synthetic analogue topotecan); bryostatin; callystatin: CC-1065 (including its adozelesin, carzelesin and bizelesin synthetic analogues): cryptophycins (including cryptophycin 1 and cryptophycin 8); dolastatin: duocarmycin (including its synthetic analogues KW-2189 and CBI-TMD: eleutherobin; pancratistatin; sarcodictyin; spongistatin; aclacinomysins; authramycin; azaserine; bleomycin; cactinomycin; carabicin: canninomycin; carzinophilin; chromomycins: dactinomycin; daunorubicin; detorubicin; 6-diazo-5-oxo-L-norleucine; doxorubicin (including morpholino-doxorubicin, cyanomorpholino-doxorubicin, 2-pyrrolino-doxorubicin, deoxydoxorubicin, and the like); epirubicin; esorubicin; idarubicin; marcellomycin; mycophenolic acid; nogalarnycin; olivomycins; peplomycin; potfiromycin; puromycin; quelamycin; rodorubicin: streptomgrin; streptozocin; tubercidin; ubenimex: zinostatin; zorubicin; aceglatone; aldophosphamide glycoside; aminolevulinic acid; amsacrine; bestrabucil; bisantrene; edatraxate; defofamine: demecolcine; diaziquone; elfornithine: elliptinium acetate; epothilone; etoglucid; gallium nitrate; hydroxyurea; lentinan; lonidamine; mitoguazone: mitoxantrone; mopidamol; nitracrine; phenamet: pirarubicin; podophyllinic acid; 2-ethylhydrazide; PSK®; razoxane; rhizoxin: sizofiran; spirogennanium; tenuazonic acid; 2,2′,2″-trichlomtriethylamine: urethan; vindesine; dacarbazine: mannomustine; mitobromtol: mitolactol: pipobroman; gacytosine: arabinoside; 6-thioguanine; vinblastine; etoposide; vincristine; vinorelbine; navelbine; novantrone: teniposide; daunomycin; xeloda; ibandronate; CPT-11; topoisomerase inhibitor RFS 2000; topoisomerase I inhibitor SN38; difluoromethylornithine; retinoic acid; and the like.

In one embodiment, the payload is a targeted therapy agent.

As used herein, the term “targeted therapy agent” refers to any molecule which aims at one or more particular target molecules (such as, e.g., proteins) involved in tumor genesis, tumor progression, tumor metastasis, tumor cell proliferation, cell repair, and the like.

Suitable examples of targeted therapy agents include, but are not limited to, tyrosine-kinase inhibitors, serine/threonine kinase inhibitors, monoclonal antibodies and the like.

Suitable examples of targeted therapy agents include, but are not limited to, HER1/EGFR inhibitors (such as, e.g., brigatinib, erlotinib, gefitinib, olmutinib, osimertinib, rociletinib, vandetanib, and the like): HER2/neu inhibitors (such as, e.g., afatinib, lapatinib, neratinib, and the like); C-kit and PDGFR inhibitors (such as, e.g., axitinib, masitinib, pazopanib, sunitinib, sorafenib, toceranib, and the like); FLT3 inhibitors (such as, e.g., lestaurtinib, and the like); VEGFR inhibitors (such as, e.g., axitinib, cediranib, lenvatinib, nintedanib, pazopanib, regorafenib, semaxanib, sorafenib, sunitinib, tivozanib, toceranib, vandetanib, and the like): RET inhibitors (such as, e.g., vandetanib, entrectinib, and the like); c-MET inhibitors (such as, e.g., cabozantinib, and the like): bcr-abl inhibitors (such as, e.g., imatinib, dasatinib, nilotinib, ponatinib, radotinib, and the like): Src inhibitors (such as, e.g., bosutinib, dasatinib, and the like); Janus kinase inhibitors (such as, e.g., lestaurtinib, momelotinib, ruxolitinib, pacritinib, and the like): MAP2K inhibitors (such as, e.g., cobimetinib, selumetinib, trametinib, binimetinib, and the like); EML4-ALK inhibitors (such as, e.g., alectinib, brigatinib, ceritinib, crizotinib, and the like); Bruton's inhibitors (such as, e.g., ibrutinib, and the like); mTOR inhibitors (such as, e.g., everolimus, temsirolimus, and the like); hedgehog inhibitors (such as, e.g., sonidegib, vismodegib, and the like); CDK inhibitors (such as, e.g., palbociclib, ribociclib, and the like); anti-HER1/EGFR monoclonal antibodies (such as, e.g., cetuximab, necitumumab, panitumumab, and the like): anti-HER2/neu monoclonal antibodies (such as, e.g., ado-trastuzumab emtansine, pertuzumab, trastuzumab, trastuzumab-dkst, and the like); anti-EpCAM monoclonal antibodies (such as, e.g., catumaxomab, edrecolomab, and the like); anti-VEGF monoclonal antibodies (such as, e.g., bevacizumab, bevacizumab-awwb, and the like); anti-CD20 monoclonal antibodies (such as, e.g., ibritumomab, obinutuzumab, ocrelizumab, ofatumumab, rituximab, tositumomab, and the like); anti-CD30 monoclonal antibodies (such as, e.g., brentuximab, and the like); anti-CD33 monoclonal antibodies (such as, e.g., gemtuzumab, and the like); and anti-CD52 monoclonal antibodies (such as, e.g., alemtuzumab, and the like).

In one embodiment, the payload is a cytotoxic agent.

As used herein, the term “cytotoxic agent” refers to any molecule that results in cell death by any mechanism.

Suitable examples of cytotoxic agents include, but are not limited to, taxanes, anthracyclines, alkylating agents, vinca alkaloids, antimetabolites, platinum agents, steroids, and chemotherapeutic agents.

Suitable examples of taxanes include, but are not limited to, cabazitaxel, docetaxel, larotaxel, ortataxel, paclitaxel and tesetaxel.

Suitable examples of anthracyclines include, but are not limited to, aclarubicin, amrubicin, daunorubicin, doxorubicin, epirubicin, idarubicin, pirarubicin, valrubicin and zorubicin.

Suitable examples of alkylating agent include, but are not limited to, nitrogen mustards (such as, e.g., chlormethine, cyclophosphamide, ifosfamide, trofosfamide, chlorambucil, melphalan, prednimustine, bendamustine, uramustine, and the like), nitrosoureas (such as, e.g., carmustine, lomustine, semustine, fotemustine, nimustine, ranimustine, streptozocin, and the like), alkyl sulfonates (such as, e.g., busulfan, mannosulfan, treosulfan, and the like), aziridines (such as, e.g., carboquone, thiotepa, triaziquone, triethylenemelamine, benzodopa, meturedopa, uredopa, and the like), hydrazines (such as, e.g., procarbazine, and the like), triazenes (such as, e.g., dacarbazine, temozolomide, and the like), altretamine, mitobronitol, pipobroman, actinomycin, bleomycin, mitomycins and plicamycin.

Suitable examples of vinca alkaloids include, but are not limited to, vinblastine, vincristine, vinflunine, vindesine and vinorelbine.

Suitable examples of antimetabolites include, but are not limited to, antifolates (such as, aminopterin, methotrexate, pemetrexed, pralatrexate, raltitrexed, pemetrexed, and the like), purine analogues (such as, e.g., pentostatin, cladribine, clofarabine, fludarabine, nelarabine, tioguanine, mercaptopurine, and the like), pyrimidine analogues (such as, e.g., fluorouracil, capecitabine, doxifluridine, tegafur, tegafur/gimeracil/oteracil, carmofur, floxuridine, cytarabine, gemcitabine, azacytidine, decitabine, and the like), and hydroxycarbamide.

Suitable examples of platinum agents include, but are not limited to, carboplatin, cisplatin, dicycloplatin, nedaplatin, oxaliplatin and satraplatin.

Suitable examples of steroids include, but are not limited to, estrogen receptor modulators, androgen receptor modulators and progesterone receptor modulators.

Suitable examples of chemotherapeutic agents have been described above.

In one embodiment, the payload is an antibiotic.

Suitable examples of antibiotics include those described under subgroup J01 of the Anatomical Therapeutic Chemical Classification System.

Suitable examples of antibiotics include, but are not limited to:
aminoglycosides, such as, e.g., amikacin, gentamicin, kanamycin, neomycin, netilmicin, streptomycin, tobramycin, paromycin, and the like;
ansamycins, such as, e.g., geldanamycin, herbimycin and the like;
carbacephems, such as, e.g., loracarbef and the like;
carbapenems, such as, e.g., ertapenem, doripenem, imipenem, cilastatin, meropenem, and the like;
first generation cephalosporins, such as, e.g., cefadroxil, cefazolin, cefalotin, cephalexin, and the like;
second generation cephalosporins, such as, e.g., ceflaclor, cefamandole, cefoxitin, cefprozil, cefuroxime, and the like;
third generation cephalosporins, such as, e.g., cefixime, cefdinir, cefditoren, cefoperazone, cefotaxime, cefpodoxime, ceftazidime, ceftibuten, ceftizoxime, ceftriaxone, and the like;
fourth generation cephalosporins, such as, e.g., cefepime and the like;
fifth generation cephalosporins, such as, e.g., ceftobiprole, and the like;
glycopeptides, such as, e.g., teicoplanin, vancomycin, and the like;
macrolides, such as, e.g., azithromycin, clarithromycin, dirithromycine, erythromycin, roxithromycin, troleandomycin, telithromycin, spectinomycin, and the like;
monobactams, such as, e.g., aztreonam, and the like;
penicillins, such as, e.g., amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, and the like;
antibiotic polypeptides, such as, e.g., bacitracin, colistin, polymyxin B, and the like;
quinolones, such as, e.g., ciprofloxacin, enoxacin, gatifloxacin, levofloxacin, lomefloxacin, moxifloxacin, norfloxacin, orfloxacin, trovafloxacin, and the like;
sulfonamides, such as, e.g., mafenide, prontosil, sulfacetamide, sulfamethizole, sulfanilamide, sulfasalazine, sulfisoxazole, trimethoprim, trimethoprim-sulfamethoxazole, and the like;
tetracyclines, such as, e.g., demeclocycline, doxycycline, minocycline, oxytetracycline, tetracycline, and the like; and
others such as, e.g., arspenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin, tinidazole, and the like.

In one embodiment, the payload is an antiviral.

Suitable examples of antivirals include those described under subgroup J05 of the Anatomical Therapeutic Chemical Classification System.

Suitable examples of antivirals include, but are not limited to, acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, zinc salts, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabine, zidovudine, and zinviroxime.

In one embodiment, the payload is a cell cycle-synchronizing agent.

As used herein, the term “cell cycle-synchronizing agent” refers to any molecule able to for unify the cell cycle of a population of cells to the same phase upon administration.

Suitable examples of cell cycle-synchronizing agents include, but are not limited to, aphidicolin, butyrolactone I, colchicine, cycloheximide, demecolcine, dimethyl sulfoxide, 5-fluorodeoxyuridine, Hoechst 33342, mimosine, nocodazole, roscovitine, and thymidine.

In one embodiment, the payload is a ligand for a cellular receptor.

As used herein, the term “ligand for a cellular receptor” refers to any molecule binding to a cellular receptor (such as a cell surface receptor, an intracellular receptor or a co-receptor, including transcription factors and the like), including agonists and antagonists, as well as partial agonists, inverse agonists, and allosteric modulators.

Suitable examples of ligands for cellular receptors include, but are not limited to, ligands binding to the AATYK receptors, the acetylcholine receptors, the ADGRG receptors, the adiponectin receptors, the adrenergic α1 receptors, the adrenergic α2 receptors, the adrenergic β1 receptors, the adrenergic β2 receptors, the adrenergic β3 receptors, the adrenomedullin receptor, the AMPA receptors, the anaphylatoxin receptors, the angiopoietin receptors, the angiotensin receptors, the anti-Müllerian hormone receptor, the apelin receptor, the asialoglycoprotein receptors, the AXL receptors, the benzodiazepine receptor, the bile acid receptor, the bombesin receptors, the bone morphogenetic protein receptors, the bradykinin receptors, the brain-specific angiogenesis inhibitors, the cadherin receptors, the calcitonin receptor, the calcitonin receptor-like receptor, the calcium-sensing receptor, the cannabinoid receptors, the CD97 receptor, the chemokine receptors, the cholecystokinin receptors, the complement receptors, the corticotropin-releasing hormone receptors, the CysLT receptors, the cytokine receptors, the DDR receptors, the dopamine receptors, the EB12 receptor, the ectodysplasin A receptor, the EGF module-containing mucin-like hormone receptors, the EGF receptors, the endothelin receptors, the EPH receptors, the estrogen receptor, the FGF receptors, the free fatty acid receptors, the frizzled receptors, the FSH receptor, the GABAB receptors, the galanin receptors, the GHB receptor, the ghrelin receptor, the glucagon receptors, the glucagon-like peptide receptors, the glutamate receptors, the glycine receptors, the gonadotropin receptors, the gonadotropin-releasing hormone receptors, the GPRC6A receptor, the growth factor receptors, the growth hormone receptors, the growth-hormone-releasing hormone receptor, the guanylate cyclase-coupled receptors, the HGF receptors, the histamine receptors, the hydroxycarboxylic acids receptors, the immunoglobulin immune receptors, the insulin receptors, the kainate receptors, the KiSS1-derived peptide receptor, the latrophilin receptors, the leptin receptor, the leukotriene B4 receptors, the lipoprotein receptor-related protein receptors, the LTK receptors, the luteinizing hormone/choriogonadotropin receptor, the lysophosphatidic acid receptors, the lysophospholipid receptors, the mannose receptor, the MAS receptors, the melanin-concentrating hormone receptors, the melanocortin receptors, the melatonin receptors, the methuselah-like proteins receptors, the motilin receptor, the MuSK receptors, the N-acetylglucosamine receptor, the neuromedin receptors, the neuropeptide B/W receptors, the neuropeptide FF receptors, the neuropeptide S receptor, the neuropeptide Y receptors, the neuropilins receptor, the neurotensin receptors, the N-formyl peptide receptor, the nicotinic acetylcholine receptors, the NMDA receptors, the nuclear receptors, the olfactory receptor, the opioid receptors, the opsin receptors, the orexin receptors, the oxoeicosanoid receptor, the oxoglutarate receptor, the oxytocin receptor, the parathyroid hormone receptors, the PDGF receptors, the pituitary adenylate cyclase-activating polypeptide type I receptor, the platelet-activating factor receptor, the progestin and adipoQ receptors, the prokineticin receptors, the prolactin receptor, the prolactin-releasing peptide receptor, the prostacyclin receptor, the prostaglandin receptors, the protease-activated receptor, the PTK7 receptors, the purinergic adenosine receptors, the purinergic P2X receptors, the purinergic P2Y receptors, the relaxin receptors, the RET receptors, the retinoic acid-inducible orphan G-protein-coupled receptors, the ROR receptors, the ROS receptors, the RYK receptors, the scavenger receptors, the secretin receptor, the serine/threonine-specific protein kinase receptors, the serotonine receptors, the smoothened receptor, the somatostatin receptors, the sphingosine-1-phosphate receptors, the SREB receptors, the stimulator of interferon genes (STING) receptor, the succinate receptor, the tachykinin receptors, the thromboxane receptor, the thyrotropin receptor, the thyrotropin-releasing hormone receptor, the toll-like receptors, the trace-amine associated receptors, the transferrin receptor, the Trk receptors, the tumor necrosis factor receptors, the tyrosine phosphatase receptors, the urotensin-II receptor, the vasoactive intestinal peptide receptors, the vasoactive intestine peptide receptors, the vasopressin receptors, the VEGF receptors, the vomeronasal receptor, and the zinc-activated ion channel receptor.

In one embodiment, the payload is an immunomodulatory agent.

Suitable examples of immunomodulatory agents include, but are not limited to, immunostimulatory agents and immunosuppressor agents.

Suitable examples of immunostimulatory agents include those described under subgroup L03 of the Anatomical Therapeutic Chemical Classification System.

Suitable examples of immunostimulatory agents include, but are not limited to, cytokines (such as, e.g., filgrastim, pegfilgrastim, lenograstim, molgramostim, sargramostim, ancestim, albinterferon, interferon alfa natural, interferon alfa 2a, peginterferon alfa-2a, interferon alfa 2b, peginterferon alfa-2b, interferon alfa n1, interferon alfacon-1, interferon alpha-n3, interferon beta natural, interferon beta 1a, interferon beta 1b, interferon gamma, aldesleukin, oprelvekin, and the like); immune checkpoint inhibitors (such as, e.g., inhibitors of CTLA4, PD-1, PD-L1, LAG-3, B7-H3, B7-H4, TIM3, A2AR, and/or IDO, including nivolumab, pembrolizumab, pidilizumab, AMP-224, MPDL3280A, MDX-1105, MEDI-4736, arelumab, ipilimumab, tremelimumab, pidilizumab, IMP321, MGA271, BMS-986016, lirilumab, urelumab, PF-05082566, IPH2101, MEDI-6469, CP-870,893, mogamulizumab, varlilumab, avelumab, galiximab, AMP-514, AUNP 12, indoximod, NLG-919, INCB024360, and the like): toll-like receptor agonists (such as, e.g., buprenorphine, carbamazepine, ethanol, fentanyl. GS-9620, imiquimod, lefitolimod, levorphanol, methadone, morphine, (+)-morphine, morphine-3-glucuronide, oxcarbazepine, oxycodone, pethidine, resiquimod, SD-101, tapentadol, tilsotolimod, VTX-2337, glucuronoxylomannan from Cryptococcus, MALP-2 from Mycoplasma, MALP-404 from Mycoplasma, OspA from Borrelia, porin from Neisseria or Haemophilus, hsp60, hemaglutinin, LcrV from Yersinia, bacterial flagellin, lipopolysaccharide, lipoteichoic acid, lipomannan from Mycobacterium, glycosylphosphatidylinositol, lysophosphatidylserine, lipophosphoglycan from Leishmania, zymosan from Saccharomyces, Pam2CGDPKHPKSF, Pam3CSK4, CpG oligodeoxynucleotides, poly(I:C) nucleic acid sequences, poly(A:U) nucleic acid sequences, double-stranded viral RNA, and the like); STING receptor agonists (such as, e.g., those described in WO2017100305, vadimezan, CL656, ADU-S100, 3′3′-cGAMP, 2′3′-cGAMP, ML RR-S2 CDG, ML RR-S2 cGAMP, cyclic di-GMP, DMXAA, DiABZI, and the like); CD1 ligands; growth hormone; immunocyanin; pegademase; prolactin; tasonermin; female sex steroids: histamine dihydrochloride; poly ICLC; vitamin D; lentinan: plerixafor; roquinimex; mifamurtide; glatiramer acetate; thymopentin; thymosin α1; thymulin; polyinosinic:polycytidylic acid; pidotimod; Bacillus Calmette-Guérin: melanoma vaccine: sipuleucel-T; and the like.

Suitable examples of immunosuppressor agents include those described under subgroup L04 of the Anatomical Therapeutic Chemical Classification System.

Suitable examples of immunosuppressor agents include, but are not limited to:
antimetabolites, such as, e.g.;
   antifolates, including aminopterin, methotrexate, pemetrexed, pralatrexate, pteropterin, raltitrexed, denopterin, trimetrexate, pemetrexed, and the like;
   purine analogues, including pentostatin, cladribine, clofarabine, fludarabine, nelarabine, tioguanine, mercaptopurine, and the like;
   pyrimidine analogues, including fluorouracil, capecitabine, doxifluridine, tegafur, tegafur/gimeracil/oteracil, carmofur, floxuridine, cytarabine, gemcitabine, azacytidine, decitabine, and the like; and
   hydroxycarbamide);
macrolides, such as, e.g., tacrolimus, ciclosporin, pimecrolimus, abetimus, gusperimus, and the like;
immunomodulatory imide drugs, such as, e.g., lenalidomide, pomalidomide, thalidomide, apremilast, and the like;
UL-1 receptor antagonists, such as, e.g., anakinra, and the like);
mTOR inhibitors, such as, e.g., sirolimus, everolimus, ridaforolimus, temsirolimus, umirolimus, zotarolimus, and the like);
serum-targeting antibodies, such as, e.g., eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, and the like;
cell-targeting antibodies, such as, e.g., muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, zolimomab aritox, atorolimumab, cedelizumab, fontolizumab, maslimomab, morolimumab, pexelizumab, reslizumab, rovelizumab, siplizumab, talizumab, telimomiab aritox, vapaliximab, vepalimomab, and the like;
fusion antibodies, such as, e.g., abatacept, belatacept, etanercept, pegsunercept, aflibercept, alefacept, rilonacept and the like.

In one embodiment, the payload is a pro-apoptotic agent.

As used herein, the term “pro-apoptotic agent” refers to any molecule able to induce apoptosis or programmed cell death in a cell upon administration.

Suitable examples of pro-apoptotic agents include, but are not limited to, histone deacetylase inhibitors (such as, e.g., sodium butyrate, depsipeptide and the like), borteiomib, deguelin, favopiridol, fenretinide, fludarabine, kaempferol, miltefosine, narciclasine, obatoclax, oblimersen, and oncrasin.

In one embodiment, the payload is an anti-angiogenic agent.

As used herein, the term “anti-angiogenic agent” refers to a molecule that reduces or prevents angiogenesis, which is responsible for the growth and development of blood vessels.

Suitable examples of anti-angiogenic agents include, but are not limited to, inhibitors of any of the vascular endothelial growth factor VEGF-A, VEGF-B, VEGF-C, or VEGF-D, which are major inducers of angiogenesis in normal and pathological conditions, and are essential in embryonic vasculogenesis.

Additionally or alternatively, an anti-angiogenic agent also can inhibit other angiogenic factors, such as, without limitation, a member of the fibroblast growth factor (FGF) family such as FGF-1 (acidic), FGF-2 (basic), FGF-4 or FGF-5; or angiopoietin-1, a factor that signals through the endothelial cell-specific Tie2 receptor tyrosine kinase; or the receptor of any of these angiogenic factors.

In one embodiment, the payload is a cytokine.

Suitable examples of cytokines include, but are not limited to, chemokines, tumor necrosis factors, interleukins, and colony-stimulating factors.

Suitable examples of chemokines include, but are not limited to, chemokine C-C motif ligand (CCL) 1, CCL2, CCL3, CCL4, CCL5, CCL6, CCL7, CCL8, CCL9, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, chemokine C-X-C motif ligand (CXCL) 1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, fractalkine, chemokine C motif ligand (XCL) 1, and XCL2.

Suitable examples of tumor necrosis factors include, but are not limited to, tumor necrosis factor (TNF) α, lymphotoxin, OX40L, CD40LG, Fas ligand, CD70, CD153, 4-1BB ligand, TNF-related apoptosis-inducing ligand (TRAIL), receptor activator of nuclear factor κ-B ligand (RANKL), a proliferation-inducing ligand (APRIL), B-cell activating factor (BAFF), and ectodysplasin A (EDA).

Suitable examples of interleukins include, but are not limited to, interleukin- (IL-) 1α, IL-1β, IL-1Ra, IL-2, IL-3, IL-4, IL-5, IL-6, IL-7, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36α, IL-36β, IL-36γ, IL-36Ra, IL-37, IL-38, interferon (IFN) α, IFNβ, IFNκ, and IFNω.

Suitable examples of colony-stimulating factors include, but are not limited to, granulocyte-macrophage colony-stimulating factor (GM-CSF) (including granulocyte-colony stimulating factor (G-CSF) and macrophage colony-stimulating factor (M-CSF)), haematopoietin, and thrombopoietin.

In one embodiment, the payload is a growth factor.

Suitable examples of growth factors include, but are not limited to, fibroblast growth factor (FGF) 1, FGF2, FGF3, FGF4, FGF5, FGF6, FGF7, FGF8, FGF9, FGF10, FGF11, FGF12, FGF13, FGF14, FGF16, FGF17, FGF18, FGF19, FGF20, FGF2I, FGF23, transforming growth factor (TGF) α, epidermal growth factor (EGF), heparin-binding EGF-like growth factor (HB-EGF), transforming growth factor (TGF) β, insulin-like growth factor (IGF) 1, IGF2, Platelet-derived growth factor (PDGF) subunit A (PDGFA), PDGF subunit B (PDGFB), PDGF subunit C (PDGFC), PDGF subunit D (PDGFD), vascular endothelial growth factor (VEGF)-A, VEGF-B, VEGF-C, VEGF-D, placental growth factor (PGF), nerve growth factor (NGF) and hepatocyte growth factor (HOF).

In one embodiment, the payload is an antibody or an antigen-binding fragment thereof.

Suitable examples of antibodies or antigen-binding fragments thereof include, but are not limited to, monoclonal antibodies, polyclonal antibodies, bispecific antibodies, multispecific antibodies, antibody fragments, and antibody mimetics, such as, e.g., scFv, di-scFv, tri-scFv, single domain antibodies, nanobodies, bispecific T-cell engagers (BiTEs), Fab, F(ab′)2. Fab′, chemically linked Fab, X-Link Fab, tandem-scFv/BiTE, diabodies, tandem diabodies, diabody-Fc fusions, tandem diabody-Fe fusion, tandem diabody-CH3 fusion, tetra scFv-Fc fusion, dual variable domain immunoglobulin, knob-hole, strand exchange engineered domain, CrossMab, quadroma-derived bispecific antibody, single domain based antibody, affibodies, affilins, affimers, affitins, alphabodies, anticalins, avimer, DARPins, Kunitz domain peptides, monobodies and nanoCLAMPs.

In one embodiment, the payload is an antigen.

As used herein, the term “antigen”, also termed “immunogen”, refers to any substance that induces a state of sensitivity and/or immune responsiveness after any latent period (normally, days to weeks in humans) and that reacts in a demonstrable way with antibodies and/or immune cells of the sensitized subject in vivo or in vitro.

Suitable examples of antigens include, but are not limited to, pathogen-related antigens (such as, e.g., antigens of viruses, fungi or bacteria, or immunogenic molecules derived from them), self-antigens (such as, e.g., cellular antigens including cells containing normal transplantation antigens and/or tumor-related antigens, RR-Rh antigens, and antigens characteristic of, or specific to particular cells or tissues or body fluids), allergen-related antigens (such as, e.g., those associated with environmental allergens, including grasses, pollens, molds, dust, insects, dander, venoms, and the like; occupational allergens, including latex, dander, urethanes, epoxy resins, and the like; food, including shellfish, peanuts, eggs, milk products, and the like; and drugs, including antibiotics, anesthetics, and the like), and vaccines.

Suitable examples of pathogen-related antigens include, but are not limited to, antigens derived from vaccinia, avipox virus, turkey influenza virus, bovine leukemia virus, feline leukemia virus, avian influenza, chicken pneumovirosis virus, canine parvovirus, equine influenza, FHV, Newcastle Disease Virus (NDV), Chicken/Pennsylvania/l/83 influenza virus, infectious bronchitis virus, Dengue virus, measles virus, Rubella virus, pseudorabies, Epstein-Barr Virus, HIV, SIV, EHV, BHV, HCMV, Hantaan, C. tetani, mumps, Morbillivirus, Herpes Simplex Virus type 1, Herpes Simplex Virus type 2, Human cytomegalovirus, Hepatitis A Virus, Hepatitis B Virus, Hepatitis C Virus, Hepatitis E Virus, Respiratory Syncytial Virus, Human Papilloma Virus, Influenza Virus, Salmonella, Neisseria, Borrelia, Chlamydia, Bordetella, Plasmodium, Toxoplasma, Cryptococcus, Streptococcus, Staphylococcus, Haemophilus, Diptheria, Tetanus, Pertussis, Escherichia, Candida, Aspergillus, Entamoeba, Giardia, and Trypanosoma.

Suitable examples of self-antigens include, but are not limited to, lupus autoantigen, Smith, Ro, La, U1-RNP, fibrillin, nuclear antigens, histones, glycoprotein gp70, ribosomal proteins, pyruvate dehydrogenase, dehydrolipoamide acetyltransferase (PCD-E2), hair follicle antigens, human tropomyosin isoform 5 (hTM5), proinsulin, insulin, IA2, GAD65, collagen type II, human cartilage gp 39 (HCgp39), gp130-RAPS, dnaJp1, citrullinated proteins and peptides (including citrullinated type II collagen, citrullinated vimentin and citrullinated fibrinogen), myelin basic protein, proteolipid protein (PLP), myelin oligodendrocyte glycoprotein (MOG), thyroid stimulating factor receptor (TSH-R), acetylcholine receptor (AchR), gliadin, PLP, glucose-6-phosphate isomerase, thyroglobulin, various tRNA synthetases, proteinase-3, and myeloperoxidase, and the like, including fragments thereof.

Suitable examples of tumor-related antigens include, but are not limited to, MART-1/Melan-A, gplOO, dipeptidyl peptidase IV (DPPIV), adenosine deaminase-binding protein (ADAbp), cyclophilin b, colorectal associated antigen (CRC)-C017-1A/GA733, carcinoembryonic antigen (CEA) and its immunogenic epitopes CAP-1 and CAP-2, etv6, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family (e.g. MUC1, MUC16, etc.), HER2/neu, p21ras, RCAS1, alpha-fetoprotein, E-cadherin, alpha-catenin, beta-catenin and gamma-catenin, pl20ctn, gp100.sup.Pmell17, PRAME, NY-ESO-1, cdc27, adenomatous polyposis coli protein (APC), fodrin, connexin 37, Ig-idiotype, p15, gp75, GM2 and GD2 gangliosides, Smad family of cancer antigens brain glycogen phosphorylase, SSX-1, SSX-2 (HOM-MEL-40), SSX-1, SSX-4, SSX-5, SCP-1 and CT-7, and c-erbB-2 and viral antigens such as the HPV-16 and HPV-18 E6 and E7 antigens and the EBV-encoded nuclear antigen (EBNA)-1, and the like, including fragments thereof. Further examples of tumor-related antigens are described in, e.g., Li et al., 2004. Cancer Immunol Immunother. 53(3):139-43; Novellino et al., 2005. Cancer Immunol Immunother. 54(3):187-20; which are herein incorporated by reference in their entirety.

In one embodiment, the payload is a hormone.

Suitable examples of hormones include, but are not limited to, GnRH, TRI-H, dopamine, CRH, GHRH, somatostatin, MCH, oxytocin, vasopressin, FSH, LH, TSH, prolactin, POMC, CLIP, ACTH, MSH, endorphins, lipotropin, GH, aldosterone, cortisol, cortisone, DHEA, DHEA-S, androstenedione, epinephrine, norepinephrine, thyroid hormone T3, thyroid hormone T4, calcitonin, PTH, testosterone, AMH, inhibin, estradiol, progesterone, activin, relaxin, GnSAF, hCG, HPL, estrogen, glucagon, insulin, amylin, pancreatic polypeptide, melatonin, N,N-dimethyltryptamine, 5-methoxy-N,N-dimethyltryptamine, thymosin α1, beta thymosins, thymopoietin, thymulin, gastrin, ghrelin, CCK, GIP, GLP-1, secretin, motilin, VIP, enteroglucagon, peptide YY, IGF-1, IGF-2, leptin, adiponectin, resistin, osteocalcin, renin, EPO, calcitriol, prostaglandin, ANP, and BNP.

In one embodiment, the payload is a coding or non-coding oligonucleotide.

Suitable examples of coding or non-coding oligonucleotides include, but are not limited to, messenger RNA (mRNA), antisense RNA (asRNA), small interfering RNA (siRNA), microRNA (miRNA), long non-coding RNA (lncRNA) (such as, e.g., transfer RNA [tRNA], ribosomal RNA [rRNA], and the like), small temporal RNA (stRNA), trans-acting siRNA, short hairpin RNA (shRNA), cis-natural antisense transcripts (NATs), CRISPR RNA, long noncoding RNA, piwi-interacting RNA (piRNA), repeat-associated siRNA (rasiRNA), RNA aptamers, ribozymes, and the like.

Further suitable examples of coding or non-coding oligonucleotides include, but are not limited to, recapuldencel-T, TriMix, BI-1361849, nusinersen, volanesorsen sodium, eteplirsen, ATL1105, ASM-8, inclisiran, patisiran, RXI-109, fitusiran, cemdisiran, QPI-1002, BMS-986263, PF-655, pegaptanib, avacincaptad pegol sodium, olaptesed pegol, emapticap pegol, SPC3649, bevasiranib, AGN-745, QPI-1007, TD101, SYL040012, SYL1001, Excellair, ALN-RSV01, CEQ508, siG12D LODER, TKM-ApoB, TKM-PLK1, ALN-VSP02, ALN-TTR01, Bcr-Abl siRNA, Atu027, 15NP, CALAA-01, FANG vaccine, iPsiRNA, Tat/Rev shRNA, ARC1779, ARC19499, AS1411 (AGRO001), Fovista, NOX-A12, NOX-E36, NOX-194, NU172, RB006 plus RB007, ARC1905, as well as those described in Table 1 and Table 2 of Crooke el al., 2018 (Cell Metab. 27(4):714-739), herein incorporated by reference.

In one embodiment, the payload is a photodetectable label.

As used herein, the terms “photodetectable label” or “fluorophore” refer to a moiety that can re-emit light upon light excitation.

Suitable examples of photodetectable labels include, but are not limited to, Alexa Fluor® dyes, BODIPY® dyes, fluorescein, 5-carboxyfluorescein, 5-(4,6-dichlorotriazin-2-yl) aminofluorescein, 2′7′-dimethoxy-4′5′-dichloro-6-carboxyfluorescein, fluorescein isothiocyanate (FITC), QFITC, Oregon Green® 488, Oregon Green® 514, rhodamine and derivatives thereof (such as, e.g., rhodamine green, rhodamine green-X, rhodamine red-X, X-rhodamine, 6-carboxy-X-rhodamine (ROX), 6-carboxyrhodamine (R6G), N,N,N′,N′-tetramethyl-6-carboxyrhodamine (TAMRA), lissamine rhodamine B, rhodamine 123, rhodamine X isothiocyanate, sulforhodamine B, sulforhodamine 101 (Texas Red), tetramethyl rhodamine, tetramethyl rhodamine isothiocyanate (TRITC)), eosin, eosin isothiocyanate, erythrosine, erythrosine B, erythrosin isothiocyanate, Texas Red®, Texas Red®-X, naphthofluorescein, malachite green, malachite green isothiocyanate, coumarin derivatives, Pacific Orange, cascade blue, cascade yellow, dansyl chloride, dapoxyl dye, 1-dimethylamine-N(2-azido-ethyl)naphthalene-5-sulfonamide, 6-(6-amino-2-(2-azidoethyl)-1,3-dioxo-1H-benzo(de)-2(3H)isoquinoline, 6-(6-amino-2-(2-propinyl)-1,3-dioxo-1H-benzo(de)-2(3H)isoquinoline, 8-(4-azidoethyloxyphenyl)-2,6-diethyl-1,3,5,7-tetramethyl-4,4-difluoro-4-bora-3a,4a-diaza-s-indacene, 8-(4-propionyloxyphenyl)-2,6-diethyl-1,3,5,7-tetramethyl-4,4-difluoro-4-bora-3a,4a-diaza-s-indacene, 1-(3-azido-propoxy)-7-methylamino-phenoxazin-3-one, 1-(2-propynyl)-7-methylamino-phenoxazm-3-one, N-(5-(3-azidopropylamino)-9H-benzo(a)-phenoxa-2-in-9-ylidene)-N-methyl-methanaminium chloride, N-(5-(3-propynyl-amino)-9H-benzo(a)-phenoxazin-9-ylene)-N-methyl-methanaminium chloride, (9-(3-azido-propoxy)-7-piperidin-1-yl-phenoxazin-3-ylidene)-dimethyl-ammonium perchlorate, 4-acetamido-4′-isothiocyanatostilbene-2,2′-disulfonic acid, acridine, acridine isothiocyanate, 5-(2′-aminoethyl)aminonaphthalene-1-sulfonic acid, 4-amino-N-[3-vinylsulfonyl)phenyl]naphthalimide-3,5 disulfonate, N-(4-anilino-1-naphthyl)maleimide, anthranilamide, Brilliant Yellow, coumarin, coumarin derivatives, 7-amino-4-methylcoumarin, 7-amino-trifluoromethylcouluarin, cyanosine, 4′,6-diaminidino-2-phenylindole, 5′,5″-dibromopyrogallol-sulfonephthalein, 7-diethylamino-3-(4′-isothiocyanatophenyl)-4-methylcoumarin-4,4′-diisothiocyanatodihydro-stilbene-2,2′-disulfonic acid, 4,4′-diisothiocyanatostilbene-2,2′-disulfonic acid, ethidium, IR144, IR1446, 4-methylumbelliferone, o-cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4, riboflavin, rosolic acid, lanthanide chelates, quantum dots, cyanines, pyrelium dyes, and squaraines.

In one embodiment, the payload is a contrast agent.

As used herein, the term “contrast agent” refers to any molecule used to increase the contrast of structures or fluids within the body in medical imaging. Contrast agents absorb or alter external electromagnetism or ultrasound (which differs from radiolabels which emit radiation themselves).

Suitable examples of radiolabels include those described under subgroup V08 of the Anatomical Therapeutic Chemical Classification System.

Suitable examples of contrast agents include, but are not limited to, diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, calcium iopodate, iopydol, propyliodone, iofendylate, lipiodol, barium sulfate, gadobenic acid, gadobutrol, gadodiamide, gadofosveset, gadolinium, gadopentetic acid, gadoteric acid, gadoleridol, gadoversetamide, gadoxetic acid, ferric ammonium citrate, mangafodipir, ferumoxsil, ferristene, perflubron, microspheres of human albumin, microparticles of galactose, perflenapent, microspheres of phospholipids, sulfur hexafluoride, and the like.

In one embodiment, the payload is a radiolabel.

As used herein, the terms “radiolabel” or “radiopharmaceutical” refer to any molecule which emits radiation. Radiolabels can be used for therapeutics or diagnostic purposes.

Suitable examples of radiolabels include those described under subgroups V09 and V10 of the Anatomical Therapeutic Chemical Classification System.

Suitable examples of radiolabels include, but are not limited to, ^{99m}Tc compounds (such as, e.g., exametazime, medronic acid, macroaggregated albumin, sestamibi, tetrofosmin, exametazime, sulesomab, tilmanocept, arcitumomab, votumumab, hynic-octreotide, and the like); ¹²³I, ¹²⁵I or ¹³¹I compounds (such as, e.g., ioflupane, iofetamine, iomazenil, sodium iodohippurate, iobenguane, iodocholesterol, minretumomab, tositumomab, and the like); ¹⁸F compounds (such as, e.g., florbetapir, flutemetamol, fluciclovine, fludeoxyglucose, fluoromethyltyrosine, sodium fluoride, and the like); ⁶⁴Cu compounds (such as, e.g., Cu-ETS2, and the like); ⁷⁵Se compounds (such as, e.g., SeHCAT); ¹¹¹In compounds (such as, e.g., imciromab, capromab pendetide, satumomab pendetide, and the like); ⁸²Rb compounds (such as, e.g., rubidium chloride); ¹⁵³Sm compounds (such as, e.g., lexidronam, and the like); ⁸⁹Sr compounds (such as, e.g., strontium-89 chloride, and the like): ⁹⁰Y compounds (such as, e.g., ibritumomab tiuxetan, and the like); ²²³Ra compounds (such as, e.g., radium-223 chloride, and the like); ¹⁷⁷Lu compounds (such as, e.g., oxodotreotide, and the like); and any compounds comprising at least one ²H, ³H, ¹¹C, ¹³N, ¹⁴C, ¹⁵O, ¹⁸F, ²²Na, ²⁴Na, ³²P, ⁴⁷Ca, ⁵¹Cr, ⁵⁷Co, ⁵⁸Co, ⁵⁹Fe, ⁶⁴Cu, ⁶⁷Ga, ⁶⁸Ga, ⁷⁵Se, ⁸¹mKr, ⁸²Rb, ⁸⁹Sr, ⁹⁰Y, ^{99m}Tc, ¹¹¹In, ¹²³I, ¹²⁵I, ¹³¹I, ¹³³Xe, ¹⁵³Sm, ¹⁶⁵Dy, ¹⁶⁹Er, ¹⁷⁷Lu, ¹⁸⁶Re, ¹⁹⁸Au, ²⁰¹Tl and/or ²²³Ra atom.

In one embodiment, the monomer of the STxB protein or of the variant thereof and the payload are bound together through a linker.

A variety of linkers are described in the art and the skilled artisan can readily selected a suitable linker. Linkers may be non-cleavable or cleavable. In the latter, linkers may be protease-sensitive, acid-sensitive, reduction-sensitive or photolabile.

In particular, linkers are preferably selected such that they do not affect the activity of one or both active portions of the conjugate (i.e., the STxB protein or the variant thereof, and/or the payload).

In one embodiment, the linker comprises a polyethylene glycol (PEG). As will be readily understood by the skilled artisan, PEG molecules may be expressed, when located internally within a larger molecule as in the conjugates according to the invention, in the form —(O—CH₂—CH₂)ₙ—, where n is the number of ethylene glycol units. Hence. PEG molecules may be expressed in the form “PEGX”, wherein X is the number of ethylene glycol units.

In one embodiment, the PEG linker can comprise from 2 to 18 ethylene glycol units, i.e., —(O—CH₂—CH₂)ₙ— with 2≤n≤18. The PEG linker can therefore be PEG4, PEG5, PEG6, PEG7, PEG8, PEG9, PEG10, PEG11, PEG12, PEG13, PEG14, PEG15, PEG16, PEG17, or PEG18. In one embodiment, the PEG linker is PEG4.

In one embodiment, the linker comprises a peptide. Examples of peptidic linker are known in art, and include, e.g., glycine-serine linkers.

In one embodiment, the linker can additionally or alternatively comprise a residual portion of a reacted conjugation reagent.

In one embodiment, the STxB monomer conjugate may be obtained by reaction, in suitable conditions, of a payload comprising a chemically reactive moiety, optionally through a linker, with a reactive unnatural amino acid residue of the modified monomer of a STxB protein or of a variant thereof.

Accordingly, the present invention relates to a method of producing a STxB monomer conjugate, as described above, comprising steps of:
a) providing a modified monomer of the STxB protein or of the variant thereof, comprising a substitution with, or an addition of, a reactive unnatural amino acid residue, as described above;
b) providing a payload comprising a chemically reactive moiety, optionally wherein the payload has been previously modified to comprise, optionally through a linker, a chemically reactive moiety;
c) reacting the functional group of the unnatural amino acid residue of said modified monomer of the STxB protein or of the variant thereof with the chemically reactive moiety of the payload, in conditions suitable to form a covalent bound between the modified monomer of the STxB protein or of the variant thereof and the payload, optionally through a linker.

Suitable examples of reactions include, without limitation, an electrophile-nucleophile reaction, an oxime ligation, a ketone reaction with a nucleophile, an aldehyde reaction with a nucleophile, a reaction between a carbonyl group and a nucleophile, a reaction between a sulfonyl group and a nucleophile, an esterification reaction, a reaction between a hindered ester group and a nucleophile, a reaction between a thioester group and a nucleophile, a reaction between a stable imine group and a nucleophile, a reaction between an epoxide group and a nucleophile, a reaction between an aziridine group and a nucleophile, a reaction between an electrophile and an aliphatic or aromatic amine, a reaction between an electrophile and a hydrazide, a reaction between an electrophile and a carbohydrazide, a reaction between an electrophile and a semicarbazide, a reaction between an electrophile and a thiosemicarbazide, a reaction between an electrophile and a carbonylhydrazine, a reaction between an electrophile and a thiocarbonylhydrazide, a reaction between an electrophile and a sulfonylhydrazide, a reaction between an electrophile and a carbazide, a reaction between an electrophile and a thiocarbazide, a reaction between an electrophile and a hydroxylamine, a reaction between a nucleophile or nucleophiles such as a hydroxyl or diol and a boronic acid or ester, a transition metal-catalyzed reaction, a palladium-catalyzed reaction, a copper-catalyzed heteroatom alkylation reaction, a cycloaddition reaction, a 1,3-cycloaddition reaction, a 2,3-cycloaddition reaction, an alkyne-azide reaction, a Diels-Alder reaction, and a Suzuki coupling reaction.

In one embodiment, where the reactive unnatural amino acid residue is selected from azide-functionalized amino acid residues, the STxB monomer conjugate may be obtained by a cycloaddition reaction, in particular by an azide-alkyne cycloaddition reaction. Examples of such azide-alkyne cycloaddition reactions include, but are not limited to, Huisgen azide-alkyne cycloaddition, copper-catalyzed azide-alkyne cycloaddition, ruthenium-catalyzed azide-alkyne cycloaddition, and strain-promoted azide-alkyne cycloaddition reaction.

In particular, strain-promoted azide-alkyne cycloaddition reaction (SPAAC), also named copper-free click reaction, is a bioorthogonal reaction utilizing a pair of reagents, an azide on the one hand and a cyclooctyne on the other hand, that exclusively and efficiently react with each other, forming a stable triazole, while remaining inert to naturally-occurring reactive groups, such as amines Examples of suitable cyclooctynes include dibenzocyclooctynes (DBCO), which is a class of compounds offering fast kinetics, good stability in aqueous buffers, and which does not react with amines or hydroxyls. The examples section herein describes conjugation tests of modified STxB proteins bearing an azide-functionalized amino acid residue with DBCO.

A non-limiting example of method of producing a STxB monomer conjugate is shown in the formula below, wherein “A” is a modified monomer of the STxB protein or of the variant thereof, comprising a reactive unnatural amino acid residue with, as a functional group, an azide moiety (—N═N⁺═N⁻); and “B” is a payload comprising, as chemically reactive moiety, a dibenzocyclooctyne.

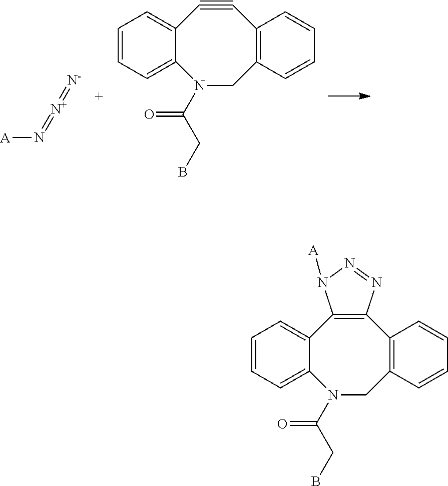

All the reactions recited above are well known to ones skilled in the art, who can readily select the most appropriate reaction according to the reactive unnatural amino acid residue of the modified STxB protein or variant thereof.

The present invention also relates to STxB oligomer conjugates, comprising at least one STxB monomer conjugate as described above.

In one embodiment, the STxB oligomer conjugate is a pentamer comprising or consisting of at least 1, preferably 2, 3, 4 or 5 STxB monomer conjugates, as described above.

In one embodiment, the STxB oligomer conjugate is a homopentamer comprising or consisting of 5 STxB monomer conjugates, as described above, with identical amino acid sequences and identical payloads at the same amino acid positions.

In one embodiment, the STxB oligomer conjugate is a heteropentamer comprising or consisting of 1 STxB monomer conjugate, as described above.

In one embodiment, the STxB oligomer conjugate is a heteropentamer comprising or consisting of 2 STxB monomer conjugates, as described above.

In one embodiment, the STxB oligomer conjugate is a heteropentamer comprising or consisting of 3 STxB monomer conjugates, as described above.

In one embodiment, the STxB oligomer conjugate is a heteropentamer comprising or consisting of 4 STxB monomer conjugates, as described above.

In one embodiment, the STxB oligomer conjugate is a heteropentamer comprising or consisting of 5 STxB monomer conjugates, as described above, wherein at least 2, 3, 4 or 5 of the 5 STxB monomer conjugates have different amino acid sequences and identical payloads.

In one embodiment, the STxB oligomer conjugate is a heteropentamer comprising or consisting of 5 STxB monomer conjugates, as described above, wherein at least 2, 3, 4 or of the 5 STxB monomer conjugates have different amino acid sequences and different payloads.

In one embodiment, the STxB oligomer conjugate is a heteropentamer comprising or consisting of 5 STxB monomer conjugates, as described above, wherein at least 2, 3, 4 or of the 5 STxB monomer conjugates have identical amino acid sequences and different payloads at the same amino acid positions.

In one embodiment, the STxB oligomer conjugate is a heteropentamer comprising or consisting of 5 STxB monomer conjugates, as described above, wherein at least 2, 3, 4 or of the 5 STxB monomer conjugates have identical amino acid sequences and different payloads at different amino acid positions.

In one embodiment, the STxB oligomer conjugate is a heteropentamer comprising or consisting of 5 STxB monomer conjugates, as described above, wherein at least 2, 3, 4 or of the 5 STxB monomer conjugates have identical amino acid sequences and identical payloads at different amino acid positions.

In one embodiment, the STxB oligomer conjugate retains its ability to bind to the glycosphingolipid Gb3/CD77, as described above.

In one embodiment, the STxB oligomer conjugate may be obtained by reaction, in suitable conditions, of a payload comprising a chemically reactive moiety, optionally through a linker, with at least one reactive unnatural amino acid residue of a modified oligomer of a STxB protein or of a variant thereof.

Accordingly, the present invention relates to a method of producing a STxB oligomer conjugate, as described above, comprising steps of:
a) providing a modified oligomer of the STxB protein or of the variant thereof, comprising at least one modified monomer of the STxB protein or of the variant thereof comprising a substitution with, or an addition of, a reactive unnatural amino acid residue, as described above;
b) providing a payload comprising a chemically reactive moiety, optionally wherein the payload has been previously modified to comprise, optionally through a linker, a chemically reactive moiety;
c) reacting the functional group of the unnatural amino acid residue of said at least one modified monomer of the STxB protein or of the variant thereof with the chemically reactive moiety of the payload, in conditions suitable to form a covalent bound between said at least one modified monomer of the STxB protein or of the variant thereof and the payload, optionally through a linker,
   thereby obtaining a STxB oligomer conjugate comprising at least one monomer conjugate.

Suitable examples of reactions have been described above and apply here mutatis mutandis.

The present invention further relates to a composition comprising or consisting of at least one modified monomer of the STxB protein or of the variant thereof as described above.

The present invention further relates to a composition comprising or consisting of at least one modified oligomer of the STxB protein or of the variant thereof as described above.

In one embodiment, the composition comprises or consists of at least one modified pentamer of the STxB protein or of the variant thereof as described above.

The present invention further relates to a composition comprising or consisting of at least one STxB monomer conjugate as described above.

The present invention further relates to a composition comprising or consisting of at least one STxB oligomer conjugate as described above.

In one embodiment, the composition comprises or consists of at least one STxB pentamer conjugate as described above.

In one embodiment, the composition comprises at least 50%, preferably at least 55%, %, 65%, 70%, 80%, 85%, 90%, 95%, 96%, 97%, 98%, 99% or more of the modified monomer of the STxB protein or of the variant thereof, as described above, out of the total monomers of the STxB protein or of the variant thereof in the composition.

In one embodiment, the composition comprises at least 50%, preferably at least 55%, 60%, 65%, 70%, 80%, 85%, 90%, 95%, 96%, 97%, 98%, 99% or more of the modified oligomer of the STxB protein or of the variant thereof, as described above, out of the total oligomers of the STxB protein or of the variant thereof in the composition.

In one embodiment, the composition comprises at least 50%, preferably at least 55%, %, 65%, 70%, 80%, 85%, 90%, 95%, 96%, 97%, 98%, 99% or more of the modified pentamer of the STxB protein or of the variant thereof, as described above, out of the total pentamers of the STxB protein or of the variant thereof in the composition.

In one embodiment, the composition comprises at least 50%, preferably at least 55%, %, 65%, 70%, 80%, 85%, 90%, 95%, 96%, 97%, 98%, 99% or more of the STxB monomer conjugate, as described above, out of the total monomers of the STxB protein or of the variant thereof in the composition.

In one embodiment, the composition comprises at least 50%, preferably at least 55%, %, 65%, 70%, 80%, 85%, 90%, 95%, 96%, 97%, 98%, 99% or more of the STxB oligomer conjugate, as described above, out of the total oligomers of the STxB protein or of the variant thereof in the composition.

In one embodiment, the composition comprises at least 50%, preferably at least 55%, %, 65%, 70%, 80%, 85%, 90%, 95%, 96%, 97%, 98%, 99% or more of the STxB pentamer conjugates, as described above, out of the total pentamers of the STxB protein or of the variant thereof in the composition.

In one embodiment, the composition is a pharmaceutical composition, and further comprises at least one pharmaceutically acceptable excipient.

The term “pharmaceutically acceptable excipient” refers to a solid, semi-solid or liquid component of a pharmaceutical composition or a vaccine composition that is not an active ingredient, and that does not produce an adverse, allergic or other untoward reaction when administered to an animal, preferably to a human. The most of these pharmaceutically acceptable excipients are described in detail in, e.g., Allen (Ed.), 2017. Ansel's pharmaceutical dosage forms and drug delivery systems (11^{th} ed.). Philadelphia, PA: Wolters Kluwer; Remington, Allen & Adeboye (Eds.), 2013. Remington: The science and practice of pharmacy (22^{nd} ed.). London: Pharmaceutical Press; and Sheskey, Cook & Cable (Eds.), 2017. Handbook of pharmaceutical excipients (8^{th} ed.). London: Pharmaceutical Press; each of which is herein incorporated by reference in its entirety.

Pharmaceutically acceptable excipients include, but are not limited to, water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol®, vegetable oils, and the like. One may additionally include suitable preservatives, stabilizers, antioxidants, antimicrobials, and buffering agents, such as, e.g., BHA, BHT, citric acid, ascorbic acid, tetracycline, and the like.

Other examples of pharmaceutically acceptable excipients that may be used in the composition of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In addition, some pharmaceutically acceptable excipients may include, surfactants (e.g., hydroxypropylcellulose); suitable carriers, such as, e.g., solvents and dispersion media containing, e.g., water, ethanol, polyol (e.g., glycerol, propylene glycol, and liquid polyethylene glycol, and the like), suitable mixtures thereof, and vegetable oils, such as, e.g., peanut oil and sesame oil; isotonic agents, such as, e.g., sugars or sodium chloride; coating agents, such as, e.g., lecithin; agents delaying absorption, such as, e.g., aluminum monostearate and gelatin; preservatives, such as, e.g., benzalkonium chloride, benzethonium chloride, chlorobutanol, thimerosal and the like; buffers, such as, e.g., boric acid, sodium and potassium bicarbonate, sodium and potassium borates, sodium and potassium carbonate, sodium acetate, sodium biphosphate and the like; tonicity agents, such as, e.g., dextran 40, dextran 70, dextrose, glycerin, potassium chloride, propylene glycol, sodium chloride; antioxidants and stabilizers, such as, e.g., sodium bisulfite, sodium metabisulfite, sodium thiosulfite, thiourea and the like; nonionic wetting or clarifying agents, such as, e.g., polysorbate 80, polysorbate 20, poloxamer 282 and tyloxapol; viscosity modifying agents, such as, e.g., dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose; and the like.

In one embodiment, the composition is a vaccine composition, and further comprises at least one pharmaceutically acceptable excipient, as described above, and at least one antigen or immunogen.

As used herein, the term “vaccine composition” refers to compositions comprising at least one antigen or immunogen in a pharmaceutically acceptable excipient, and which are useful for inducing an immune response in a subject upon administration.

Examples of antigens or immunogens have been described in details above as suitable payloads of the STxB conjugate. It is however understood that the vaccine composition can comprise at least one antigen or immunogen either in the form of a conjugate to STxB, or in free form (i.e., not conjugated to STxB).

In one embodiment, the vaccine composition further comprises at least one adjuvant.

As used herein, the term “adjuvant” refers to a substance which, when added to a vaccine composition, increases the antigen's or immunogen's immunogenicity (i.e., enhances the immune response to the antigen or immunogen). Thus, an adjuvant is used to modify or increase the effect of a vaccine by stimulating a subject's immune system to respond to the vaccine more vigorously.

Examples of adjuvants include, but are not limited to, aluminum salts (such as, e.g., aluminum hydroxide gel (also named alum), aluminum phosphate, and the like), mineral oil emulsions (such as, e.g., Freund's incomplete adjuvant, Freund's complete adjuvant, and the like), paraffin oil, saponin, Merck adjuvant 65, Smith-Kline Beecham adjuvant AS-2, Aquilla adjuvant QS-21, MPL™ immunostimulant, 3d-MPL, liposomes, lipopolysaccharides (LPS), calcium salts, iron salts (such as, e.g., iron oxide and the like), zinc salts, acylated tyrosine, acylated sugars, cationically-derivatized polysaccharides, anionically-derivatized polysaccharides, glucan, dextran sulfate, sodium alginate, polyphosphazenes, biodegradable microspheres, monophosphoryl lipid A, muramyl tripeptide phosphatidyl ethanolamine, muramyl dipeptide (MDP), cytokines (such as, e.g., interleukin-1, interleukin-2, interleukin-4, interleukin-7, interleukin-12, GM-CSF, TNF-α and the like), helper peptides, components of bacterial cell walls, Corynebacterium parvum, Bacillus Calmette-Guérin, Leishmania eukaryotic initiation factor (LeIF), CpG-containing oligonucleotides (in particular unmethylated CpG-containing oligonucleotides), and combinations thereof.

In one embodiment, the composition is a medicament.

In one embodiment, the composition (including the pharmaceutical composition, the vaccine composition and the medicament) is substantially free of impurities.

As used herein, the term “impurities” broadly refers to any substance other than (1) the STxB protein or a variant thereof, and (2) desired substances (such as pharmaceutically acceptable excipients). Examples of common impurities include, but are not limited to, host cell protein, host cell DNA, cell culture residues (including inducers, antibiotics, serum, media components), downstream processing residues (enzymes, chemical and biochemical processing reagents, inorganic salts, solvents, carriers, ligands), microbial species, endotoxins, pro-inflammatory contaminants, and degradation products.

As used herein, the term “substantially free” with reference to impurities refers to a composition (including the pharmaceutical composition, the vaccine composition and the medicament) which does not include impurities at all or can include them in a residual amount.

In one embodiment, the composition (including the pharmaceutical composition, the vaccine composition and the medicament) comprises less than 20%, preferably less than 19%, 18%, 17%, 16%, 15%, 14%, 13%, 12%, 11%, 10%, 9%, 8%, 7%, 6%, 5%, 4%, 3%, 2%, 1% or less of impurities.

In one embodiment, the composition (including the pharmaceutical composition, the vaccine composition and the medicament) comprises impurities in a concentration that is below a level acceptable to regulatory authorities (including, but not limited to, European Medicines Agency [EMA], Food and Drug Administration [FDA], Pharmaceuticals and Medical Devices Agency [PMDA] and the like) for safe administration to a human or non-human animal.

In one embodiment, the composition (including the pharmaceutical composition, the vaccine composition and the medicament) is substantially free of impurities following guidelines set forth in any of the International Pharmacopoeia 9^{th} edition, the European Pharmacopoeia 10.3, the United States Pharmacopoeia USP 43-NF 38 and/or the Japanese Pharmacopoeia 17^{th} edition.

In one embodiment, the composition (including the pharmaceutical composition, the vaccine composition and the medicament) is substantially free of bacterial endotoxins following guidelines set forth in any of the International Pharmacopoeia 9^{th} edition, the European Pharmacopoeia 10.3, the United States Pharmacopoeia USP 43-NF 38 and/or the Japanese Pharmacopoeia 17^{th} edition.

Bacterial Endotoxins Test (BET) is completely harmonized according to the Q4B annex 14 published by the International Council for Harmonisation in 2012 (International Conference on Harmonisation of Technical Requirements for Registration of Pharmaceuticals for Human Use, 2012. Evaluation and Recommendation of Pharmacopeial Texts for Use in the ICH Regions on Bacterial Endotoxins Test. General Chapter. Q4b Annex 14. Available online at www.ich.org).

In one embodiment, the composition (including the pharmaceutical composition, the vaccine composition and the medicament) is substantially free of bacterial endotoxins as can be assessed according to the guidelines set forth in any of the International Pharmacopoeia 9^{th} edition (section 3.4), the European Pharmacopoeia 10.3 (chapter 5.1.10), the United States Pharmacopoeia USP 43-NF 38 (general chapter <85>) and/or the Japanese Pharmacopoeia 17^{th} edition (section 4.01). The descriptions of apparatuses, reagents, test solutions, preparations, procedures, calculations and interpretations used to detect and/or quantify bacterial endotoxins in these four Pharmacopoeias under their relevant section as described above are herein incorporated by reference in their entirety.

In the International Pharmacopoeia and the United States Pharmacopoeia, three possible alternatives for BET are described: the gel-clot technique, which is based on gel formation; the turbidimetric technique, based on the development of turbidity after cleavage of an endogenous substrate; and the chromogenic technique, based on the development of color after cleavage of a synthetic peptide-chromogen complex.

The Japanese Pharmacopoeia outlines two detailed assays: the gel-clot techniques, which are based on gel formation by the reaction of the lysate TS with endotoxins and the photometric techniques, based on endotoxin-induced optical changes of the lysate TS.

In the European Pharmacopoeia, six methods are described:
method A: gel-clot method limit test;
method B: gel-clot method quantitative test;
method C: turbidimetric kinetic method;
method D: chromogenic kinetic method;
method E: chromogenic end-point method; and
method F: turbidimetric end-point method.

In one embodiment, the composition (including the pharmaceutical composition, the vaccine composition and the medicament) fulfills the requirements for compliance with any of methods A, B, C, D, E, and/or F of the European Pharmacopoeia 10.3 (chapter 5.1.10).

In one embodiment, the composition (including the pharmaceutical composition, the vaccine composition and the medicament) fulfills the requirements for compliance with method A of the European Pharmacopoeia 10.3 (chapter 5.1.10). In one embodiment, the composition (including the pharmaceutical composition, the vaccine composition and the medicament) fulfills the requirements for compliance with method B of the European Pharmacopoeia 10.3 (chapter 5.1.10). In one embodiment, the composition (including the pharmaceutical composition, the vaccine composition and the medicament) fulfills the requirements for compliance with method C of the European Pharmacopoeia 10.3 (chapter 5.1.10). In one embodiment, the composition (including the pharmaceutical composition, the vaccine composition and the medicament) fulfills the requirements for compliance with method D of the European Pharmacopoeia 10.3 (chapter 5.1.10). In one embodiment, the composition (including the pharmaceutical composition, the vaccine composition and the medicament) fulfills the requirements for compliance with method E of the European Pharmacopoeia 10.3 (chapter 5.1.10). In one embodiment, the composition (including the pharmaceutical composition, the vaccine composition and the medicament) fulfills the requirements for compliance with method F of the European Pharmacopoeia 10.3 (chapter 5.1.10).

In one embodiment, the composition (including the pharmaceutical composition, the vaccine composition and the medicament) comprises less than 50 endotoxin units (EU) per mg of STxB protein or of the variant thereof, preferably less than 45, 40, 35, 30, 25, 15, 10, 9, 8, 7, 6, 5, 4, 3, 2, 1 or less EU/mg of STxB protein or of the variant thereof.

In one embodiment, the composition (including the pharmaceutical composition, the vaccine composition and the medicament) comprises endotoxins in an amount ranging from about 50 to 0 EU/mg of STxB protein or of a variant thereof, preferably from about 40 to 0, from about 30 to 0, from about 20 to 0, from about 15 to 0, from about 10 to 0, or from about 5 to 0 EU/mg of STxB protein or of a variant thereof.

In one embodiment, the composition (including the pharmaceutical composition, the vaccine composition and the medicament) is formulated for administration to a subject.

In one embodiment, the composition (including the pharmaceutical composition, the vaccine composition and the medicament) is formulated for systemic or local administration to a subject.

In one embodiment, the composition (including the pharmaceutical composition, the vaccine composition and the medicament) is formulated for administration by injection, oral administration, topical administration, nasal administration, buccal administration, rectal administration, vaginal administration, intratracheal administration, administration by endoscopy, transmucosal administration, percutaneous administration, or intratumoral administration.

In one embodiment, the composition (including the pharmaceutical composition, the vaccine composition and the medicament) is formulated for administration by injection, preferably by systemic injection.

Examples of formulations adapted for injection include, but are not limited to, solutions, such as, e.g., sterile aqueous solutions, gels, dispersions, emulsions, suspensions, solid forms suitable for using to prepare solutions or suspensions upon the addition of a liquid prior to use, such as, for example, powder, liposomal forms and the like.

Examples of systemic injections include, but are not limited to, intravenous (iv), subcutaneous (sq), intradermal (id), intramuscular (im), intraarterial, intraparenteral, intranodal, intralymphatic, intraperitoneal (ip), intracranial, intracardiac, intralesional, intraprostatic, intravaginal, intrarectal, intrathecal, intranasal, intratumoral (it), intravesicular, and perfusion.

In one embodiment, when injected, the composition (including the pharmaceutical composition, the vaccine composition and the medicament) is sterile. Methods for obtaining a sterile composition include, but are not limited to, GMP synthesis (where GMP stands for “Good manufacturing practice”).

Sterile injectable forms of a composition may be aqueous or oleaginous. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

It will be understood that other suitable routes of administration are also contemplated in the present invention, and the administration mode will ultimately be decided by the attending physician within the scope of sound medical judgment.

In one embodiment, the composition (including the pharmaceutical composition, the vaccine composition and the medicament) is to be administered to a subject in need thereof before, concomitantly with, or after administration of at least one additional therapeutic or diagnostic agent.

Examples of additional therapeutic or diagnostic agents include all those described in details above as suitable payloads of the STxB conjugate, including, but not limited to, chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, antigens, hormones, coding or non-coding oligonucleotides, photodetectable labels, contrast agents, radiolabels, and the like. It is however understood that the composition (including the pharmaceutical composition, the vaccine composition and the medicament) can comprise the at least one additional therapeutic or diagnostic agent either in the form of a STxB monomer or oligomer conjugate, as described above; or in free form (i.e., not conjugated to STxB); or both.

Consistently, the present invention also relates to a composition (including a pharmaceutical composition, a vaccine composition and a medicament) comprising or consisting of at least one modified monomer of the STxB protein or of the variant thereof as described above; and at least one additional therapeutic or diagnostic agent as described above.

Consistently, the present invention also relates to a composition (including a pharmaceutical composition, a vaccine composition and a medicament) comprising or consisting of at least one modified oligomer of the STxB protein or of the variant thereof, as described above; and at least one additional therapeutic or diagnostic agent as described above.

Consistently, the present invention also relates to a composition (including a pharmaceutical composition, a vaccine composition and a medicament) comprising or consisting of at least one STxB monomer conjugate as described above; and at least one additional therapeutic or diagnostic agent as described above.

Consistently, the present invention also relates to a composition (including a pharmaceutical composition, a vaccine composition and a medicament) comprising or consisting of at least one STxB oligomer conjugate as described above; and at least one additional therapeutic or diagnostic agent as described above.

In one embodiment, the composition (including the pharmaceutical composition, the vaccine composition and the medicament), optionally further comprising at least one additional therapeutic or diagnostic agent, is to be administered to a subject in need thereof before, concomitantly with, or after at least one regimen of radiation therapy, such as 1, 2, 3, 4, 5, 6, 7, 8, 9, 10, 11, 12, 13, 14, 15, 20, 25, 30, or more than 30 regimens of radiation therapy.

Suitable examples of radiation therapies include, but are not limited to, external beam radiotherapy (such as, e.g., superficial X-rays therapy, orthovoltage X-rays therapy, megavoltage X-rays therapy, radiosurgery, stereotactic radiation therapy, cobalt therapy, electron therapy, fast neutron therapy, neutron-capture therapy, proton therapy, and the like); brachytherapy; unsealed source radiotherapy; tomotherapy; and the like.

As will be further detailed hereafter, the modified monomer or the modified oligomer of the STxB protein or of the variant thereof, the STxB monomer or oligomer conjugate, as well as the composition (including the pharmaceutical composition, the vaccine composition and the medicament), optionally further comprising at least one additional therapeutic or diagnostic agent, are useful for a wide range of therapeutic and diagnostic purposes. For a review, see Johannes & Römer, 2010. Nat Rev Microbiol. 8(2):105-16; Engedal et al., 2011. Microb Biotechnol. 4(1):32-46; Adkins et al., 2012. Curr Pharm Biotechnol. 13(8):1446-73; Bergan et al., 2012. Toxicon. 60(6):1085-107; Lee et al., 2016. Toxins (Basel). 8(3); Luginbuehl et al., 2018. Biotechnol Adv. 36(3):613-623; the content of each of these being herein incorporated by reference in its entirety.

The present invention further relates to a method of treating a disease in a subject in need thereof, comprising or consisting of administering to said subject the modified monomer or the modified oligomer of the STxB protein or of the variant thereof, the STxB monomer or oligomer conjugate, or the composition (including the pharmaceutical composition, the vaccine composition and the medicament).

Alternatively, the present invention relates to the modified monomer or the modified oligomer of the STxB protein or of the variant thereof, to the STxB monomer or oligomer conjugate, or to the composition (including the pharmaceutical composition, the vaccine composition and the medicament), for use in a method of treating a disease in a subject in need thereof.

In one embodiment, the method of treating a disease in a subject in need thereof further comprises administering to said subject at least one additional therapeutic or diagnostic agent as described above. In one embodiment, the at least one additional therapeutic or diagnostic agent is to be administered before, concomitantly with, or after administration of the modified monomer or the modified oligomer of the STxB protein or of the variant thereof, the STxB monomer or oligomer conjugate, or the composition (including the pharmaceutical composition, the vaccine composition and the medicament).

In one embodiment, the method of treating a disease in a subject in need thereof further comprises administering to said subject at least one regimen of radiation therapy as described above. In one embodiment, the at least one regimen of radiation therapy is to be administered before, concomitantly with, or after administration of the modified monomer or the modified oligomer of the STxB protein or of the variant thereof, the STxB monomer or oligomer conjugate, or the composition (including the pharmaceutical composition, the vaccine composition and the medicament).

In one embodiment, the method of treating a disease in a subject in need thereof further comprises administering to said subject at least one additional therapeutic or diagnostic agent as described above; and at least one regimen of radiation therapy as described above. In one embodiment, the at least one additional therapeutic or diagnostic agent and the at least one regimen of radiation therapy are each to be administered before, concomitantly with, or after administration of the modified monomer or the modified oligomer of the STxB protein or of the variant thereof, the STxB monomer or oligomer conjugate, or the composition (including the pharmaceutical composition, the vaccine composition and the medicament).

In one embodiment, the disease is cancer, an infectious disease, an immune disorder and/or an inflammatory disorder.

In one embodiment, the disease is cancer.

Examples of cancers include those listed in the 10^{th} revision of the International Statistical Classification of Diseases and Related Health Problems (ICD), under chapter II, blocks COO to D48.

Further examples of cancers include, but are not limited to, recurrent, metastatic or multi-drug resistant cancer.

Further examples of cancers include, but are not limited to, adenofibroma, adenoma, agnogenic myeloid metaplasia, AIDS-related malignancies, ameloblastoma, anal cancer, angiofollicular mediastinal lymph node hyperplasia, angiokeratoma, angiolymphoid hyperplasia with eosinophilia, angiomatosis, anhidrotic ectodermal dysplasia, anterofacial dysplasia, apocrine metaplasia, apudoma, asphyxiating thoracic dysplasia, astrocytoma (including, e.g., cerebellar astrocytoma and cerebral astrocytoma), atriodigital dysplasia, atypical melanocytic hyperplasia, atypical metaplasia, autoparenchymatous metaplasia, basal cell hyperplasia, benign giant lymph node hyperplasia, bile duct cancer (including, e.g., extrahepatic bile duct cancer), bladder cancer, bone cancer, brain tumor (including, e.g., brain stem glioma, cerebellar astrocytoma glioma, malignant glioma, supratentorial primitive neuroectodermal tumors, visual pathway and hypothalamic glioma, ependymoma, medulloblastoma, gestational trophoblastic tumor glioma, and paraganglioma), branchionia, female breast cancer, male breast cancer, bronchial adenomas/carcinoids, bronchopulmonary dysplasia, cancer growths of epithelial cells, pre-cancerous growths of epithelial cells, metastatic growths of epithelial cells, carcinoid heart disease, carcinoid tumor (including, e.g., gastrointestinal carcinoid tumor), carcinoma (including, e.g., carcinoma of unknown primary origin, adrenocortical carcinoma, islet cells carcinoma, adeno carcinoma, adeoncortical carcinoma, basal cell carcinoma, basosquamous carcinoma, bronchiolar carcinoma, Brown-Pearce carcinoma, cystadenocarcinoma, ductal carcinoma, hepatocarcinoma, Krebs carcinoma, papillary carcinoma, oat cell carcinoma, small cell lung carcinoma, non-small cell lung carcinoma, squamous cell carcinoma, transitional cell carcinoma, Walker carcinoma, Merkel cell carcinoma, and skin carcinoma), cementoma, cementum hyperplasia, cerebral dysplasia, cervical cancer, cervical dysplasia, cholangioma, cholesteatoma, chondroblastoma, chondroectodermal dysplasia, chordoma, choristoma, chrondroma, cleidocranial dysplasia, colon cancer, colorectal cancer, local metastasized colorectal cancer, congenital adrenal hyperplasia, congenital ectodermal dysplasia, congenital sebaceous hyperplasia, connective tissue metaplasia, craniocarpotarsal dysplasia, craniodiaphysial dysplasia, craniometaphysial dysplasia, craniopharyngioma, cylindroma, cystadenoma, cystic hyperplasia (including, e.g., cystic hyperplasia of the breast), cystosarconia phyllodes, dentin dysplasia, denture hyperplasia, diaphysial dysplasia, ductal hyperplasia, dysgenninoma, dysplasia epiphysialis hemimelia, dysplasia epiphysialis multiplex, dysplasia epiphysialis punctate, ectodermal dysplasia, Ehrlich tumor, enamel dysplasia, encephaloophthalmic dysplasia, endometrial cancer (including, e.g., ependymoma and endometrial hyperplasia), ependymoma, epithelial cancer, epithelial dysplasia, epithelial metaplasia, esophageal cancer, Ewing's family of tumors (including, e.g., Ewing's sarcoma), extrahepatic bile duct cancer, eye cancer (including, e.g., intraocular melanoma and retinoblastoma), faciodigitogenital dysplasia, familial fibrous dysplasia of jaws, familial white folded dysplasia, fibroma, fibromuscular dysplasia, fibromuscular hyperplasia, fibrous dysplasia of bone, florid osseous dysplasia, focal epithelial hyperplasia, gall bladder cancer, ganglioneuroma, gastric cancer (including, e.g., stomach cancer), gastrointestinal carcinoid tumor, gastrointestinal tract cancer, gastrointestinal tumors, Gaucher's disease, germ cell tumors (including, e.g., extracranial germ cell tumors, extragonadal germ cell tumors, and ovarian germ cell tumors), giant cell tumor, gingival hyperplasia, glioblastoma, glomangioma, granulosa cell tumor, gynandroblastoma, hamartoma, head and neck cancer, hemangioendothelioma, hemangioma, hemangiopericytoma, hepatocellular cancer, hepatoma, hereditary renal-retinal dysplasia, hidrotic ectodermal dysplasia, histiocytonia, histiocytosis, hypergammaglobulinemia, hypohidrotic ectodermal dysplasia, hypopharyngeal cancer, inflammatory fibrous hyperplasia, inflammatory papillary hyperplasia, intestinal cancers, intestinal metaplasia, intestinal polyps, intraocular melanoma, intravascular papillary endothelial hyperplasia, kidney cancer, laryngeal cancer, leiomyoma, leukemia (including, e.g., acute lymphoblastic leukemia, acute lymphocytic leukemia, acute myeloid leukemia, acute myelogenous leukemia, acute hairy cell leukemia, acute B-cell leukemia, acute T-cell leukemia, acute HTLV leukemia, chronic lymphoblastic leukemia, chronic lymphocytic leukemia, chronic myeloid leukemia, chronic myelogenous leukemia, chronic hairy cell leukemia, chronic B-cell leukemia, chronic T-cell leukemia, and chronic HTLV leukemia), Leydig cell tumor, lip and oral cavity cancer, lipoma, liver cancer, lung cancer (including, e.g., small cell lung cancer and non-small cell lung cancer), lymphangiomyoma, lymphaugioma, lymphoma (including, e.g., AIDS-related lymphoma, central nervous system lymphoma, primary central nervous system lymphoma, Hodgkin's lymphoma, non-Hodgkin's lymphoma, Hodgkin's lymphoma during pregnancy, non-Hodgkin's lymphoma during pregnancy, mast cell lymphoma, B-cell lymphoma, adenolymphoma, Burkitt's lymphoma, cutaneous T-cell lymphoma, large cell lymphoma, and small cell lymphoma), lymphopenic thymic dysplasia, lymphoproliferative disorders, macroglobulinemia (including, e.g., Waldenstrom's macroglobulinemia), malignant carcinoid syndrome, malignant mesothelioma, malignant thymoma, mammary dysplasia, mandibulofacial dysplasia, medulloblastoma, meningioma, mesenchymoma, mesonephroma, mesothelioma (including, e.g., malignant mesothelioma), metaphysial dysplasia, metaplastic anemia, metaplastic ossification, metaplastic polyps, metastatic squamous neck cancer (including, e.g., metastatic squamous neck cancer with occult primary), Mondini dysplasia, monostotic fibrous dysplasia, mucoepithelial dysplasia, multiple endocrine neoplasia syndrome, multiple epiphysial dysplasia, multiple myeloma/plasma cell neoplasm, mycosis fungoides, myelodysplastic syndrome, myeloid metaplasia, myeloproliferative disorders, chronic myeloproliferative disorders, myoblastoma, myoma, myxoma, nasal cavity and paranasal sinus cancer, nasopharyngeal cancer, prostatic neoplasm, colon neoplasm, abdomen neoplasm, bone neoplasm, breast neoplasm, digestive system neoplasm, liver neoplasm, pancreas neoplasm, peritoneum neoplasm, endocrine glands neoplasm (including, e.g., adrenal neoplasm, parathyroid neoplasm, pituitary neoplasm, testicles neoplasm, ovary neoplasm, thymus neoplasm, and thyroid neoplasm), eye neoplasm, head and neck neoplasm, nervous system neoplasm (including, e.g., central nervous system neoplasm and peripheral nervous system neoplasm), lymphatic system neoplasm, pelvic neoplasm, skin neoplasm, soft tissue neoplasm, spleen neoplasm, thoracic neoplasm, urogenital tract neoplasm, neurilemmoma, neuroblastoma, neuroepithelioma, neurofibroma, neurofibromatosis, neuroma, nodular hyperplasia of prostate, nodular regenerative hyperplasia, oculoauriculovertebral dysplasia, oculodentodigital dysplasia, oculovertebral dysplasia, odontogenic dysplasia, odontoma, opthalmomandibulomelic dysplasia, oropharyngeal cancer, osteoma, ovarian cancer (including, e.g., ovarian epithelial cancer and ovarian low malignant potential tumor), pancreatic cancer (including, e.g., islet cell pancreatic cancer and exocrine pancreatic cancer), papilloma, paraganglioma, nonchromaffin paraganglioma, paranasal sinus and nasal cavity cancer, paraproteinemias, parathyroid cancer, periapical cemental dysplasia, pheochromocytoma (including, e.g., penile cancer), pineal and supratentorial primitive neuroectodermal tumors, pinealoma, pituitary tumor, plasma cell neoplasm/multiple myeloma, plasmacytoma, pleuropulmonary blastoma, polyostotic fibrous dysplasia, polyps, pregnancy cancer, pre-neoplastic disorders (including, e.g., benign dysproliferative disorders such as benign tumors, fibrocystic conditions, tissue hypertrophy, intestinal polyps, colon polyps, esophageal dysplasia, leukoplakia, keratoses, Bowen's disease, Farmer's skin, solar cheilitis, and solar keratosis), primary hepatocellular cancer, primary liver cancer, primary myeloid metaplasia, prostate cancer, pseudoachondroplastic spondyloepiphysial dysplasia, pseudoepitheliomatous hyperplasia, purpura, rectal cancer, renal cancer (including, e.g., kidney cancer, renal pelvis, ureter cancer, transitional cell cancer of the renal pelvis and ureter), reticuloendotheliosis, retinal dysplasia, retinoblastoma, salivary gland cancer, sarcomas (including, e.g., uterine sarcoma, soft tissue sarcoma, carcinosarcoma, chondrosarcoma, fibrosarcoma, hemangiosarcoma, Kaposi's sarcoma, leiomyosarcoma, liposarcoma, lymphangiosarcoma, myosarcoma, myxosarcoma, rhabdosarcoma, sarcoidosis sarcoma, osteosarcoma, Ewing sarcoma, malignant fibrous histiocytoma of bone, and clear cell sarcoma of tendon sheaths), sclerosing angioma, secondary myeloid metaplasia, senile sebaceous hyperplasia, septooptic dysplasia, Sertoli cell tumor, Sezary syndrome, skin cancer (including, e.g., melanoma skin cancer and non-melanoma skin cancer), small intestine cancer, spondyloepiphysial dysplasia, squamous metaplasia (including, e.g., squamous metaplasia of amnion), stomach cancer, supratentorial primitive neuroectodermal and pineal tumors, supratentorial primitive neuroectodermal tumors, symptomatic myeloid metaplasia, teratoma, testicular cancer, theca cell tumor, thymoma (including, e.g., malignant thymoma), thyroid cancer, trophoblastic tumors (including, e.g., gestational trophoblastic tumors), ureter cancer, urethral cancer, uterine cancer, vaginal cancer, ventriculoradial dysplasia, verrucous hyperplasia, vulvar cancer, Waldenstrom's macroglobulinemia, and Wilms' tumor.

In one embodiment, the disease is an infectious disease.

Examples of infectious diseases include those listed in the 10^{th} revision of the International Statistical Classification of Diseases and Related Health Problems (ICD), under chapter I, blocks A00 to B99.

Further examples of infectious diseases include, but are not limited to, bacterial infections, viral infections, fungal infections, parasitic infections, ectoparasitic infections, and the like.

In one embodiment, the disease is an immune disorder.

Examples of immune disorders include those listed in the 10^{th} revision of the International Statistical Classification of Diseases and Related Health Problems (ICD), under chapter III, blocks D80 to D89.

Further examples of immune disorders include, but are not limited to, lymphoid immunodeficiencies, complement immunodeficiencies, monocyte immunodeficiencies, granulocyte immunodeficiencies, and phagocyte bactericidal dysfunctions.

Further examples of immune disorders include, but are not limited to, hypogammaglobulinemia (such as, e.g., X-linked agammaglobulinemia, transient hypogammaglobulinemia of infancy, and the like); dysgammaglobulinemia (such as, e.g., IgA deficiency, IgG deficiency, IgM deficiency, hyper IgM syndrome type 1, hyper IgM syndrome type 2, hyper IgM syndrome type 3, hyper IgM syndrome type 4, hyper IgM syndrome type 5, Wiskott-Aldrich syndrome, hyper-IgE syndrome, and the like); common variable immunodeficiency; ICF syndrome; thymic hypoplasia (such as, e.g., Di George's syndrome, Nezelof syndrome, ataxia-telangiectasia, and the like); purine nucleoside phosphorylase deficiency; X-linked severe combined immunodeficiency; adenosine deaminase deficiency; Omenn syndrome; ZAP70 deficiency; Bare lymphocyte syndrome; lymphocytopenia (such as, e.g., T lymphocytopenia, B lymphocytopenia, NK lymphocytopenia, and the like); complement deficiency (such as, e.g., angioedema, hereditary angioedema, complement 2 deficiency/complement 4 deficiency, MBL deficiency, properdin deficiency, complement 3 deficiency, terminal complement pathway deficiency, paroxysmal nocturnal hemoglobinuria, complement receptor deficiency, and the like); histiocytosis; chronic granulomatous disease; monocytosis; monocytopenia; granulocytosis (such as, e.g., neutrophilia, eosinophilia, hypereosinophilic syndrome, basophilia, bandemia, and the like); granulocytopenia and agranulocytosis (such as, e.g., neutropenia, Kostmann syndrome, eosinopenia, basopenia, and the like); phagocyte bactericidal dysfunctions (such as, e.g., leukocyte adhesion deficiency-1, leukocyte adhesion deficiency-2, Chédiak-Higashi syndrome, neutrophil-specific granule deficiency, chronic granulomatous disease, neutrophil immunodeficiency syndrome, myeloperoxidase deficiency, and the like).

In one embodiment, the disease is an inflammatory disorder.

Examples of inflammatory disorders include, but are not limited to, abdominal aortic aneurysm (AAA), acne, acute disseminated encephalomyelitis, acute leukocyte-mediated lung injury, Addison's disease, adult respiratory distress syndrome, AIDS dementia, allergic asthma, allergic conjunctivitis, allergic rhinitis, allergic sinusitis, alopecia areata, Alzheimer's disease, anaphylaxis, angioedema, ankylosing spondylitis, antiphospholipid antibody syndrome, asthma, atopic dermatitis, autoimmune hemolytic anemia, autoimmune hepatitis, autoimmune inner ear disease, Behcet's syndrome, blepharitis, bronchitis, bullous pemphigoid, Chagas' disease, chronic inflammatory diseases, chronic obstructive pulmonary disease, coagulative necrosis, coeliac disease, collagenous colitis, conjunctivitis, contact dermatitis, coronary heart disease, cutaneous necrotizing venulitis, cystic fibrosis, dermatitis, dermatomyositis, diabetes mellitus type 1, diabetes mellitus type 2, distal proctitis, diversion colitis, dry eye, eczema, encephalitis, endometriosis, endotoxin shock, epilepsy, erythema multiforme, erythema nodosum, fibrinoid necrosis, fibromyalgia, giant-cell arteritis (Horton's disease), goodpasture's syndrome, gouty arthritis, graft-versus-host disease (such as, e.g., acute graft-versus-host disease, chronic graft-versus-host disease, and the like), Graves' disease, Guillain-Barre syndrome, Hashimoto's disease, hay fever, hyperacute transplant rejection, hyperlipidemia, idiopathic thrombocytopenic purpura, indeterminate colitis, infective colitis, inflammatory bowel disease (IBD) (such as, e.g., Crohn's disease, ulcerative colitis, colitis, and the like), inflammatory liver disorder, insect bite skin inflammation, interstitial cystitis, iritis, ischaemic colitis, lichen planus, liquefactive necrosis, lupus erythematosus, lymphocytic colitis, meningitis, metabolic syndrome, multiple sclerosis, myasthenia gravis, myocarditis, narcolepsy, nephritis, obesity, pancreatitis, Parkinson's disease, pemphigus vulgaris, periodontal gingivitis, periodontitis, pernicious anaemia, polymyalgia rheumatica, polymyositis, postmenopausal-induced metabolic syndrome, primary biliary cirrhosis, psoriasis, retinitis, rheumatoid arthritis, rheumatoid spondylitis, rhinoconjunctivitis, scleroderma, shingles, Sjogren's syndrome, smooth muscle proliferation disorders, solar dermatitis, steatosis, systemic lupus erythematosus (SLE), tuberculosis, urticaria, uveitis, vasculitis, vitiligo, and Wegener's granulomatosis.

In one embodiment, the STxB monomer or oligomer conjugate comprises a payload, and/or the at least one additional therapeutic or diagnostic agent is a payload, said payload being selected from the group comprising or consisting of chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, antigens, hormones, and coding or non-coding oligonucleotides, as described above.

The present invention further relates to a method of vaccinating a subject in need thereof, comprising or consisting of administering to said subject the modified monomer or the modified oligomer of the STxB protein or of the variant thereof, the STxB monomer or oligomer conjugate, or the composition (including the pharmaceutical composition, the vaccine composition and the medicament).

Alternatively, the present invention relates to the modified monomer or the modified oligomer of the STxB protein or of the variant thereof, to the STxB monomer or oligomer conjugate, or to the composition (including the pharmaceutical composition, the vaccine composition and the medicament), for use in a method of vaccinating in a subject in need thereof.

In one embodiment, the method of vaccinating in a subject in need thereof further comprises administering to said subject at least one additional therapeutic or diagnostic agent as described above. In one embodiment, the at least one additional therapeutic or diagnostic agent is to be administered before, concomitantly with, or after administration of the modified monomer or the modified oligomer of the STxB protein or of the variant thereof, the STxB monomer or oligomer conjugate, or the composition (including the pharmaceutical composition, the vaccine composition and the medicament).

In one embodiment, the STxB monomer or oligomer conjugate comprises a payload, and/or the at least one additional therapeutic or diagnostic agent is a payload, said payload being selected from the group comprising or consisting of antigens or immunogens, as described above.

The present invention further relates to a method of combinatorial immunotherapy in a subject in need thereof, comprising or consisting of administering to said subject the modified monomer or the modified oligomer of the STxB protein or of the variant thereof, the STxB monomer or oligomer conjugate, or the composition (including the pharmaceutical composition, the vaccine composition and the medicament).

Alternatively, the present invention relates to the modified monomer or the modified oligomer of the STxB protein or of the variant thereof, to the STxB monomer or oligomer conjugate, or to the composition (including the pharmaceutical composition, the vaccine composition and the medicament), for use in a method of combinatorial immunotherapy in a subject in need thereof.

Such combination immunotherapies are well known to the one skilled in the art. See, e.g., Swart et al., 2016. Front Oncol. 6:233; and Collins et al., 2018. Expert Rev Vaccines. 17(8):697-705.

In one embodiment, the STxB monomer or oligomer conjugate comprises a payload, and/or the at least one additional therapeutic or diagnostic agent is a payload, said payload being selected from the group comprising or consisting of chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, antigens, hormones, and coding or non-coding oligonucleotides, as described above. In one embodiment, the conjugate comprises two payloads for combination immunotherapy.

The present invention further relates to the use of the modified monomer or the modified oligomer of the STxB protein or of the variant thereof, of the STxB monomer or oligomer conjugate, or of the composition (including the pharmaceutical composition, the vaccine composition and the medicament), as a contrast agent in a method of medical imaging of a subject in need thereof.

Alternatively, the present invention relates to the modified monomer or the modified oligomer of the STxB protein or of the variant thereof, to the STxB monomer or oligomer conjugate, or to the composition (including the pharmaceutical composition, the vaccine composition and the medicament), for use as a contrast agent in a method of medical imaging of a subject in need thereof.

In one embodiment, the use as a contrast agent further comprises administering at least one additional therapeutic or diagnostic agent as described above. In one embodiment, the at least one additional therapeutic or diagnostic agent is to be administered before, concomitantly with, or after administration of the modified monomer or the modified oligomer of the STxB protein or of the variant thereof, the STxB monomer or oligomer conjugate, or the composition (including the pharmaceutical composition, the vaccine composition and the medicament).

In one embodiment, the use as a contrast agent further comprises administering at least one regimen of radiation therapy as described above. In one embodiment, the at least one regimen of radiation therapy is to be administered before, concomitantly with, or after administration of the modified monomer or the modified oligomer of the STxB protein or of the variant thereof, the STxB monomer or oligomer conjugate, or the composition (including the pharmaceutical composition, the vaccine composition and the medicament).

In one embodiment, the use as a contrast agent further comprises administering at least one additional therapeutic or diagnostic agent as described above; and at least one regimen of radiation therapy as described above. In one embodiment, the at least one additional therapeutic or diagnostic agent and the at least one regimen of radiation therapy are each to be administered before, concomitantly with, or after administration of the modified monomer or the modified oligomer of the STxB protein or of the variant thereof, the STxB monomer or oligomer conjugate, or the composition (including the pharmaceutical composition, the vaccine composition and the medicament).

In one embodiment, the STxB monomer or oligomer conjugate comprises a payload, and/or the at least one additional therapeutic or diagnostic agent is a payload, said payload being selected from the group comprising or consisting of photodetectable labels, contrast agents and radiolabels, as described above.

In one embodiment, the use as a contrast agent allows to detect Gb3-expressing cells in a subject. In particular, the use as a contrast agent allows to detect Gb3-expressing tumor cells in a subject.

The present invention further relates to a method of diagnosing a disease in a subject in need thereof, comprising or consisting of administering to said subject the modified monomer or the modified oligomer of the STxB protein or of the variant thereof, the STxB monomer or oligomer conjugate, or the composition (including the pharmaceutical composition, the vaccine composition and the medicament).

Alternatively, the present invention relates to the modified monomer or the modified oligomer of the STxB protein or of the variant thereof, to the STxB monomer or oligomer conjugate, or to the composition (including the pharmaceutical composition, the vaccine composition and the medicament), for use in an in vivo method of diagnosis of a disease in a subject in need thereof.

In one embodiment, the disease is cancer, an infectious disease and/or an immune disorder.

Suitable examples of cancer, an infectious disease and/or an immune disorder have been described above.

As used herein, the term “diagnosis” broadly refers to the diagnosis per se, i.e., the identification of a disease by observation of signs and/or symptoms; but also includes the prognosis and recurrence monitoring of the disease. The term “prognosis” refers to a prediction of the course and outcomes of a disease, including whether the signs and symptoms will improve or worsen (and how quickly) or remain stable over time; expectations of quality of life, such as the ability to carry out daily activities; the potential for complications and associated health issues; and the likelihood of survival (including life expectancy). The term “prognosis” also encompasses the prediction of the course and outcomes of the disease during a therapy, and the assessment of the efficiency of a therapy to treat the given disease. The term “recurrence” refers to the reappearance of a disease.

In one embodiment, the STxB monomer or oligomer conjugate comprises a payload, and/or the at least one additional therapeutic or diagnostic agent is a payload, said payload being selected from the group comprising or consisting of photodetectable labels, contrast agents and radiolabels, as described above.

### BRIEF DESCRIPTION OF THE DRAWINGS

FIG. 1 is a set of immunofluorescence microscopy photographs of an intracellular trafficking assay on HeLa cells incubated with 0.2 μM (monomer concentration) of STxB variants their corresponding conjugates with the N-terminally extended version of the OVA₂₅₇₋₂₆₄ peptide (SL8 peptide). [rSTxB]: recombinant STxB with SEQ ID NO: 22; [sSTxB]: synthetic STxB-Cter-70-A-N₃—CONH₂ with SEQ ID NO: 24; [JU57]: rSTxB/bromoacetyl-SL8 conjugate; [ABILC2]: sSTxB/DBCO-PEG4-SL8 conjugate. Merge: STxB channel in green, giantin (a Golgi membrane protein) channel in magenta, and DNA dye Hoechst channel in blue. FIG. 1 is executed in color.

FIG. 2 is a histogram showing a comparative analysis of the ability of an N-terminally extended version of the OVA₂₅₇₋₂₆₄ peptide, either in free form [SL8 peptide] or coupled to recombinant [JU57] or synthetic [ABILC2] STxB to elicit specific anti-OVA specific CD8⁺T cells. The histogram represents the percentage of H2 K^{b} OVA₂₅₇₋₂₆₄ tetramer among total CD8⁺ T cells in broncho-alveolar lavages (BAL), for each vaccine.

FIGS. 3A-C are a set of flow plots and histograms showing a comparative analysis of the ability of an N-terminally extended version of the OVA₂₅₇₋₂₆₄ peptide, either in free form [SL8 peptide] or coupled to recombinant [JU57] or synthetic [ABILC2] STxB to elicit resident memory T cells (T_{RM}). FIG. 3A shows the proportions of CD103/CD49a cells in lungs upon immunization with an N-terminally extended version of the OVA₂₅₇₋₂₆₄ peptide, either in free form [SL8 peptide] or coupled to recombinant [JU57] or synthetic [ABILC2] STxB. T cells expressing CD103 and/or CD49a correspond to T_{RM}; CD103⁻/CD49a⁻ cells are effector T cells (T_{eff} cells). FIG. 3B shows the absolute number of T_{RM} anti-OVA CD8⁺ T cells in broncho-alveolar lavages (BAL), for each vaccine. FIG. 3C shows the absolute number of T_{RM} anti-OVA CD8⁺ T cells in the lung parenchyma.

### EXAMPLES

The present invention is further illustrated by the following examples, but is not to be construed as being limited thereto.

### Example 1

Materials and Methods

A screen of STxB with SEQ ID NO: 2, for incorporation of azide-functionalized amino acid residues, was performed, in order to identify positions that are permissive for unnatural amino acid incorporation, and which allow efficient site-specific conjugation without affecting the stability or trafficking characteristics of STxB.

STxB Variants Choice

Using Pymol software, potential positions for azide-functionalized amino acid residues incorporation were selected based on the analysis of the STxB structure in complex with an analog of its receptor (protein data bank file: 1BOS; Ling et al., 1998. Biochemistry. 37(7):1777-88).

Key criteria for these potential positions were:
i) the amino acid residue should not be exposed in Gb3 binding sites, and
ii) the amino acid residue should be exposed on the surface of the STxB pentamer.

A Met48Nle substitution was also tested to replace the methionine at position 48 (SEQ ID NO: 2 numbering) which is prone to oxidation.

Unnatural Amino Acids Used

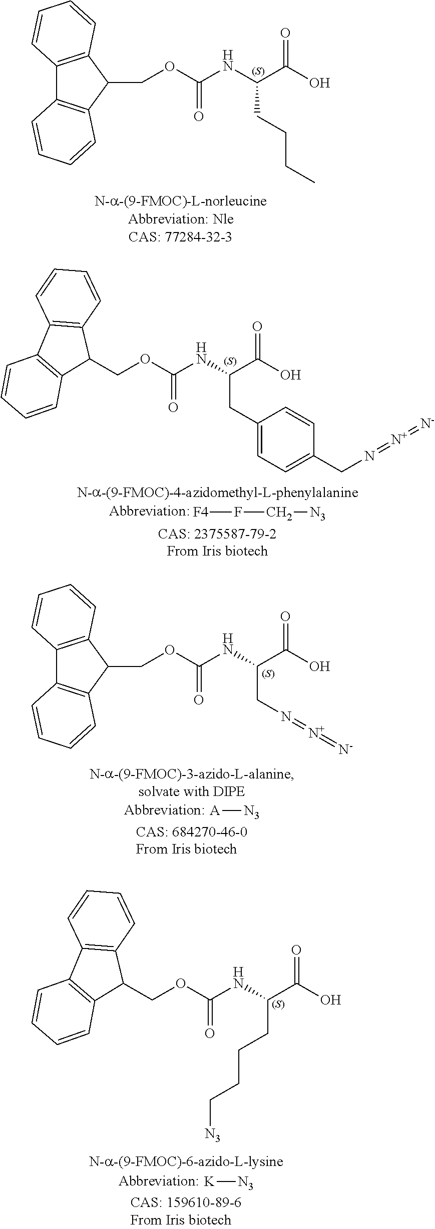

FMOC: fluorenylmethyloxycarbonyl

Selected Monomeric STxB Variants

The following monomeric STxB variants were synthetized and tested:
STxB-T1-A-N₃: STxB with SEQ ID NO: 2, comprising a substitution of Thr 1 with 3-azido-L-alanine;
STxB-D3-K-N₃: STxB with SEQ ID NO: 2, comprising a substitution of Asp 3 with 6-azido-L-lysine;
STxB-T6-K-N₃: STxB with SEQ ID NO: 2, comprising a substitution of Thr 6 with 6-azido-L-lysine;
STxB-K8-K-N₃: STxB with SEQ ID NO: 2, comprising a substitution of Lys 8 with 6-azido-L-lysine;
STxB-E10-K-N₃: STxB with SEQ ID NO: 2, comprising a substitution of Glu 10 with 6-azido-L-lysine;
STxB-Y11-K-N₃: STxB with SEQ ID NO: 2, comprising a substitution of Tyr 11 with 6-azido-L-lysine;
STxB-Y11-F4-F—CH₂—N₃: STxB with SEQ ID NO: 2, comprising a substitution of Tyr 11 with 4-azidomethyl-L-phenylalanine;
STxB-K23-K-N₃: STxB with SEQ ID NO: 2, comprising a substitution of Lys 23 with 6-azido-L-lysine;
STxB-D26-K-N₃: STxB with SEQ ID NO: 2, comprising a substitution of Asp 26 with 6-azido-L-lysine;
STxB-K27-K-N₃: STxB with SEQ ID NO: 2, comprising a substitution of Lys 27 with 6-azido-L-lysine;
STxB-T49-K-N₃: STxB with SEQ ID NO: 2, comprising a substitution of Thr 49 with 6-azido-L-lysine;
STxB-K53-K-N₃: STxB with SEQ ID NO: 2, comprising a substitution of Lys 53 with 6-azido-L-lysine;
STxB-H58-A-N₃: STxB with SEQ ID NO: 2, comprising a substitution of His 58 with 3-azido-L-alanine;
STxB-N59-K-N₃: STxB with SEQ ID NO: 2, comprising a substitution of Asn 59 with 6-azido-L-lysine;
STxB-R69-K-N₃—CONH₂: STxB with SEQ ID NO: 2, comprising a substitution of Arg 69 with 6-azido-L-lysine;
STxB-C-ter-70-A-N₃—CONH₂: STxB with SEQ ID NO: 2, comprising an addition in C-terminal (after Arg 69) of a 3-azido-L-alanine (SEQ ID NO: 23);
STxB-C-ter-70-K-N₃—CONH₂: STxB with SEQ ID NO: 2, comprising an addition in C-terminal (after Arg 69) of a 6-azido-L-lysine;
STxB-M48-Nle: STxB with SEQ ID NO: 2, comprising a substitution of Met 48 with L-norleucine.

Reagents

Solid-phase synthesis of full length STxB variants was performed on a Prelude Instrument (Gyros protein Technologies), at 12.5 μmol scale, using a Fmoc-Arg(Pbf)-Wang low loading resin (Novabiochem), except for STxB-C-ter-70-A-N₃—CONH₂ and STxB-C-ter-70-K-N₃—CONH₂ variants. The Fmoc-Arg(Pbf)-Wang low loading resin is pre-loaded with an arginine residue comprising an α amino-protecting group (fluorenylmethyloxycarbonyl; Fmoc), and a side-chain protecting-group (2,2,4,6,7-pentamethyldihydrobenzofuran-5-sulfonyl; Pbf). For STxB-C-ter-70-A-N₃—CONH₂ and STxB-C-ter-70-K-N₃—CONH₂ variants, a H-Rink amide ChemMatrix® resin (Sigma-Aldrich) was used.

Amino acids and pseudoprolines were purchased from Novabiochem.

N-methylmorpholine (NMM), acetic anhydride (Ac₂O), acetic acid, thioanisole, anisole, triisopropylsilane (TIS), sodium phosphate monobasic, sodium phosphate dibasic and dimethyl sulfoxide (DMSO) were obtained from Sigma Aldrich.

2-(6-chloro-1H-benzotriazol-1-yl)-N,N,N′,N′-tetramethylaminium hexafluorophosphate (HCTU) was obtained from VWR.

Dichloromethane (DCM), piperidine and diethyl ether were purchased from Carlo Erba.

Dimethylformamide (DMF) was obtained from Merck Millipore.

N-methyl-2-pyrrolidone (NMP) was obtained from BDH Chemicals.

Trifluoroacetic acid (TFA) was purchased from Fisher Scientific.

Guanidine hydrochloride (GuHCl) was purchased from Calbiochem.

STxB Variants Synthesis

The resin was swelled twice in 3 mL DCM for 30 seconds with mixing, then once in 3 mL NMP for 5 minutes with mixing.

Standard Synthesis Cycle

The synthesis workflow was set as follows on the Prelude Instrument, with one cycle being defined as substeps (1) to (6) defined below, each cycle leading to the addition of one amino acid to the growing peptide, in a linear C- to N-terminal direction following SEQ ID NO: 2 (except defined mutation for each variant).

(1) Deprotection
This substep was carried out twice in a row per cycle, with 2 mL of 20% piperidine in NMP for 3 minutes each time, with mixing.

(2) Washes
This substep was carried out three times in a row per cycle, with 3 mL of NMP for seconds each time, with mixing.

(3) Coupling
This substep was carried out once 15 minutes or twice 5 minutes per cycle, with 1300 μL of Fmoc-protected amino acid (200 mM in NMP=20.8 eq.; except for cysteine residues: 200 mM in DMF=20.8 eq), 1000 μL of HCTU (250 mM in NMP=20 eq.) and 500 μL of NMM (1 M in NMP=40 eq.), with mixing.

(4) Washes
This substep was carried out twice in a row per cycle, with 3 mL of NMP for seconds each time, with mixing.

(5) Capping
This substep was carried out once per cycle, with 2000 μL of Ac₂O (250 mM in NMP) and 500 μL of NMM (1 M in NMP=40 eq.) for 5 minutes, with mixing.

(6) Washes
This substep was carried out three times in a row per cycle, with 3 mL of NMP for seconds each time, with mixing.

Dipeptides Val 5-Thr 6, Asp 18-Thr 19, Phe 30-Thr 31, Leu 41-Ser 42, Val 50-Thr 51, and Phe 63-Ser 64 with respect to SEQ ID NO: 2 numbering were coupled in step 3) in a pseudoproline dipeptide form.

For some positions, coupling (step 3) was either repeated more times and/or carried out for a longer duration. These positions include: Cys 4, Thr 12, Thr 21, Asn 35, Leu 36, Leu 39, Ile 45, Thr 49, Cys 57, Thr 53, Val 65 with respect to SEQ ID NO: 2 numbering, in addition to pseudoproline positions and to the position of the unnatural amino acid incorporation.

Final Deprotection

Once the whole monomeric STxB variants were synthetized, the final α amino-protecting group (i.e., the Fmoc protecting group borne by the threonine residue in position 1 of SEQ ID NO: 2), were removed, according to the following substeps:

(1) Deprotection
This substep was carried out twice in a row, with 2 mL of 20% piperidine in NMP for 3 minutes each time, with mixing.

(2) NMP Wash
This substep was carried out once, with 3 mL of NMP for 30 seconds, with mixing.

(3) DCM Washes
This substep was carried out four times in a row, with 3 mL of DCM for 30 seconds each time, with mixing.

Cleavage

The monomeric STxB variants were cleaved from the resin in 5 mL TFA:thioanisole:anisole:TIS:H₂O (82.5:5:5:2.5:5) for 2 hours under stirring. Under these conditions, side-chain protecting groups optionally borne by the amino acid residues (in particular non-aliphatic amino acid residues) were also removed.

The cleavage solution was then precipitated in 40 mL cold diethyl ether.

After 3 washes with 45 mL cold diethyl ether, the precipitate was air dried. The precipitate was then mixed in 15 mL 10% acetic acid in water/acetonitrile mix and lyophilized

Oxidation and Folding

Purified lyophilized monomeric STxB variants were dissolved to 0.5 mg/mL in oxidation buffer (7 M GndHCl, 50 mM sodium phosphate, 2% DMSO, pH adjusted to 8).

The solution was then incubated under stirring at 37° C. for 24 hours, to form a disulfide bond between Cys 4 and Cys 57 of SEQ ID NO: 2.

The solution was then dialyzed at 4° C. with Slide-A-Lyzer™ G2 Dialysis Cassettes, 3.5 kDa MWCO from Thermo Scientific against the following:
3 M GndHCl, 50 mM sodium phosphate pH 8.0, 5 mM EDTA, for 6 to 10 hours;
1 M GndHCl, 50 mM sodium phosphate pH 8.0, 1 mM EDTA, overnight; PBS for 4 hours;
PBS for 4 hours; and
PBS overnight.

After removal from the dialysis cassette, the solution was centrifuged to remove the precipitate. Supernatant was kept and concentrated using centrifugal filters (Amicon Ultra Centrifugal filters, 10 kDa MWCO).

Concentration was measured from absorbance at 280 nm with a Nanodrop 2000, using the approximated extinction coefficient calculated from Gill and von Hippel coefficients with the following formula (Gill & von Hippel, 1989. Anal Biochem. 182(2):319-26):

ε_{280 nm}=5690×(number of Trp)+1280×(number of Tyr)+120×(number of Cystine)

This formula leads to ε_{280 nm}=8370 M⁻¹·cm⁻¹ for most STxB variants described, except for variants with Tyr 11 substitutions (STxB-Y11-K-N₃ and STxB-Y11-F4-F—CH₂—N₃), for which ε_{280 nm}=7090 M⁻¹·cm⁻¹.

Small aliquots were flash-frozen and stored at −20° C.

Intracellular Trafficking Assay by Immunofluorescence

Intracellular trafficking assays were performed on HeLa cells, cultured at 37° C. under % CO₂ in Dulbecco's modified Eagle's medium (DMEM, Invitrogen), supplemented with 10% heat-inactivated fetal bovine serum (FBS), 0.01% penicillin-streptomycin, 4 mM glutamine and 5 mM pyruvate.

Cells were plated the day before on lamellae in 4-well plates, 60 000 cells/well.

Binding

Cells were incubated for 30 minutes at 4° C. in presence of 500 μL STxB dilution in cold complete medium (0.2 μM monomer concentration), then washed 3 times with 500 μL PBS with Ca²⁺ and Mg²⁺ (PBS⁺⁺).

Internalization

500 μL of complete medium preheated at 37° C. was added on cells. Cells were incubated for 50 minutes at 37° C., then washed 3 times with PBS⁺⁺.

Fixation

Cells were treated with 500 μL of 4% paraformaldehyde (PFA) during 20 minutes, then washed once with 50 mM of NH₄Cl, and incubated with 50 mM of NH₄Cl for at least minutes.

Permeabilization

Cells were washed 3 times with 500 μL of PBS/BSA/Saponin 1× (1×PBS/1.0% BSA/0.1% Saponin), and then incubated at room temperature for 30 minutes in presence of 500 μL of PBS/BSA/Saponin 1×.

Incubation with Antibodies

Lamellae were incubated with 30 μL of primary antibody dilution into PBS/BSA/Saponin 1× for 30 minutes at room temperature, then washed 3 times with PBS/BSA/Saponin 1×.

Primary antibodies used were the mouse monoclonal clone 13C4 anti-STxB antibody (Strockbine et al., 1985. Infect Immun. 50(3):695-700), at 1/250 dilution; and a home-made rabbit polyclonal antibody against the Golgi marker Giantin, used at 1/100 dilution.

Same was done with the secondary antibodies (anti-mouse Cy3 and anti-rabbit A488 used at 1/100 dilution each).

Slide Preparation

Lamellae were washed in water and then added on slides on 6 μL of Fluoromount-G®+Hoechst. Polymerization was allowed for 30 minutes at 37° C.

Cy3-NHS Labelling

Immunolabeling with the monoclonal α-STxB antibody 13C4 gave less or no signal in the intracellular trafficking assay for STxB-T1-A-N₃ and STxB-H58-A-N₃. In order to know whether this was due to lower binding of the 13C4 antibody to these STxB variants or whether this was due to decreased functionality of the variants themselves, these two variants along with a STxB WT control were directly conjugated with a fluorophore and tested with the intracellular trafficking assay (without 13C4 antibody labelling).

Samples were labelled with NHS-Cy3 from Amersham kit PA23001 at 21° C., 800 rpm for 2 hours, before quenching by addition of 100 mM Tris pH 7.4 (final concentration: 20 mM Tris). Excess NHS-Cy3 was removed with Zeba spin desalting columns 7 kDa MWCO (ThermoFisher Scientific).

Direct fluorophore labelling confirmed lower signal with STxB-T1-A-N₃ in the intracellular trafficking assay. On the contrary, STxB-H58-A-N₃ showed normal signal and colocalization to the Golgi.

Conjugation Test of Azide Variants with DBCO-NH₂

Conjugation tests with dibenzocyclooctyne-amine (DBCO-NH₂; CAS 1255942-06-3, from Iris biotech) were performed by addition of 5 μL 5.4 mM DBCO-NH₂ solution in DMSO to 45 μL of 200 μM (monomer concentration) STxB variant solution in PBS, corresponding to 3 eq. DBCO-NH₂ compared to STxB monomers.

The reaction was left overnight at 21° C. under stirring at 750 rpm.

UPLC-MS analysis was done to confirm conjugated product formation.

UPLC-MS Analyses

Samples were analyzed with a Waters UPLC-MS comprising an ACQUITY UPLC H-Class sample manager, an ACQUITY UPLC PDA eLambda Detector, and a Single Quadrupole Detector 2 for electron spray ionization mass spectra. An ACQUITY UPLC BEH C18 1.7 μm 2.1×50 mm column was used.

Solvents were:
A: 0.1% formic acid in Milli-Q water;
B: 0.1% formic acid in acetonitrile.

Cycles were:
0.2 minutes 5% B for accumulation at the head of the column;
2.3 minutes linear gradient from 5% to 95% B;
minutes 100% B for washing; and
1 minute at 5% B for column equilibration.

Results

Results are given in Table 1.

In conclusion, the data presented above show that several functional STxB variant pentamers and conjugates thereof could be obtained via solid-phase synthesis.

As is however summarized in Table 1, intracellular trafficking assay for the STxB-T1-A-N₃ was negative, despite good synthesis, oxidation and folding yields. As for variant STxB-D26-K-N₃, synthesis yield was very high, but oxidation and folding yields were the lowest obtained with all variants. Despite a correct intracellular trafficking, conjugation with DBCO-NH₂ mainly resulted in precipitation.

Finally, variant STxB-T6-K-N3 also showed very low oxidation and folding yields, which render the production of this variant difficult if not impossible to scale up.

It is worth noting that the position of the amino acid substitution on STxB is not a cause for these issues.

Table 1

Unless indicated otherwise (e.g., —CONH₂), all STxB variants were terminated by a free carboxyl group (—COOH).

Position on STxB: “low” means located at or near the protein-membrane interface; “high” means located opposite from the protein-membrane interface; “medium” means located in-between the protein-membrane interface and the opposite side. Intracellular trafficking assay: “Y” means that the assay was positive; “N” means that the assay as negative. Conjugation with DBCO-NH₂: “Y” means that the conjugation was successful; “N” means that the conjugation was not successful.

| | | | | Synthesis | | | | |
|---|---|---|---|---|---|---|---|---|
| | | | Crude | yield (from | | | | |
| | | Synthesis | peptide | crude | Oxidation | Oxidation | | |
| | | yield (from | mass | peptide | and folding | and folding | Intracellular | Conjugation |
| | Position on | Fmoc | obtained | mass | yield (%) | yield (%) | trafficking | with DBCO- |
| STxB variants | STxB | deprotection) | (mg) | obtained) | (1^{st} round) | (2^{nd} round) | assay | NH₂) |
| STxB-T1-A-N₃ | Low | 69% | 54.5 | 57% | 34 | 36 | N | Y |
| STxB-D3-K-N₃ | Low | 69% | 43.8 | 45% | 29 | 32 | Y | Y |
| STxB-T6-K-N₃ | High | 42% | 63.7 | 66% | 14 | 17 | Y | Y |
| STxB-K8-K-N₃ | High | 56% | 77.3 | 80% | 38.1 | 33 | Y | Y |
| STxB-E10-K-N₃ | High | 70% | 72.9 | 76% | 26 | 34 | Y | Y |
| STxB-Y11-K-N₃ | Medium/high | 58% | 73.1 | 76% | 24 | 34 | Y | Y |
| STxB-Y11-F4- | Medium/high | 43% | 62.5 | 65% | 27 | 28 | Y | Y |
| F-CH₂-N₃ | | | | | | | | |
| STxB-K23-K-N₃ | Medium/high | ? | 55 | 57% | 37 | 38 | Y | Y |
| STxB-D26-K-N₃ | High | 59% | 81.1 | 84% | 9 | 15 | Y | N |
| STxB-K27-K-N₃ | Medium | 57% | 64.4 | 67% | 34.5 | 31 | Y | Y |
| STxB-T49-K-N₃ | High | 51% | 92.5 | 96% | 28 | 32 | Y | Y |
| STxB-K53-K-N₃ | Low | 50% | 71.7 | 74% | 27 | 24 | Y | Y |
| STxB-H58-A-N₃ | Low | 44% | 64 | 67% | 33 | 25 | Y | Y |
| STxB-N59-K-N₃ | Low | 45% | 68.5 | 71% | 29 | 24 | Y | Y |
| STxB-R69-K- | High | 45% | 61.1 | 64% | 22 | 22 | Y | Y |
| N₃-CONH₂ | | | | | | | | |
| STxB-C-ter-70- | High | / | 18.6 | 19% | 29 | / | Y | Y |
| A-N₃-CONH₂ | | | | | | | | |
| STxB-C-ter-70- | High | 61% | 25.3 | 26% | 21 | / | Y | Y |
| K-N₃-CONH₂ | | | | | | | | |
| STxB-M48-Nle | High | 59% | 77.2 | 80.5% | 26 | 30 | Y | / |

Conclusion

Together, these data indicate that some positions on the STxB protein are more prone to amino acid substitution/addition and conjugation than others, paving the way to the production of STxB conjugates of pharmaceutical grade purity on a large scale: Asp 3, Lys 8, Glu 10, Tyr 11, Lys 23, Lys 27, Thr 49, Lys 53, His 58, Asn 59, Arg 69 and the C-terminal extremity (after Arg 69) (SEQ ID NO: 2 numbering).

Different methods for the incorporation of modified amino acid residues into proteins exist. One of such methods in recombinant production is termed “stop codon suppression”: the amber stop codon (UAG) in a host cell is reassigned to the modified amino acid residue of choice (Xie & Schultz, 2005. Methods. 36(3):227-38), with a recombinant aminoacyl-tRNA synthetase/amber suppressor tRNA pair provoking the site-specific incorporation of the modified amino acid residue in response to the amber stop codon (Dumas et al., 2015. Chem Sci. 6(1):50-69). However, this method has shown its limits in that it yields highly contaminated samples comprising a majority (z 60%) of wild-type species.

Using solid-phase synthesis, a tight monitoring at each incorporation round can be carried out, resulting in highly homogenous samples comprising a large majority—if not the totality—of the desired variant species. Finally, the use of solid-phase synthesis avoids other classical drawbacks encountered with recombinant production, such as endotoxin contamination to name but one.

### Example 2

We aimed at evaluating the capacity of the STxB variants to be conjugated with an antigenic peptide and to elicit an immune response upon vaccination.

This proof of concept was carried out using an N-terminally extended version of the OVA₂₅₇₋₂₆₄ peptide (also known as SL8 peptide, with SEQ ID NO: 21).

Materials and Methods

JU57: Recombinant STxB-Cys/Bromoacetyl-SL8 Conjugate

A recombinant STxB pentamer (rSTxB) comprising an additional cysteine residue at the C-terminal extremity of SEQ ID NO: 2 (SEQ ID NO: 22) was produced as previously described in Mallard & Johannes (2003. Methods Mol Med. 73:209-220). This rSTxB was dialyzed against 50 mM borate buffer, 150 mM NaCl pH 9.

SEQ ID NO: 22
TPDCVTGKVEYTKYNDDDTFTVKVGDKELFTNRWNLQSLLLSAQITGMT
VTIKTNACHNGGGFSEVIFRC

A conjugation reaction was carried out at 21° C., 750 rpm overnight with 3 molar equivalents of bromoacetyl SL8 peptide (Br—CH₂CO-QLESIINFEKL; SEQ ID NO: 21) and 10% DMSO to solubilize the peptide.

The conjugate, named “JU57” in the following, was purified from excess-free SL8 peptide by several dialysis steps at 4° C. against PBS, using Slide-A-Lyzer 20K MWCO dialysis cassettes (Thermo Scientific).

Conjugate formation and absence of remaining free SL8 peptide after purification were validated by UPLC-MS.

ABILC2: Synthetic STxB-C-Ter-70-A-N₃—CONH₂/DBCO-PEG₄-SL8 Conjugate

DBCO-PEG4-SL8 Synthesis

Cys-SL8 Peptide Synthesis

A Cys-SL8 peptide (CQLESIINFEKL-OH; SEQ ID NO: 23) was synthesized on a Prelude instrument at a 25 μmol scale.

Deprotection was carried out with 2 mL of 20% piperidine in NMP for 2 minutes (repeated twice), followed by 3 mL of NMP washes for 30 seconds (repeated 3 times).

Coupling steps were carried out at least twice 10 minutes with 1.3 mL of 200 mM amino acid in NMP, 1 mL 250 mM HCTU in NMP and 0.5 mL 1 M NMM in NMP, followed by 3 mL of NMP washes for 30 seconds (repeated twice).

Capping was carried out for 5 minutes with 2 mL of 250 mM acetic anhydride in NMP and 0.5 mL of 1 M NMM in NMP, followed by 3 mL of NMP washes for 30 seconds (repeated 3 times).

Cleavage was carried out with 5 mL TFA/thioanisole/anisole/TIS/H₂O (82.5/5/5/2.5/5) for 2 hours under stirring. The cleavage solution was then precipitated in 40 mL cold diethyl ether. After 3 washes with cold diethyl ether, the precipitate was air-dried and dissolved in 10% acetic acid and lyophilized.

DBCO-PEG4/SL8 Coupling

mg of crude Cys-SL8 peptide were reacted with 1.1 molar equivalent of commercial dibenzocyclooctyne (DBCO)-PEG4-maleimide (Iris Botech; CAS: 1480516-75-3; MW=674.74 g/mol) in 4 mL of 40% acetonitrile/60% of 50 mM ammonium bicarbonate buffer pH 7 for 1 hour, prior to freeze-drying.

The product was purified by HPLC with a Water Xbridge BEH 300 Prep C18 5 μm mm column, a Waters 2545 Quaternary Gradient Module, a Waters 2998 Photodiode Array Detector, and a Waters FlexInject. A 30-minute 30 mL/min gradient from 5 to 100% of acetonitrile in water was used (without formic acid), yielding after freeze-drying 2.5 mg of DBCO-PEG₄-SL8 in powder form (yield=17%; purity=78%; MS m/z C₁₀₀H₁₄₇N₁₉O₂₉S [M⁺H⁺]⁺ calculated=2112.4, found=2112.4; [M⁺2H⁺]²⁺ calculated=1056.7, found=1056.6; [M⁺3H⁺]³⁺ calculated=704.8, found=704.8; [M⁻H⁺]⁻ calculated=2110.4, found=2110.9).

Conjugation Between Synthetic STxB-Cter-70-A-N3-CONH2 and DBCO-PEG4-SL8 (ABILC2)

Synthetic STxB-Cter-70-A-N₃—CONH₂ (SEQ ID NO: 24) was synthetized as described in Example 1, and further dialyzed for 4 hours against 50 mM borate buffer, 150 mM NaCl pH 9, using a Slide-A-Lyzer 0.5-3 mL 10K MWCO dialysis cassette (Thermo Scientific).

A conjugation reaction was carried out at 21° C., 750 rpm overnight with 2 molar equivalents of DBCO-PEG₄-SL8 and 10% DMSO to solubilize the peptide.

The excess of free SL8 peptide was removed using Zeba™ Spin Desalting columns (7K MWCO, 2 mL; Thermo Scientific), followed by a dialysis step against PBS overnight using Slide-A-Lyzer 0.5-3 mL 10K MWCO dialysis cassettes (Thermo Scientific).

The conjugate, named “ABILC2” in the following, was purified from excess-free SL8 peptide using Zeba™ Spin Desalting columns (7K MWCO, 2 mL; Thermo Scientific), followed by a dialysis step against PBS overnight, using Slide-A-Lyzer 0.5-3 mL 10K MWCO dialysis cassettes (Thermo Scientific).

Conjugate formation and absence of remaining free SL8 peptide after purification were validated by UPLC-MS (data not shown).

Validation of Conjugate Functionality in Cells

The functionality of JU57 and ABILC2 was assessed on cells by immunofluorescence with the intracellular trafficking assay, as described in Example 1.

Slides were visualized on an upright Leica DM6B microscope with a sCMOS Orca Flash 4.0 V2 camera (pixel size: 6.5 μm) from Hamamatsu with illumination by Metal-Halide EL 6000 lamp from Leica and a 100×HCX PL APO objective. Fiji ImageJ software (National Institutes of Health; Schindelin et al., 2012. Nat Methods. 9(7):676-682) was used for image processing.

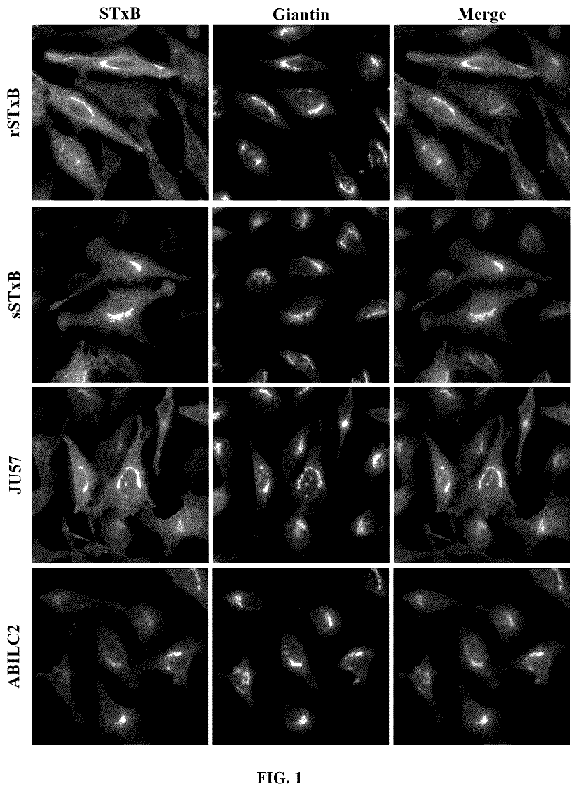

Both conjugates colocalized with the Golgi after 50 minutes of incubation at 37° C., similarly to the unconjugated rSTxB-Cys and synthetic STxB-Cter-70-A-N₃—CONH₂, validating the functionality of the conjugates on cells (FIG. 1).

Results

C57BL/6 mice were immunized at day 0 and day 14 via the intranasal route with 20 μg of JU57, ABILC2 or free SL8 peptide. The vaccines were combined with cyclic diguanylate (c-di-GMP) as adjuvant. The mice were then sacrificed at day 21.

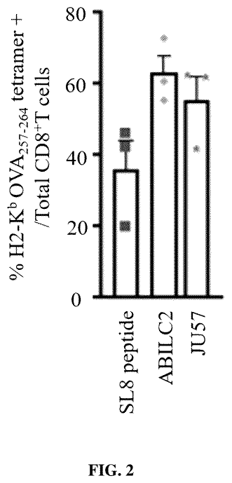

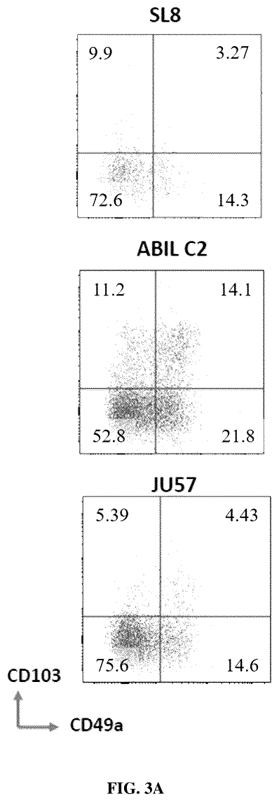

We were able to show that the synthetic STxB-C-ter-70-A-N3-CONH₂/DBCO-PEG₄-SL8 conjugate, namely ABILC2, was far more efficient than the free ovalbumin-derived SL8 peptide to elicit specific anti-OVA CD8⁺ T cells (FIG. 2). ABILC2 was also slightly more efficient than the conventional recombinant STxB-Cys/bromoacetyl-SL8 conjugate, namely JU-57, in terms of ability to induce specific anti-OVA CD8⁺ T cells (FIG. 2).

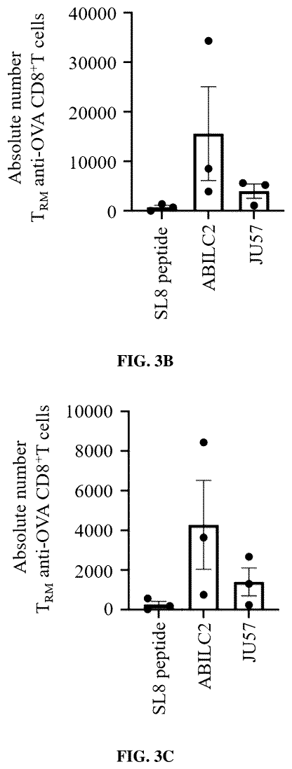

Most surprisingly, ABILC2 was able to elicit resident memory CD8⁺ T cells (TRM) defined by the expression of CD103 and CD49a (Mami-Chouaib et al., 2018. J Immunother Cancer. 6(1):87) at substantially higher levels than JU57 both in the lung parenchyma (FIGS. 3A & C) and in broncho-alveolar lavages (BAL) (FIG. 3B).

The teams of L. Johannes and E. Tartour have shown that anti-tumor T_{RM} are the main effectors mediating the efficacy of cancer vaccine especially for mucosal tumors (Nizard et al., 2017. Nat Commun. 8:15221; Karaki et al., 2021. J Immunother Cancer. 9(3):e001948). These cells are also required for the complete neutralization of mucosal infection (Hassan et al., 2020. Cell. 183(1): 169-184.e13; Perdomo et al., 2016. mBio. 7(6):e01686-16).

Conclusion

Altogether, these data demonstrate the superiority of synthetic STxB variants to act as vectors capable of delivering specific antigens into antigen-presenting cells, and to trigger efficient immune responses.

1-17. (canceled)

18. A composition comprising at least 50% of isolated, modified monomer of a Shiga toxin B-subunit (STxB) protein or of a variant thereof out of the total monomers of the STxB protein or of the variant thereof in the composition,
wherein said modified monomer of the STxB protein or of the variant thereof comprises at least one of:
an addition of a reactive unnatural amino acid residue at the C-terminal extremity, and/or
a substitution with a reactive unnatural amino acid residue at one or several amino acid positions selected from the group consisting of Asp 3, Lys 8, Glu 10, Tyr 11, Lys 23, Lys 27, Thr 49, Lys 53, His 58, Asn 59, and Arg 69,
reference made to SEQ ID NO: 2 numbering, or at an equivalent position in a variant thereof.

19. The composition according to claim 18, wherein said monomer of the STxB protein or of the variant thereof does not comprise a substitution with a reactive unnatural amino acid residue at amino acid positions Thr 1, Thr 6 and Asp 26, according to SEQ ID NO: 2 numbering, or at equivalent positions in a variant thereof.

20. The composition according to claim 18, wherein said reactive unnatural amino acid residue comprises a functional group selected from the group consisting of azide, alkyne, aldehyde, keto, beta-diketo, alkoxyamine, acyl hydrazide, dehydroalanine, thioester, ester, boronate, halide, acetylenic, olefinic, vicinal thiol amine, and norbornene moieties.

21. The composition according to claim 18, wherein said reactive unnatural amino acid residue is selected from the group consisting of 6-azido-L-lysine, 3-azido-L-alanine and 4-azidomethyl-L-phenylalanine.

22. The composition according to claim 18, wherein said monomer of the STxB protein or of the variant thereof is selected from the group consisting of:
a STxB protein comprising an addition at its C-terminal extremity of a 3-azido-L-alanine,
a STxB protein comprising an addition at its C-terminal extremity of a 6-azido-L-lysine;
a STxB protein comprising a substitution of Asp 3 into 6-azido-L-lysine,
a STxB protein comprising a substitution of Lys 8 into 6-azido-L-lysine,
a STxB protein comprising a substitution of Glu 10 into 6-azido-L-lysine,
a STxB protein comprising a substitution of Tyr 11 into 6-azido-L-lysine,
a STxB protein comprising a substitution of Tyr 11 into 4-azidomethyl-L-phenylalanine,
a STxB protein comprising a substitution of Lys 23 into 6-azido-L-lysine,
a STxB protein comprising a substitution of Lys 27 into 6-azido-L-lysine,
a STxB protein comprising a substitution of Thr 49 into 6-azido-L-lysine,
a STxB protein comprising a substitution of Lys 53 into 6-azido-L-lysine,
a STxB protein comprising a substitution of His 58 into 3-azido-L-alanine,
a STxB protein comprising a substitution of Asn 59 into 6-azido-L-lysine, and
a STxB protein comprising a substitution of Arg 69 into 6-azido-L-lysine;
reference made to SEQ ID NO: 2 numbering, or at an equivalent position in a variant thereof.

23. The composition according to claim 18, wherein said monomer of the STxB protein or of the variant thereof has an amino acid sequence with at least 75% global sequence identity to an amino acid sequence selected from the group comprising SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 and SEQ ID NO: 20.

24. The composition according to claim 18, wherein said monomer of the STxB protein or of the variant thereof further comprises a substitution of Met 48 with L-norleucine, reference made to SEQ ID NO: 2 numbering, or at an equivalent position in a variant thereof.

25. The composition according to claim 18, wherein said monomer of the STxB protein or of the variant thereof is not a recombinant protein.

26. The composition according to claim 18, wherein said modified monomer of the STxB protein or of the variant thereof is a conjugate, comprising a payload bound thereto through the reactive unnatural amino acid residue, optionally via a linker.

27. The composition according to claim 26, wherein the payload is selected from the group consisting of chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, antigens, hormones, coding or non-coding oligonucleotides, photodetectable labels, contrast agents, and radiolabels.

28. A composition comprising at least 50% of modified pentamers of a Shiga toxin B-subunit (STxB) protein or of a variant thereof out of the total pentamers of the STxB protein or of the variant thereof in the composition,
wherein said modified pentamers of the STxB protein or of the variant thereof comprise at least one modified monomer as defined in claim 18.

29. The composition according to claim 28, wherein said modified pentamers are conjugates comprising at least one STxB monomer conjugate, said conjugate comprising a payload bound thereto through the reactive unnatural amino acid residue, optionally via a linker.

30. The composition according to claim 28, wherein said modified pentamers of the STxB protein or of the variant thereof comprise five of said at least one modified monomers.

31. The composition according to claim 28, wherein said modified pentamers of the STxB protein or of the variant thereof retain their ability to bind to the glycosphingolipid Gb3/CD77.

32. A method for treating a disease in a subject in need thereof or for vaccinating a subject, comprising administrating a composition according to claim 17 to the subject, wherein the disease is selected from cancer, infectious diseases, immune disorders and inflammatory disorders.

33. A method for treating a disease in a subject in need thereof or for vaccinating a subject, comprising administrating a composition according to claim 28 to the subject, wherein the disease is selected from cancer, infectious diseases, immune disorders and inflammatory disorders.

34. A method of medical imaging of a subject in need thereof, wherein a composition according to claim 18 is administered to the subject as a contrast agent and wherein:
the STxB monomer conjugate comprises a payload selected from the group consisting of photodetectable labels, contrast agents and radiolabels.

35. A method of medical imaging of a subject in need thereof, wherein a composition according to claim 28 is administered to the subject as a contrast agent and wherein:
the STxB pentamer conjugate comprises a payload selected from the group consisting of photodetectable labels, contrast agents and radiolabels.

36. The method of medical imaging according to claim 34, wherein said method is an in vivo method of diagnosing a disease in a subject in need thereof, wherein said composition is administered to the subject as a contrast agent and wherein:
the STxB monomer conjugate comprises a payload selected from the group consisting of photodetectable labels, contrast agents and radiolabels.

37. The method of medical imaging according to claim 35, wherein said method is an in vivo method of diagnosing a disease in a subject in need thereof, wherein said composition is administered to the subject as a contrast agent and wherein:
the STxB pentamer conjugate comprises a payload selected from the group consisting of photodetectable labels, contrast agents and radiolabels.


SEQUENCE LISTING (24 sequences)

SEQ ID NO: 1 - protein, 89 aa, Shigella dysenteriae
  STxB from Shigella dysenteriae (Q7BQ98-1)
MKKTLLIAASLSFFSASALATPDCVTGKVEYTKYNDDDTFTVKVGDKELFTNRWNLQSLLLSAQITGMTVTIKTNACHNGGGFSEVIFR

SEQ ID NO: 2 - protein, 69 aa, Artificial Sequence
  STxB from Shigella dysenteriae (Q7BQ98-1) devoid of its signal peptide
TPDCVTGKVEYTKYNDDDTFTVKVGDKELFTNRWNLQSLLLSAQITGMTVTIKTNACHNGGGFSEVIFR

SEQ ID NO: 3 - protein, 89 aa, Escherichia coli
  STx1cB from Escherichia coli (Q47641-1)
MKKILLIAASLSFFSASVLAAPDCVTGKVEYTKYNDDDTFTVKVGDKELFTNRWNLQSLLLSAQITGMTVTIKTNACHNGGGFSEVIFR

SEQ ID NO: 4 - protein, 69 aa, Artificial Sequence
  STx1cB from Escherichia coli (Q47641-1) devoid of its signal peptide
APDCVTGKVEYTKYNDDDTFTVKVGDKELFTNRWNLQSLLLSAQITGMTVTIKTNACHNGGGFSEVIFR

SEQ ID NO: 5 - protein, 89 aa, Escherichia coli
  STx1dB from Escherichia coli (Q83XK2-1)
MKKVLLIAVSLSFLSASVLAAPDCVTGKVEYTKYNDDDTFTVKVADKELFTNRWNLQSLLLSAQITGMTVTIKTTACHNGGGFSEVIFR

SEQ ID NO: 6 - protein, 69 aa, Artificial Sequence
  STx1dB from Escherichia coli (Q83XK2-1) devoid of its signal peptide
APDCVTGKVEYTKYNDDDTFTVKVADKELFTNRWNLQSLLLSAQITGMTVTIKTTACHNGGGFSEVIFR

SEQ ID NO: 7 - protein, 89 aa, Escherichia coli
  STx2B from Escherichia coli (Q8X531-1)
MKKMFMAVLFALASVNAMAADCAKGKIEFSKYNEDDTFTVKVDGKEYWTSRWNLQPLLQSAQLTGMTVTIKSSTCESGSGFAEVQFNND

SEQ ID NO: 8 - protein, 70 aa, Artificial Sequence
  STx2B from Escherichia coli (Q8X531-1) devoid of its signal peptide
ADCAKGKIEFSKYNEDDTFTVKVDGKEYWTSRWNLQPLLQSAQLTGMTVTIKSSTCESGSGFAEVQFNND

SEQ ID NO: 9 - protein, 89 aa, Escherichia coli
  STx2cB from Escherichia coli (Q07871-1)
MKKMFMAVLFALVSVNAMAADCAKGKIEFSKYNENDTFTVKVAGKEYWTSRWNLQPLLQSAQLTGMTVTIKSSTCESGSGFAEVQFNND

SEQ ID NO: 10 - protein, 70 aa, Artificial Sequence
  STx2cB from Escherichia coli (Q07871-1) devoid of its signal peptide
ADCAKGKIEFSKYNENDTFTVKVAGKEYWTSRWNLQPLLQSAQLTGMTVTIKSSTCESGSGFAEVQFNND

SEQ ID NO: 11 - protein, 89 aa, Escherichia coli
  STx2dB from Escherichia coli (Q8GGL0-1)
MKKMFMAVLFALVSVNAMAADCAKGKIEFSKYNENDTFTVKVDGKEYWTSRWNLQPLLQSAQLTGMTVTIKSSTCASGSGFAEVQFNND

SEQ ID NO: 12 - protein, 70 aa, Artificial Sequence
  STx2dB from Escherichia coli (Q8GGL0-1) devoid of its signal peptide
ADCAKGKIEFSKYNENDTFTVKVDGKEYWTSRWNLQPLLQSAQLTGMTVTIKSSTCASGSGFAEVQFNND

SEQ ID NO: 13 - protein, 87 aa, Escherichia coli
  STx2eB from Escherichia coli (Q47644-1)
MKKMFIAVLFALVSVNAMAADCAKGKIEFSKYNEDNTFTVKVSGREYWTNRWNLQPLLQSAQLTGMTVTIISNTCSSGSGFAQVKFN

SEQ ID NO: 14 - protein, 68 aa, Artificial Sequence
  STx2eB from Escherichia coli (Q47644-1) devoid of its signal peptide
ADCAKGKIEFSKYNEDNTFTVKVSGREYWTNRWNLQPLLQSAQLTGMTVTIISNTCSSGSGFAQVKFN

SEQ ID NO: 15 - protein, 87 aa, Escherichia coli
  STx2fB from Escherichia coli (Q47646-1)
MKKMIIAVLFGLFSANSMAADCAVGKIEFSKYNEDDTFTVKVSGREYWTNRWNLQPLLQSAQLTGMTVTIISNTCSSGSGFAQVKFN

SEQ ID NO: 16 - protein, 68 aa, Artificial Sequence
  STx2fB from Escherichia coli (Q47646-1) devoid of its signal peptide
ADCAVGKIEFSKYNEDDTFTVKVSGREYWTNRWNLQPLLQSAQLTGMTVTIISNTCSSGSGFAQVKFN

SEQ ID NO: 17 - protein, 87 aa, Escherichia albertii
  STx2fB from Escherichia albertii (C6L1N1-1)
MKKMIIAVLFGLFSANSMAADCAVGKIEFSKYNEDNTFTVRVSGREYWTNRWNLQPLLQSAQLTGMTVTIISNTCSSGSGFAQVKFN

SEQ ID NO: 18 - protein, 68 aa, Artificial Sequence
  STx2fB from Escherichia albertii (C6L1N1-1) devoid of its signal peptide
ADCAVGKIEFSKYNEDNTFTVRVSGREYWTNRWNLQPLLQSAQLTGMTVTIISNTCSSGSGFAQVKFN

SEQ ID NO: 19 - protein, 89 aa, Escherichia coli
  STx2gB from Escherichia coli (Q8VLE0-1)
MKKMFMAVLFALVSVNAMAADCAKGKIEFSKYNGDNTFTVKVDGKEYWTNRWNLQPLLQSAQLTGMTVTIKSNTCESGSGFAEVQFNND

SEQ ID NO: 20 - protein, 70 aa, Artificial Sequence
  STx2gB from Escherichia coli (Q8VLE0-1) devoid of its signal peptide
ADCAKGKIEFSKYNGDNTFTVKVDGKEYWTNRWNLQPLLQSAQLTGMTVTIKSNTCESGSGFAEVQFNND

SEQ ID NO: 21 - protein, 11 aa, Artificial Sequence
  SL8 peptide
QLESIINFEKL

SEQ ID NO: 22 - protein, 70 aa, Artificial Sequence
  STxB-Cys
TPDCVTGKVEYTKYNDDDTFTVKVGDKELFTNRWNLQSLLLSAQITGMTVTIKTNACHNGGGFSEVIFRC

SEQ ID NO: 23 - protein, 12 aa, Artificial Sequence
  Cys-SL8
CQLESIINFEKL

SEQ ID NO: 24 - protein, 70 aa, Artificial Sequence
  STxB-Cter-70-A-N3-CONH2
  MOD_RES 70: Ala is 3-azido-L-alanine
TPDCVTGKVEYTKYNDDDTFTVKVGDKELFTNRWNLQSLLLSAQITGMTVTIKTNACHNGGGFSEVIFRA